(19) United States
(12) Patent Application Publication
(10) Pub. No.: US 2010/0183504 A1
(43) Pub. Date: Jul. 22, 2010

(54) MULTIMODAL IMAGING PROBES FOR IN VIVO TARGETED AND NON-TARGETED IMAGING AND THERAPEUTICS

(76) Inventor: Fanqing Frank Chen, (US)

Correspondence Address: Weaver Austin Villeneuve & Sampson LLP, P.O. BOX 70250, OAKLAND, CA, 94612-0250, US

(21) Appl. No.: 12/663,225

(22) PCT Filed: Jun. 13, 2008
(86) PCT No.: PCT/US08/67009

§ 371 Date: Dec. 4, 2009

Related U.S. Application Data
(60) Provisional application No. 60/944,055, filed on Jun. 14, 2007.

(51) Int. Cl. A61K 51/12; A61K 49/18; C12N 5/071; A61P 35/00

(52) U.S. Cl. 424/1.29; 424/9.1; 435/325; 435/366; 424/9.3; 424/9.34

(57) ABSTRACT

In certain embodiments this invention provides a nanoparticle-based technology platform for multimodal in vivo imaging and therapy. The nanoparticle-based probes detects diseased cells by MRI, PET or deep tissue Near Infrared (NIR) imaging, and are capable of detecting diseased cells with greater sensitivity than is possible with existing technologies. The probes also target molecules that localize to normal or diseased cells, and initiates apoptosis of diseased cells.

### CROSS-REFERENCE TO RELATED APPLICATIONS

This application claims benefit of and priority to U.S. Ser. No. 60/944,055, filed on Jun. 14, 2007, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made during work supported by U.S. Department of Energy under Contract No. DE-ACO2-05CH11231 and Contract No. W-7405-Eng-48. The government has certain rights in this invention.

### FIELD OF THE INVENTION

This invention relates to using multimodal imaging probes for use in in vivo targeted and non-targeted imaging, specifically as magnetic resonance imaging (MRI), positron emission tomography (PET), and Near Infrared imaging agents, and for in vivo targeted delivery of therapeutics.

### BACKGROUND OF THE INVENTION

Recent years have witnessed a renaissance in fluorescence imaging driven by remarkable advances in molecular biology, and in detection technologies. The ability to discern fluorescent markers at the single molecule level is important for molecular imaging, in peculiar for monitoring signaling pathways in live cells, or for the study of cellular metabolism. Quantum dots (”Qdots” or “QDs”) represent a new crop of fluorescent agents that have many properties to facilitating their use in these studies. They are comparable in size to a protein and can be programmed to acquire biological functions. They are well tolerated by live cells, and they afford multiplexed detection due to their tunable emission. They are resistant to photobleaching, and they can be tracked at the single molecule level over extended periods of time. Thus, using biologically engineered QDs facilitates the determination of the molecular basis of certain diseases, such as cancer, and fuel the needs for finding a therapeutic treatment.

The logical extension of in vitro study at the cellular level for a disease would be to correlate the deficiency of a cell in vitro to a disease in vivo in highly structured 3D environments. In this regard, even though Qdot labeled disease sites could be extremely useful during invasive surgery and biopsy, sorely optical Qdots nanoparticles have one shortcoming: light has a penetration depth of less than a few millimeters in the best case. In vivo medical applications, such as detection of tumors or metastases, or the tracking of stem cells after cell therapy treatment require a different set of non-invasive imaging probes and techniques.

Magnetic resonance imaging is a method often used for in vivo visualization because of its infinite penetration depth and its anatomic resolution. MRI maps the relaxation processes of water protons in the sample, referred to as T₁ and T₂ relaxation times. One of the powers of MRI is its ability to extract image contrast, or a difference in image intensity between tissues, on the basis of variations in the local environment of mobile water. Unfortunately, as naturally-occurring molecules in cells lack useful fluorescence properties for imaging, intrinsic differences between tissues are often too small to provide distinguishable relaxation times. This is why exogenous contrast agents are often used, most notably in the form of small amounts of paramagnetic impurities, such as chelated Ge. The paramagnetic materials accelerate the T₁ and T₂ relaxation processes of water protons in their surroundingings. In its most elaborate version, the contrast agent is specifically targeted to tissues by chemically linking it to a targeting biomolecule. However, even in such cases, the contrast obtained in MRI might not be strong enough, because the contrast agent by itself does not provide enough sensitivity. Since increasing the local concentration of a contrast agent is not always a viable option in MRI, considerable efforts have focused in developing contrast agents with enhanced sensitivity.

The performance of a contrast agent in solution is measured by its relaxivity, defined as 1/Tᵢ˜rᵢ*[C], i=1,2, where rᵢ is the relaxivity and [C] the concentration of the contrast agent. The rule is that the higher its relaxivity, the more sensitive the contrast agent. T₁-contrast agents are agents that affect mostly the longitudinal relaxation time. They are usually made of chelated lanthanide ions and reach relaxivities of 5-30 mM⁻¹ s⁻¹. Higher relaxivities are obtained with T₂-contrast agents, i.e. agents that affect mainly the transversal relaxation time, the most prominent of which are small superparamagnetic iron oxide nanoparticles (SPIO). These particles are under heavy investigation for studying stem cells or the spatial distribution of immuno-competent cells in tumors over time. SPIO have sizes typically ranging from ˜30-50 nm in diameter. They contain thousands of iron atoms and reach relaxivities of up to 200 mM⁻¹ s⁻¹.

### SUMMARY OF THE INVENTION

In certain embodiments this invention provides probes for imaging, and/or therpeutic delivery in a variety of contexts. In certain embodiments the probes comprise a nanoparticle coated with a hydrophilic coating attached to an imaging agent. Thehydrophilic coating can comprises, e.g., one or more materials selected from the group consisting of poly(ethylene glycol) (PEG), polyethylene glycol copolymer, and silica. Any silica-containing coating described herein can comprise, e.g., SiO₂. In certain embodiments the thehydrophilic coating comprises silica. In certain embodiments thehydrophilic coating comprises poly((3-trimethoxysilyl)propyl methacrylate-r-poly(ethylene glycol) methyl ether methacrylate) (poly(TMSMA-r-PEGMA). In certain embodiments thehydrophilic coating comprises a methacrylate-based comb polymer containing pendant oligoethylene glycol side chains.

In certain embodiments the probes comprise a nanoparticle having a substantially transparent coating attached to an imaging agent. In certain embodiments the substantially transparent coating comprises silica.

In certain embodiments the probes comprise a nanoparticle attached to an MRI contrast agent where theprobe has a T₁ and/or T₂ relaxivity of greater than 200 mM⁻¹ s⁻¹ at clinical field strength. In various embodiments the probes have a T₁ and/or T₂ relaxivity of greater than about 1000 mM⁻¹ s⁻¹, greater than about 2000 mM⁻¹ s⁻¹, or greater than about 10,000 mM⁻¹ s⁻¹. In certain embodiments the nanoparticle is coated with a coating comprising silica and/or a polymer. In particular embodiments the coating comprises silica.

In any of the probes described herein, the nanoparticle can have any of a the nanoparticle can comprise an inorganic material. In certain embodiments the nanoparticle comprises a quantum dot. In certain embodiments the nanoparticle is capable of emitting in the visible region of the spectrum. In certain embodiments the nanoparticle is capable of emitting in the near infra-red region of the spectrum. In certain embodiments the nanoparticle is capable of emitting in the ultraviolet region of the spectrum. In certain embodiments the nanoparticle comprises a material selected from the group consisting of an element of Groups II-VI, a semiconductor of Groups II-VI, an oxide or nitride of the element or semiconductor of Groups II-VI.

Nanoparticles comprising an inorganic material and/or a quantam dot can, in certain embodiments, have a core having the formula MX, where:

M is one or more materials selected from the group consisting of cadmium, zinc, magnesium, mercury, aluminum, gallium, indium, and thallium; and

X is one or more materials selected from the group consisting of oxygen, sulfur, selenium, tellurium, nitrogen, phosphorus, arsenic, and antimony. For example, in particular embodiments, a nanparticle comprising an inorganic material can comprises a core and a shell, where the shell comprises a semiconductor overcoating the core. In certain embodiments the shell comprises a group II, III, IV, V, or VI semiconductor. In particular embodiments the shell comprises one or more materials selected from the group consisting of ZnO, ZnS, ZnSe, ZnTe, CdO, CdS, CdSe, CdTe, MgO, MgS, MgSe, MgTe, HgO, HgS, HgSe, HgTe, AlN, AlP, AlAs, AlSb, GaN, GaP, GaAs, GaSb, InN, InP, InAs, InSb, TlN, TlP, TlAs, and TlSb. In certain embodiments the nanoparticle comprises a CdSe core and a ZnS shell and an SiO₂ hydrophilic coating.

In any of the probes described herein, the nanoparticle has, in certain embodiments, a characteristic dimension of less than about 30 nm.

The imaging agent employed in any of the probes described herein can comprise, in particular embodiments, one or more agents selected from the group consisting of a magnetic resonance (MRI) imaging agent, a positron emission (PET) imaging agent, an electron spin resonance (ESR) imaging agent, and a near infrared (NIR) imaging agent. In certain embodiments the imaging agent comprises an MRI contrast agent comprising a material selected from the group consisting of gadolinium, xenon, iron oxide, and copper. In certain embodiments the imaging agent comprises a PET imaging agent comprising a label selected from the group consisting of ¹¹C, ¹³N, ¹⁸F, ⁶⁴Cu, ⁶⁸Ge, and ⁸²Ru. In particular embodiments the imaging agent is a PET imaging agent selected from the group consisting of [¹¹C]choline, [¹⁸F]fluorodeoxyglucose(FDG), [¹¹C]methionine, [¹¹C]choline, [¹¹C]acetate, [¹⁸F]fluorocholine, and [¹⁸F]polyethyleneglycol stilbenes.

The imaging agent can comprise, in certain embodiments, one or more agents selected from the group consisting of a cyanine derivative, and an indocyanine derivative. In particular embodiments the imaging agent comprises an agent selected from the group consisting of Cy5.5, IRDye800, indocyanine green (ICG), and an indocyanine green derivative.

The imaging agent can comprise, in certain embodiments, an electron spin resonance agent comprising a paramagnetic or superparamagnetic material. In particular embodiments the imaging agent comprises a yttrium iron garnet.

In certain embodiments the imaging agent is attached to the nanoparticle by a linker. In particular embodiments, the linker comprises a chelating agent. In particular embodiments, the linker comprises DOTA.

Any of the probes described herein can, in certain embodiments, further comprise a targeting moiety attached to the nanoparticle. In particular embodiments the targeting moiety comprises one or more moieties selected from the group consisting of a nucleic acid, a peptide, an enzyme, a lipid, an antibody, a polysaccharide, a lectin, a selectin, a sugar, an aptamers, a drug, and a receptor ligand.

In certain embodiments the targeting moiety is an antibody that binds an antigen selected from the group consisting of, a gastrointestinal cancer cell surface antigen, a lung cancer cell surface antigen, a brain tumor cell surface antigen, a glioma cell surface antigen, a breast cancer cell surface antigen, an esophageal cancer cell surface antigen, a common epithelial cancer cell surface antigen, a common sarcoma cell surface antigen, an osteosarcoma cell surface antigen, a fibrosarcoma cell surface antigen, a melanoma cell surface antigen, a gastric cancer cell surface antigen, a pancreatic cancer cell surface antigen, a colorectal cancer cell surface antigen, a urinary bladder cancer cell surface antigen, a prostatic cancer cell surface antigen, a renal cancer cell surface antigen, an ovarian cancer cell surface antigen, a testicular cancer cell surface antigen, an endometrial cancer cell surface antigen, a cervical cancer cell surface antigen, a Hodgkin's disease cell surface antigen, a lymphoma cell surface antigen, a leukemic cell surface antigen and a trophoblastic tumor cell surface antigen.

In certain embodiments the targeting moiety the targeting moiety is an antibody that binds an antigen selected from the group consisting of 5 alpha reductase, α-fetoprotein, AM-1, APC, APRIL, BAGE, β-catenin, Bc12, bcr-abl (b3a2), CA-125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, Cox-2, DCC, DcR3, E6/E7, EGFR, EMBP, Ena78, FGF8b and FGF8a, FLK-1/KDR, Folic Acid Receptor, G250, GAGE-Family, gastrin 17, GD2/GD3/GM2, GnRH, GnTV, gp100/Pme117, gp-100-in4, gp15, gp75/TRP-1, hCG, Heparanase, Her2/neu, HERS, Her4, HMTV, HLA-DR10, Hsp70, hTERT, IGFR1, IL-13R, iNOS, Ki 67, KIAA0205, K-ras, H-ras, N-ras, KSA, (CO17-1A), LDLR-FUT, MAGE Family (MAGE1, MAGE3, etc.), Mammaglobin, MAP17, Melan-A/, MART-1, mesothelin, MIC A/B, MT-MMP's, such as MMP2, MMP3, MMPI, MMP9, Mox1, MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin, PSA, PSM, RAGE-1, Rb, RCAS1, SART-1, SSX gene, family, STAT3, STn, TAG-72, TGF-α, TGF-β, and Thymosin β15, nucleolin, Ca15-3, astro Intestinal Tumor Antigen (Ca19-9), ovarian Tumor Antigen (Ca125), Tag72-4 Antigen (CA72-4) and carcinoembryonic antigen (CEA).

Any of the probes described herein that comprise a silica-coated nanoparticle can, in certain embodiments, further comprise a therapeutic moiety attached to the silica-coated nanoparticle. In certain embodiments the therapeutic moiety comprises one or more moieties selected from the group consisting of a photosensitizer, a radiosensitizer, an ESR heating moiety, an isotope, a cytotoxin, and a cancer drug. In particular embodiments the therapeutic moiety comprises an isotope selected from the group consisting of ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴¹Cu, ⁵²Fe, ^{52m}Mn, ⁵¹Cr, ¹⁸⁶, Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁷Lu, ¹⁰⁵Rh, and ¹¹¹Ag. In certain embodiments the therapeutic moiety comprises an isotope that is a gamma emitter. In certain embodiments the therapeutic moiety comprises a photosensitizer selected from the group consisting of a haematoporphyrin derivative, photophrin II, a benzoporphyrins, a tetraphenyl porphyrin, a chlorine, and a phthalocyanine.

Another aspect of the invention is the use of any probe described herein in the manufacture of a medicament for the detection and/or treatment of a cancer.

In another aspect, the invention provides a method of making a nanoprobe, the method comprising:

forming a silica shell around a nanoparticle;

chelating a paramagnetic or superparamagnetic compound; and

coupling the chelated compound to the silica shell thereby forming a nanoprobe. In certain embodiments the method further comprises attaching a targeting moiety to the nanoprobe. In certain embodiments the method further comprises attaching a therapeutic moiety to the nanoparticle.

The invention also provides a method of detecting a cancer cell, themethod comprising:

contacting the cell with a probe comprising a nanoparticle coated with a hydrophilic coating attached to a targeting moiety and an imaging agent, whereby theprobe preferentially associates with a cancer cell; and

detecting the imaging agent thereby providing an indication of the presence and/or location of the cancer cell. In certain embodiments the contacting comprises a modality selected from the group consisting of systemic administration to a mammal, local administration to a tumor or tumor site, administration to a surgical site, ex vivo administration to a sample, and in situ administration to a histological preparation. In various embodiments the cancer cell is: a cell in a solid tumor, a metastatic cell, a cell present in a human and/or a cell present in a non-human mammal. In certain embodiments the nanoparticle coated with a hydrophilic coating attached to a targeting moiety and an imaging agent comprises any such nanoparticles described herein. More specifically, the nanoparticle itself can comprise any nanoparticle described herein, the hydrophilic coating can comprise any hydrophilic coating described herein, the targeting moiety can comprise any targeting moiety described herein, and the imaging agent can comprise any imaging agent described herein.

In certain embodiments the method of detecting a cancer cell employs a probe that further comprises a therapeutic moiety attached to a silica-coated nanoparticle. In particular embodiments thetherapeutic moiety comprises one or more moieties selected from the group consisting of a photosensitizer, a radiosensitizer, an ESR heating moiety, an isotope, a cytotoxin, and a cancer drug.

Another aspect of the invention is a method of inhibiting the growth and/or proliferation of a cancer cell, the method comprising:

contacting the cell with a probe comprising a nanoparticle coated with a hydrophilic coating attached to a targeting moiety, an imaging agent, and a therapeutic moiety whereby the probe preferentially associates with a cancer cell and inhibits the growth and/or proliferation of the cell. In certain embodiments the contacting comprises a modality selected from the group consisting of systemic administration to a mammal, local administration to a tumor or tumor site, and administration to a surgical site. In particular embodiments the contacting comprises administering the probe to a mammal via a modality selected from the group consisting of oral administration, nasal administration, topical administration, transdermal administration, rectal administration, systemic administration, and administration directly to a tumor or tumor site. In various embodiments the cancer cell is: a cell in a solid tumor, a metastatic cell, a cell present in a human and/or a cell present in a non-human mammal. In certain embodiments the nanoparticle coated with a hydrophilic coating attached to a targeting moiety, an imaging agent, and a therapeutic moiety comprises any such nanoparticles described herein. More specifically, the nanoparticle itself can comprise any nanoparticle described herein, the hydrophilic coating can comprise any hydrophilic coating described herein, the targeting moiety can comprise any targeting moiety described herein, the imaging agent can comprise any imaging agent described herein, and the therapeutic moiety can comprise any therapeutic moiety described herein.

In certain embodiments the invention provides a multimodal probe comprised of a water soluble, silica-coated nanoparticle exhibiting an imaging agent, targeting agent and a therapeutic agent. In particular embodiments the nanoparticle comprises an inorganic core embedded into an ultra-thin silica shell, where the inorganic core is comprised of semiconductor material elements of Groups II-VI. The inorganic core of the nanoparticle can, in certain embodiments, comprise a CdSe core and a ZnS shell which further comprises a SiO₂ hydrophilic coating. In particular embodiments the nanoparticle is linked to the imaging agent, targeting agent and therapeutic agent by a linking agent. The linking agent can, for example, be a chelated paramagnetic ion or labeled chelator, a heterobifunctional crosslinker, functional groups, affinity agents, stabilizing groups, and combinations thereof. In certain embodiments the imaging agent is an MRI, PET or deep tissue Near Infrared (NIR) imaging agent. In particular embodiments the imaging agent is an MRI imaging agent selected from the group consisting of gadolinium, xenon, iron oxide, copper, Gd³⁺-DOTA, and ⁶⁴Cu²⁺-DOTA. In particular embodiments the imaging agent is a PET imaging agent selected from the group consisting of [¹¹C]choline, [¹⁸F]fluorodeoxyglucose (FDG), [¹¹C]methionine, [¹¹C]choline, [¹¹C]acetate, [¹⁸F]fluorocholine, and other radionuclides labeled with ⁶⁴Cu or ⁶⁸Ge. The targeting agent can, in certain embodiments, be selected from the group consisting of nucleic acids, oligonucleotides, peptides, proteins, enzymes, lipids, antibodies, polysaccharides, lectins, selectins, and small molecules such as sugars, aptamers, drugs, and ligands. For example, the targeting agent can be an antibody or a signaling peptide. In certain embodiments the therapeutic agent is selected from the group consisting of: nucleic acids (both monomeric and oligomeric), peptides, proteins, enzymes, lipids, antibodies, polysaccharides, lectins, selectins, and small molecules such as sugars, aptamers, drugs, and ligands. For example, the therapeutic agent can be an antibody, drug or photosensitizer.

In particular embodiments the invention provides a multimodal probe for in vivo imaging and therapy that, (1) detects diseased cells by MRI, PET or deep tissue Near Infrared (NIR) imaging, and is capable of detecting diseased cells with greater sensitivity than is possible with existing technologies, (2) targets molecules that localize to normal or diseased cells, and (3) initiates apoptosis of diseased cells.

The invention also provides a nanoparticle-based technology platform for multimodal cancer imaging and therapy that, (1) detects cancer by MRI, ESR, PET, or deep tissue Near Infrared (NIR) imaging, and is capable of detecting cancer cells with greater sensitivity than is possible with existing technologies, (2) targets molecules that localize on the surface of cancer cells, and (3) initiates apoptosis of cancer cells by local infrared laser-mediated photodynamic therapy (PDT).

Further, the invention provides a of increasing the relaxivity of an NMR, MRI, PET, or ESR imaging agent, the method comprising coupling the agent to a a nanoparticle. In particular embodiments the nanoparticle is coated with a coating comprising silica.

The present invention provides water soluble, silica-coated nanoparticles as multimodal probes exhibiting an imaging agent, targeting agent and a therapeutic agent. The multimodal probes are constructed upon an inorganic core embedded into an ultra-thin silica shell linked to the imaging agent, targeting agent and therapeutic agent.

It is an object of the invention to provide a nanoparticle-based technology platform for multimodal in vivo imaging and therapy that, (1) detects diseased cells by MRI, PET and deep tissue Near Infrared (NIR) imaging, and is capable of detecting diseased cells with greater sensitivity than is possible with existing technologies, (2) targets molecules that localize to normal or diseased cells, and (3) initiates apoptosis of diseased cells.

It is another object of the invention to provide a nanoparticle-based technology platform for multimodal cancer imaging and therapy that, (1) detects cancer by MRI, PET and deep tissue Near Infrared (NIR) imaging, and is capable of detecting cancer cells with greater sensitivity than is possible with existing technologies, (2) targets molecules that localize on the surface of cancer cells, and (3) initiates apoptosis of cancer cells by local infrared laser-mediated photodynamic therapy (PDT).

The present multimodal probes may be used in cancer detection and treatment, and contemplated for use in cancers such as prostate, breast, brain, and epithelial cancers.

The present invention further provides methods and uses for the present probes for detection, imaging, and treatment of other diseases in vivo with the use of a single probe, such as diseases characterized by inflammation, cardiovascular or neurological diseases.

### Definitions

The term “cancer markers” refers to biomolecules such as proteins that are useful in the diagnosis and prognosis of cancer. As used herein, “cancer markers” include but are not limited to: PSA, human chorionic gonadotropin, alpha-fetoprotein, carcinoembryonic antigen, cancer antigen (CA) 125, CA 15-3, CD20, CDH13, CD 31,CD34, CD105, CD146, D16S422HER-2, phospatidylinositol 3-kinase (PI 3-kinase), trypsin, trypsin-1 complexed with alpha(1)-antitrypsin, estrogen receptor, progesterone receptor, c-erbB-2, be 1-2, S-phase fraction (SPF), p185erbB-2, low-affinity insulin like growth factor-binding protein, urinary tissue factor, vascular endothelial growth factor, epidermal growth factor, epidermal growth factor receptor, apoptosis proteins (p53, Ki67), factor VIII, adhesion proteins (CD-44, sialyl-TN, blood group A, bacterial lacZ, human placental alkaline phosphatase (ALP), alpha-difluoromethylornithine (DFMO), thymidine phosphorylase (dTHdPase), thrombomodulin, laminin receptor, fibronectin, anticyclins, anticyclin A, B, or E, proliferation associated nuclear antigen, lectin UEA-1, cea, 16, and von Willebrand's factor.

The terms “ligand” or “binding moiety”, as used herein, refers generally to a molecule that binds to a a particular target molecule and forms a bound complex as described above. The binding can be highly specific binding, however, in certain embodiments, the binding of an individual ligand to the target molecule can be with relatively low affinity and/or specificity. The ligand and its corresponding target molecule form a specific binding pair. Examples include, but are not limited to small organic molecules, sugars, lectins, nucleic acids, proteins, antibodies, cytokines, receptor proteins, growth factors, nucleic acid binding proteins and the like which specifically bind desired target molecules, target collections of molecules, target receptors, target cells, and the like.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv′s (scFv), however, alternative expression strategies have also been successful. For example Fab molecules can be displayed on phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage (e.g., scFv, Fv, Fab and disulfide linked Fv (Reiter et al. (1995) Protein Eng. 8: 1323-1331).

The term “specifically binds”, as used herein, when referring to a SHAL or to a biomolecule (e.g., protein, nucleic acid, antibody, etc.), refers to a binding reaction that is determinative of the presence of the SHAL or biomolecule in a heterogeneous population of molecules (e.g., proteins and other biologics). Thus, under designated conditions (e.g. binding assay conditions in the case of a SHAL or stringent hybridization conditions in the case of a nucleic acid), the specified ligand or SHAL preferentially binds to its particular “target” molecule and preferentially does not bind in a significant amount to other molecules present in the sample.

An “effector” refers to any molecule or combination of molecules whose activity it is desired to deliver/into and/or localize at a target (e.g. at a cell displaying a characteristic marker). Effectors include, but are not limited to labels, cytotoxins, enzymes, growth factors, transcription factors, drugs, lipids, liposomes, etc.

The term “anti-cancer drug” is used herein to refer to one or a combination of drugs conventionally used to treat cancer. Such drugs are well known to those of skill in the art and include, but are not limited to doxirubicin, vinblastine, vincristine, taxol, etc.

The term “nanoparticle” refers to a particle having a sub-micron (μm) size. In various embodiments, microparticles have a characteristic size (e.g., diameter) less than about 1 μm, 800 nm, or 500 nm, preferably less than about 400 nm, 300 nm, or 200 nm, more preferably about 100 nm or less, about 50 nm or less or about 30 or 20 nm or less.

### BRIEF DESCRIPTION OF THE DRAWINGS

FIG. 1A shows a a schematic of an illustrative embodiment of the multimodal probes, while FIG. 1B shows a scheme for the preparation of the paramagnetic probes. Gadolinium chloride is reacted with a DOTA complex under controlled acidic conditions at 80° C. Gd(III) is stabilized inside DOTA by hydrogen bonds with the carboxylic groups (dash line). Subsequently, the amine group, which does not take part in Gd(III) complexion, is converted into a maleimide group which will react with thiols on the silica-coated nanoparticles. The payload of GdDOTA per nanoparticle depends on the surface area. In certain embodiments, it can vary from ˜50 up to several hundreds. Due to this coupling scheme, Gd(III) is stably chelated in PBS buffer over periods exceeding a few months.

FIG. 2 shows spin-lattice (T₁) relaxation MRI images (T₁-weighted images) were taken using a fast imaging with steady-state procession (FISP) with inversion recovery (IR) sequence of phosphate buffer, SiO2-coated QDs (4 μM), DOTA (no Gd) attached to SiO₂-coated QDs (4 μM), Gd-DOTA attached to SiO₂-coated QDs (4 μM), and Gd-DOTA with various concentrations from 0.39 to 7.35 mM at ¹H resonance frequency of 400 MHz. All data was taken at room temperature.

FIG. 3 shows spin-spin (T₂) relaxation MRI images (T₂-weighted images) were taken using a multislice multiecho (MSME) sequence of phosphate buffer, SiO₂-coated QDs (4 μM), DOTA (no Gd) attached to SiO₂-coated QDs (4 μM), Gd-DOTA attached to SiO₂-coated QDs (4 μM), and Gd-DOTA with various concentrations from 0.39 to 7.35 mM at a ¹H resonance frequency of 400 MHz. All data was taken at room temperature.

FIG. 4A shows T₁ and T₂ MRI maps taken at various concentrations of Gd-DOTA attached to SiO₂-coated QDs ranging from 0.125 to 4 μM at 400 MHz. This data was taken at room temperature. During the same experiment, Gd-DOTA at a Gd³⁺ concentration of 390 μM and a phosphate buffer were imaged as a reference control. FIG. 4B: Plots of 1/T₁ and 1/T₂ vs nanoparticle concentration are shown for the same samples. The slopes represent the relaxivity and correspond to r₁=808±15 mM⁻¹s⁻¹ and r2=3003.5±57.1 mM⁻¹s⁻¹. Relaxivity values per Gd ion are r₁=18±0.3 mM⁻¹s⁻¹ and r₂=67±1.3 mM⁻¹s⁻¹. The relaxivity per Gd ion is shown in brackets.

FIG. 5 shows relaxivities of Gd-DOTA attached to SiO₂-coated QDs as a function of the 1H resonance frequency. The lines are guides for the eyes. The trend is similar to that of unbound Gd-DOTA. Notice, however, how the T₂ relaxivity is always larger than the T₁ relaxivity.

FIG. 6 shows relaxivity of 10-nm Au colloids coated with a silica shell at 20 MHz. The total size of the particles (including the silica shell) is about 15-18 nm. The particles contain more than 300 Gd-DOTA, thereby reaching relaxivity values close to those of organic dendrimers of generation N=7.

FIG. 7 shows an axial T₁-weighted image of the bladder of a mouse, before and after injection of Gd-DOTA attached to SiO₂-coated QDs. The body of the mouse is highlighted by the dashed white line in the left image. Features outside this line correspond to catheters and heating pad channels. The bladder is shown at the top-center of the mouse. Five minutes after injection, nanoparticles start to accumulate in the bladder (right image, arrow). Notice how the contrast comes from only the lower part of the bladder, possibly indicating the sedimentation of the nanoparticles in the bladder.

FIG. 8 is a photograph of electrophoresis of DOTA coated Qdots showing an increase in size of the multimodal particles.

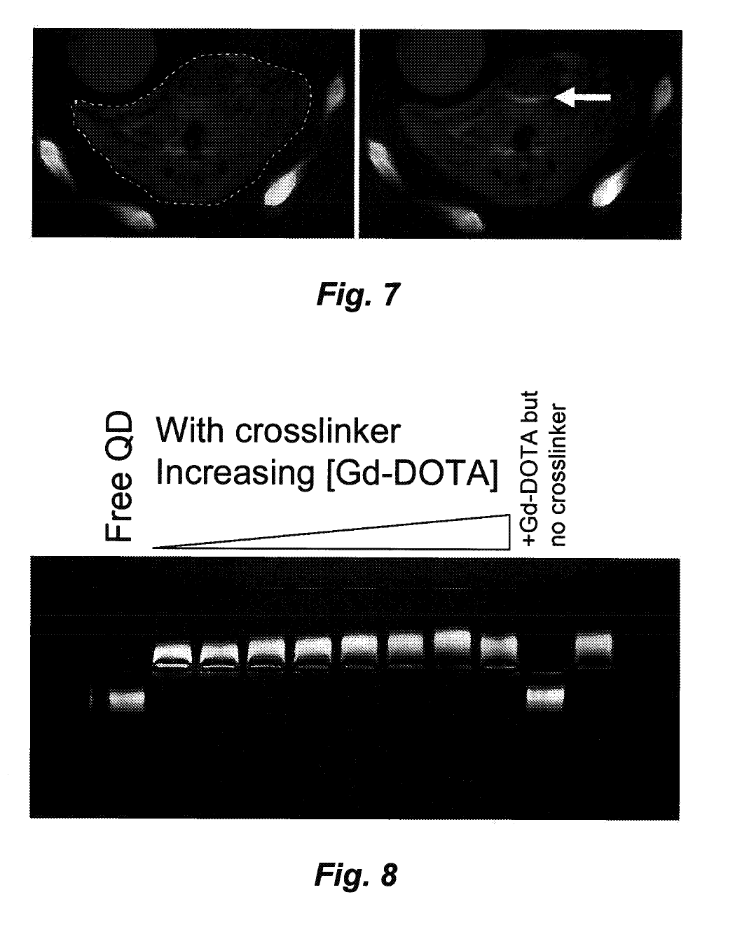

FIG. 9, panels A-C, show that antibody conjugated Qdots are internalized by cancer cells. Panel A: Left, antibody-nanocrystal conjugates, Right, nanocrystal only. Panel B: Only antibody-nanocrystal conjugates are internalized by BT474 tumor cells. Panel C: live cell image of internalized nanocrystal.

FIG. 10 shows that targeting to breast cancer cells using anti-Her2 single chain antibody conjugated to Gd-DOTA-Qdot. Cells were incubated for 30 min with the nanoprobes and washed before fluorescent imaging.

### DETAILED DESCRIPTION

In certain embodiments, this invention pertains to nanoparticle-based probes that are useful as imaging (e.g., contrast) agents, and/or therapeutics. In various embodiments the nanoparticle-based probes are are effective a multiple-modality effectors. That is, they can simultaneously provide one or more imaging modalities, and/or one or more targeting modalitizes, and/or one or more therapeutic modalities.

It was a surprising discovery that attaching, for example, magnetic resonance imaging materials, to a nanoparticle substantially increases the T₁ and/or T₂ relaxivity of the imaging material. Thus, for example, probe constructs are described herein have T₁ and T₂ relaxivities of up to ˜13,000 and ˜15,000 mM⁻¹ s⁻¹ at clinical fields with a size of only ˜15-20 nm. Smaller constructs of size in the range of ˜8-10 nm are also provided that exhibit T₁ and T₂ relaxivities of ˜1000 and 2000 mM⁻¹ s⁻¹. Accordingly, methods of increasing relaxivity of a material are provided where the methods involve coupling the material to a nanoparticle.

Thus, in certain embodiments nanoparticle-based imaging probes are provided comprising a nanoparticle attached to one or more imaging materials (e.g., contrast agents). Suitable contrast agents include, but are not limited to magnetic resonance imaging materials, electron spin resonance (ESR) materials, near infrared materials, PET materials, and the like. The nanoparticle can itself be a moiety that provides a detectable signal (e.g., a quantum dot) in which case the nanoparticle/agent combination can provide at least two different detection modalities.

In certain embodiments the nanoparticle can be coated with a coating that improves water solubility (e.g., a hydrophilic coating) of the particle and/or serum halflife. The coating can be substantially transparent or translucent to facilitate the emission not an optical signal, when the nanoparticle is for example fluorescent. Thus, for example, in certain embodiments, the nanopartilce is covered with a coating comprising silica and/or a polymer (e.g., poly(ethylene glycol), etc.

While, in certain embodiments, probes are provided comprising simply a nanoparticle (e.g., coated or uncoated) attached to an imaging agent (e.g., MRI imaging agent), in certain other embodiments, additional functionality can be afforded by coupling other agents to the nanoparticle. Thus certain probes additionally comprise a targeting moiety attached to the nanoparticle to afford preferential and/or selective delivery and/or internalization by a target cell or tissue (e.g., a tumor cell or tumor mass). Various targeting moieties include, but are not limited to antibodies, receptor ligands, signal peptides, and the like.

In various embodiments the nanoparticle can additionally or alternatively have one or more therapeutic moieties attached thereto and thereby offer a treatment/therapetutic modality in addition to the detection modalities. Therapeutic moieties include, but are not limited to nucleic acids, photodynamic agents, ESR heating agents, radionuclides, ribozymes, antisense molecules, RNAi, and pharmaceuticals.

In certain embodiments the probes of this invention can simply be used as detection agents (e.g., as MRI contrast agents). When coupled to a targeting moiety they can, for example, be used to detect the presence, and/or location, and/or size of the target (e.g., a tumor cell or tumor mass) in vivo and/or in vitro (e.g., in a biological sample). In certain embodiments the probes are used simply as therapeutic agents that, when coupled to a targeting moiety, can be used to deliver a therpeutic moiety to a target cell or tissue. In certain embodiments the probes are used both to image a target cell or tissue and to deliver one or more therapeutic moieties thereto.

Thus, in certain embodiments methods are provided for imaging (e.g., detecting or quantifying the presence or absence, and/or the location and/or the size of a target) cell and/or tissue. Similarly, in certain embodiments methods are provided for delivering a therapeutic moiety in proximity to, and/or on the surface of, and/or internalized into a target cell and/or tissue. In certain embodiments the methods involve using the nanoparticle probe to both image a target cell or tissue and to deliver a theraputic moiety thereto.

In one illustrative embodiment, multimodal probes are provided comprising water soluble, silica-coated nanoparticles suitable for imaging, and/or targeting and/or therapeutics. In certain embodiments the nanoparticles comprise an inorganic core embedded into an ultra-thin silica shell exhibiting or linked to an imaging agent, and/or targeting agent, and/or therapeutic agent. In a certain illustrative embodiments, the nanoparticle is comprised of a silica-coated inorganic core particle, densely coated with the imaging agent, and/or targeting agent, and/or therapeutic agent. In one specific illustrative embodiment the probe comprises a paramagnetic nanoprobe of about 2 nm to about 100 nm, preferably about 5 nm to about 50 nm or 25 nm, more preferably about 10 nm to about 15 nm or 10 nm in diameter that consists in an inner inorganic particle protected with an ultra-thin silica shell to which several chelated paramagnetic ions are covalently linked.

In certain embodiments the probes describe herein provide a nanoparticle-based technology platform for multimodal in vivo and/or ex vivo imaging and therapy.

Thus, various probes described herein allow detection and/or localization and/or size determination of diseased cells and/or tissues by MRI, PET, ESR, and/or deep tissue Near Infrared (NIR) imaging, facilitate detection of diseased cells with greater sensitivity than is possible with existing technologies. In addition, the same probes can be used to target therapeutic moieties to to normal or diseased cells, and/or initiates apoptosis of diseased cells.

### I. Nanoparticles.

In certain embodiments the probes described herein comprise nanoparticles (i.e., a particle whose size is typically less than about 1,000 nm, preferably less than abut 800 nm, more preferably less than about 500 nm or less than about 300 nm, or less than about 200 nm, 150 nm, or 100 nm.

Suitable nanoparticles include, for example semiconductor nanocrystals, metal nanocrystals, hollow nanoparticles, carbon nanospheres, nanorods, nanofibers, nanotubes, nanotori, and the like. In certain embodiments the nanoparticles typically have a diameter in the range of about 1 nm to about 100 nm, preferably from about 1 nm to about 50 nm, more preferably from about 1 nm to about 30 nm, or 20 nm, more preferably less than about 18 nm, 16 nm, 14 nm, 12 nm, 10 nm, or 8 nm. The nanoparticles can be of any shape including, rods, arrows, teardrops, nanocrescents, and tetrapods (see, e.g., Alivisatos et al. (2000) J. Am. Chem. Soc. 122:12700-12706). Other suitable shapes include, but are not limited to square, round, elliptical, triangular, rectangular, rhombal and the like. In certain embodiments the nanoparticle are homogenous, while in other embodiments, the material properties of the nanoparticle are anisotropic.

In certain embodiments the nanoparticles typically comprise a shell and a core. In illustrative embodiments, the shell material, when present, can be selected to provide a bandgap energy that is greater than the bandgap energy of the core material. In some embodiments, the shell material can have an atomic spacing close to that of the core material.

In certain embodiments the nanoparticle comprise a quantum dot. The terms “quantum dot”, “Qdot”, and “QD” refer to a nanoparticle typically capable of emitting electromagnetic radiation (i.e., a signal) upon excitation by an energy source. In various embodiments quantum dots include metal colloidal nanoparticles, and/or luminescent metal or semiconductor nanocrystals, e.g., nanoparticles typically comprising a core and a shell structure.

The nanoparticle portion of the conjugates described herein typically comprise a core and a shell. The core and the shell may comprise the same material or different materials. The shell may further comprise a hydrophilic coating or another group that facilitates conjugation of a chemical or biological agent or moiety to a nanoparticle (i.e., via a linking agent). In some embodiments, the semiconductor nanocrystals comprise a core upon which a hydrophilic coating has been deposited.

The core and the shell may comprise, e.g., an inorganic semiconductive material, a mixture or solid solution of inorganic semiconductive materials, or an organic semiconductive material. Suitable materials for the core and/or shell include, but are not limited to semiconductor materials, carbon, metals, and metal oxides. In a preferred embodiment, the nanoparticles comprise a semiconductor nanocrystal. In a particularly preferred embodiment, the semiconductor nanocrystals comprise a CdSe core and a ZnS shell which further comprises a SiO₂ hydrophilic coating.

The core typically has a diameter of about 1, 2, 3, 4, 5, 6, 7, or 8 nm. The shell typically has thickness of about 1, 2, 3, 4, 5, 6, 7, or 8 nm and a diameter of about 1 to about 10, 2 to about 9, or about 3 to about 8 nm. In a preferred embodiment, the core is about 2 to about 3 nm in diameter and the shell is about 1 to about 2 nm in thickness.

Suitable semiconductor materials for the core and/or shell include, but are not limited to, elements of Groups II-VI (ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, InAs, InSb, and the like) and IV (Ge, Si, and the like), and alloys or mixtures thereof. Suitable metals and metal oxides for the core and/or shell include, but are not limited to, Au, Ag, Co, Ni, Fe₂O₃, TiO₂, and the like. Suitable carbon nanoparticles include, but are not limited to, carbon nanspheres, carbon nano-onions, and fullerenes. In certain embodiments, gold nanoparticles are provided as the core particle.

In certain embodiments illustrative embodiments, the nanoparticle (quantum dot) has a core-shell structure comprising a core comprising a semiconductor material with an overcoating. In certain embodiments the overcoating can be a semiconductor material having a composition different from the composition of the core. Certain preferred quantum dot nanoparticles photoluminesce and have high emission quantum efficiencies.

In certain embodiments nanoparticle includes a core having the formula MX, where M is cadmium, zinc, magnesium, mercury, aluminum, gallium, indium, thallium, or mixtures thereof, and X is oxygen, sulfur, selenium, tellurium, nitrogen, phosphorus, arsenic, antimony, or mixtures thereof. An M-containing salt can be the source of M in the nanoparticle, while an X-containing compound can be the source of the X in the nanoparticle. In certain embodiments an M -containing salt provides a safe, inexpensive starting material for manufacturing the nanoparticle relative to typical organometallic reagents which can be air sensitive, pyrophoric, or volatile. In certain embodiments the M-containing salt is not air sensitive, is not pyrophoric, and is not volatile relative to organometallic reagents.

In certain embodiments the nanoparticle is formed by a method, described in U.S. Pat. No. 6,576,201, that includes contacting a metal, M, or an M-containing salt, and a reducing agent to form an M-containing precursor, M being Cd, Zn, Mg, Hg, Al, Ga, In or Tl. The M-containing precursor is contacted with an X donor, X being O, S, Se, Te, N, P, As, or Sb. The mixture is then heated in the presence of an amine to form the nanoparticle. In certain embodiments, heating can take place in the presence of a coordinating solvent. In certain embodiments the core nanoparticle is overcoated to form a shell. The overcoating can be accomplished by contacting a core nanoparticle population with an M-containing salt, an X donor, and an amine, and forming an overcoating having the formula MX on a surface of the core (e.g., as described in U.S. Pat. No. 6,576,201). In certain embodiments, a coordinating solvent can be present. In certain embodiments the amine can be a primary amine, for example, a C₈-C₂₀ alkyl amine. The reducing agent can be a mild reducing agent capable of reducing the M of the M-containing salt. Suitable reducing agents include, but are not limited to a 1,2-diol or an aldehyde. In certain embodiments the 1,2-diol can be a C₆-C₂₀ alkyl diol. In certain embodiments the aldehyde can be a C₆-C₂₀ aldehyde. In certain illustrative embodiments the M-containing salt can include, but is not limited to a halide, carboxylate, carbonate, hydroxide, or diketonate. The X donor can include, but is not limited to a phosphine chalcogenide, a bis(silyl)chalcogenide, dioxygen, an ammonium salt, or a tris(silyl)pnictide.

In various embodiments the overcoating, when present, is a semiconductor material. Illustrative semiconductor materials include, but are not limited to group II VI, III V or IV semiconductor, such as, for example, ZnO, ZnS, ZnSe, ZnTe, CdO, CdS, CdSe, CdTe, MgO, MgS, MgSe, MgTe, HgO, HgS, HgSe, HgTe, AlN, AlP, AlAs, AlSb, GaN, GaP, GaAs, GaSb, InN, InP, InAs, InSb, TlN, TIP, TlAs, TlSb, or mixtures thereof.

In certain embodiments the nanoparticle comprises a tellurium containing nanoparticle that is capable of emitting in the near infrared region of the spectrum. The manufacture of tellurium-containing nanoparticles is described, for example in U.S. Pat. Nos. 6,607,829; and 6,322,901, and by Rajh et al. (1993) J. Phys. Chem. (97):11999-12003, and the like.

The foregoing nanoparticles and synthesis methods are intended to be illustrative and not limiting. Semiconductor nanoparticles can be made using any method known in the art. For example, methods for synthesizing semiconductor nanocrystals comprising Group III-V semiconductors or Group II-VI semiconductors are set forth in, e.g., U.S. Pat. Nos. 5,751,018 ; 5,505,928; and 5,262,357. The size of the semiconductor nanocrystals can be controlled during formation using crystal growth terminators U.S. Pat. Nos. 5,751,018 ; 5,505,928; and 5,262,357. Methods for making semiconductor nanoparticles are also set forth in Gerion et al. (2001) J. Phys. Chem. 105(37):8861-8871, and Peng et al., (1997) J. Amer. Chem. Soc., 119(30): 7019-7029, and the like.

The absorption and emission properties of semiconductor nanocrystal nanoparticles offer several advantages over dye molecules which have narrow wavelength bands of absorption (e.g., about 30-50 nm), broad wavelength bands of emission (e.g., about 100 nm), and broad tails of emission (e.g., another 100 nm) on the red side of the spectrum. These properties of dyes impair the ability to use a plurality of differently colored dyes when exposed to the same energy source.

In contrast, the semiconductor nanocrystal nanoparticles are capable of absorbing and emitting radiation (i.e., luminescing) in response to a broad range of wavelengths, including the range from gamma radiation to microwave radiation. The semiconductor nanocrystals are also capable of emitting radiation within a narrow wavelength band of about 50, 40, 30, 20, or 10 nm or less. Thus, a single energy source can be used to excite the luminescence of a plurality of semiconductor nanocrystals, each of which comprise a different material. The plurality of semiconductor nanocrystals can easily be distinguished following excitation because each semiconductor nanocrystal will emit only a narrow wavelength band.

The wavelength band emitted from the semiconductor nanocrystal is related to the physical properties (e.g., size, shape, and material), of the semiconductor nanoparticle. More particularly, the wavelength band emitted by the semiconductor nanoparticles can be affected by (1) the size of the core; (2) the size of the core and the size of the shell; (3) the composition of the core and shell. For example, a semiconductor nanocrystal comprised of a 3 nm core of CdSe and a 2 nm thick shell of CdS will emit a narrow wavelength band of light with a peak intensity wavelength of 600 nm. In contrast, a semiconductor nanocrystal comprised of a 3 nm core of CdSe and a 2 nm thick shell of ZnS will emit a narrow wavelength band of light with a peak intensity wavelength of 560 nm. As another example, when a 1-10 monolayer thick shell of CdS is epitaxially grown over a core of CdSe, there is a dramatic increase in the room temperature photoluminescence quantum yield. Thus, one of skill in the art will appreciate that any of the physical properties of the semiconductor nanocrystals can be modified to control the wavelength band of the semiconductor nanoparticle and the corresponding nanoparticle/imaging agent/targeting moiety/therapeutic moiety conjugate.

Thus, one of skill in the art will appreciate that a number of variables can be routinely adjusted to selectively manipulate wavelength band emitted by the semiconductor nanocrystals. For example, the composition of the semiconductor nanocrystal core or shells can be varied and the number of shells around the core of the semiconductor nanocrystal can be varied. In addition, semiconductor nanocrystals comprising different core materials, but the same shell material can be synthesized. Semiconductor nanocrystals comprising the same core material, but the different shell materials can also be synthesized.

Recently, nanoparticles have also been shown to absorb and emit light directly in the visible region as a multiple photon process. This involves the simultaneous excitation by two or more lower energy photons to reach the same excited energy state that can also be reached by a single higher energy photon. The ability for a chromophore to absorb two photons is dependent on the parameters shown in equation 1, where “δ” is the two photon cross section and “I” is the intensity of light.

Rate=1/2(δ)<I²> (1)

The cross sectional area is the two photon equivalent of the single photon absorption extinction coefficient, and is a quantitative measure of the ability of the chromophore to absorb two photons simultaneously. Qdots have been shown to be an ideal multiphoton fluorophone because of their cross-section. For CdSe Qdots (4.5 nm diameter), this was measured to be 47,000 Goeppert-Mayer units (GM) which is an order of magnitude higher than most organic two photon absorbers. The multiphoton excitation of Qdots can avoid interference from absorption of light by tissue

II. Hydrophilic coating.

In certain embodiments the nanoparticle can be covered with a hydrophilic coating e.g., any compound with an affinity for aqueous materials such as H₂O and/or stabilizing groups to enhance the solubility of the nanoparticles in an aqueous solution and/or to increase serum half-life in vivo. Illustrative hydrophilic coatings include, but are not limited to SiO, SiO₂, polyethylene glycol, ether, mecapto acid and hydrocarbonic acid, dihydroxylipoic acid (DHLA), various hydrophilic polymers (e.g., polyethylene glycol ether), and the like. Suitable stabilizing groups include, e.g. positively or negatively charged groups or groups that facilitate steric repulsion. In one illustrative embodiment, the hydrophilic coating is a silica shell (e.g., comprising SiO₂). In certain embodiments the the hydrophilic coating is about 0.1, 0.25, 0.5, 0.75, 1, 2, 3, 4, 5, 6, 7, 8, 9, 10, 11, 12, 131, 14, 15, 16, 17, 18, 19, or 20 nm thick. Methods of silanizing semiconductor nanocrystals are well known in the art and are described in, e.g., Gerion et al., Chemistry of Materials, 14:2113-2119 (2002). Other methods for generating water-soluble semiconductor nanocrystals are described in, e.g., Mattoussi et al. (2001) Physica Status Solidi B, 224 (1):277-283, and Chan et al. (1998) Science, 281:2016-2018 (1998). Similarly, coating of nanoparticles with poly((3-trimethoxysilyl)propyl methacrylate-r-poly(ethylene glycol) methyl ether methacrylate) (poly(TMSMA-r-PEGMA) is described by Jon et al. (2003) Langmuir 19: 9989-9993, and coating of nanoparticles with methacrylate-based comb polymer containing pendant oligoethylene glycol side chains is described by Hyun et al. (2003) 15: 576-579.

In certain embodiments illustrative embodiments, the hydrophilic coating comprises a silica shell having a thickness of about 0.5 to about 5, about 1 to about 4, or about 2 to about 3 nm. In various embodiments the silica shell is amorphous and porous. Silica shells can be deposited on the core or the shell of the semiconductor nanocrystal using the methods described in, e.g., Alivisatos et al. (1998) Science 281: 2013-2016 and Gerion, et al., (2001) J. Phys. Chem. 105(37): 8861-8871. In one illustrative embodiment the semiconductor nanocrystals have core/shell configuration of CdSe/ZnS/SiO₂ where the layers are about 25/5/50 Å respectively from the center of the core.

In certain embodiments, an ultrathin paramagnetic silica shell is grown around nanocrystals of different nature. One illustrative procedure to embed, for example, gold colloids of 5 nm and 10 nm diameter into a thin silica shell is described herein. In addition, the synthesis of silica shells around Au cores has been detailed in Liz-Marzan et al. (1996) Langmuir 12(18): 4329-4335, which describes a 15 nm Au seed and shows how to grow thick shells (up to >80 nm) over a period of several days.

Two main concerns in growing a silica shells around, e.g., Au seeds are the avoidance of cross-linking between nanoparticles and the control of the polymerization rate. The latter is facilitated by the use of an anhydrous solvent, while the former is facilitated by dilute solutions of nanoparticles. This is because polycondensation of methoxysilane into siloxane bonds is driven by hydrolysis and heat and basicity. Accordingly, synthesis is facilitated by avoiding both.

Controlled polymerization is facilitated by anhydrous solvents because citrate-stabilized Au colloids are poorly soluble in solvents other than water (including aqueous buffers). A dilute solution of nanoparticle is readily achieved, for example, by diluting 20 ml of as-purchased 5 nm Au colloids (83 nM) in more than 500 mL of water to start with published protocols.

In an improved method described herein, permits the formation of silanized Au colloids in small volumes (<1-3 ml) at high nanoparticle concentration (>1 μM for 5 nm Au). The methods are is amenable to an easy scale-up and are applicable to the silanization of other inorganic cores such as iron oxide and the like.

Thus, in certain embodiments the silanization protocol for Au (or other) colloids calls for an exchange of the citrate capping ligands with a phosphine stabilizer (Bis(p-sulfonatophenyl)phenylphosphine), as described by Zanchet et al. (2001) Nano Lett. 1:32-35; and Loweth et al. (1999) Angewandte Chemie International Edition 38 (12): 1808-1812. Phosphine-stabilized Au colloids are soluble in buffers and water at concentrations ˜50-100-fold higher than the original ones. As described herein in the Examples, nanoparticles were silanized at these high concentrations.

The phosphine capping is an intermediate step to facilitate manipulation of Au (or other) colloids in water while preventing their aggregation. To grow the silica shell, phosphine groups are replaced with thiolate primers, specifically mercaptopropyltrimethoxysilane or MPS. Because of the strong affinity between thiols and gold surfaces, the capping exchange is fast (<20 min) and efficient. The methoxysilane or silanol groups of MPS act as an anchor molecule pon which the silica shell forms. The consolidation and polymerization of MPS into a siloxane or silica shell can be controlled by choosing weakly alkaline aqueous solutions (pH˜7.5-8) instead of heat. While the shell is slowly forming, fresh MPS and PEG-siloxane can be incorporated into the shell. The shell growth can be finally quenched by converting the remaining silanol groups into unreactive methyl groups. At this point silanized Au colloids can be purified from excess silane by, for example, dialysis, repeated runs in centricon 100 devices, and size-exclusion column.

The whole procedure for silanizing Au colloids takes about 3 hrs and can be performed at a particle concentration above ˜1 μM for 5 nm Au cores and above 0.1 μM for 10 nm cores. It was found that the same protocol works for both sizes of Au colloids.

There was no evidence of aggregation of particles during the silanization process. The plasmon peaks of citrate-Au solutions and silanized-Au solutions are at the same wavelength (˜524 nm vs ˜526 nm respectively). The UV-Vis spectrum of silanized Au solutions is stable for weeks, even though silanized Au solutions are stored at high concentrations in a 10 mM phosphate buffer. Gel electrophoresis mobility of silanized Au is qualitatively similar to that of silanized QD nanoparticles.

In various embodiments, the paramagnetic silica shell can act as a generic scaffold for multivalent contrast agents. The ability to grow silica shells around inorganic cores has several advantages: first the nanoparticles are extremely soluble in a wide variety of conditions (4<pH<11, and ionic strengths above 1M of phosphate buffer and 50 mM for buffers with divalent ions). Silanized nanoparticles are also stable in 1×PBS buffer at concentrations exceeding 50 μM. Although viscous at these concentrations, the solutions flow without resistance through capillaries used for the administration of the contrast agent in small animals. Second, the overall size of the nanoparticles remains small since the silica shell only adds a few nm to the particle diameter. It is estimated that the silica shell around the 5 nm Au cores is only 2 nm thick. This results in particle size of about 9 nm. Similarly, it is estimated that the silica shell adds about 2-4 nm to Au colloids of 10 nm in diameters, with a resulting total size of 15-18 nm. Finally, bioconjugation strategies to attach biomolecules to silica are well-developed. This is illustrated by the covalent linking of GdDOTA to the silanized nanoparticles. In certain embodiments, the thiols of the silica shells link together via linking agents, such as amine groups, on the paramagnetic chelated species using the ubiquitous sulfo-SMCC a linking protocol that follows closely one developed to covalently bind DNA to silanized QD as described by Gerion et al. (2002) J. American Chemical Society, 124(24): 7070-7074, and Gerion et al. 92002) Chemistry of Materials 14(5): 2113-2119.

The foregoing coating materials and methods are illustrative and not intended to be limiting. Using the teachings described herein, numerous other coating materials and methodologies will be available to one of skill in the art.

### III. Imaging Agents.

The nanoparticles described herein, can be attached to one or more imaging agents to provide a multimodal probe. In various embodiments the probes range in size from 1 to 100 nm, preferably from about 5 to about 50 nm, and more preferably from about 10 nm to about 30, 25, or 20 nm ad are highly soluble in high ionic strength buffers at pH ranging from ˜4 to 11. In various embodiments the imaging agent comprises an MRI imaging agent, a PET imaging agent, a NIR imaging agent, and ESR imaging agent, and the like.

MRI Imaging Agents.

In certain embodiments the imaging agent(s) comprise an MRI imaging agent attached to the nanoparticle. In various embodiments this provides, in effect, an outer paramagnetic or superparamagnetic shell (provided by an MRI imaging agent) attached to an inorganic core (the nanoparticle). One illustrative such probe can generally be described as a GdDOTA-SiO₂@Particle.

One great advantage of such a design is the possibility to select a material as the core particle that provides a signature different and distinguisnable from (e.g., orthogonal to) the one provided by the MRI agent (e.g., the paramagnetic GdDOTA-SiO₂ shell). For example, in certain embodiments, the core provides an optical component (fluorescence), while chelated paramagnetic or superparamagnetic ions linked to the outer shell contribute to MRI relaxivity. The strength of the design consists in the fact that the paramagnetic silica shell does not interfere with optical properties of the inorganic cores. For instance, the position of the plasmon peak of GdDOTA-SiO₂@Au shifts by less than 2 nm compared to citrate-stabilized Au. Similarly, the UV-Vis absorption and fluorescence emission of GdDOTA- SiO₂@QD are virtually similar to those of TOPO-capped QD. Although detailed investigations of fluorescence properties have not been performed in detail, side-to-side comparison of QD and GdDOTA-SiO₂@QD excited with a hand-held UV lamp shows that both solutions have fluorescence properties (color and intensity) undistinguishable to the naked eye. This approach, where optical and MRI properties arise from physically separated and weakly interacting entities, present several advantages over other approaches devised to make multivalent probes. For instance, attempts to dope the shell of fluorescent core/shell CdSe/ZnS nanoparticles with Mn impurities produce multimodal nanoparticles with quenched fluorescence and a relatively weak magnetic contribution.

In certain preferred embodiments, the multimodal probes are provided for use as magnetic resonance imaging (MRI) agents, where these multimodal probes exhibit increased relaxivities. In certain embodiments the probes possess spin-lattice (T₁) and spin-spin (T₂) relaxivities ranging from greater than about 200 mM⁻¹ s⁻¹, preferably greater than about 500 or 800 mM⁻¹ s⁻¹, and often ˜1,000 mM⁻¹ s⁻¹ for probes having ˜8 nm in diameter up to over 16,000 mM⁻¹ s⁻¹ probes of 15 nm diameter. In various embodiments the probes comprising an inorganic core of semiconductor or metallic nanoparticles covered with a a silica shell. In certain embodiments these silica-coated scaffolds are about 5-30 nm, preferably about 5-20 nm, more preferably about 10 nm in size and can be used to covalently anchor multiple imaging agents (e.g., GdDOTA molecules). In various embodiments typically the ratio of imaging agents:nanoparticle ranges from about 1:1, 10:1, 20:1, 30:1, 40:1, 45:1, 50:1, 75:1, 100:1, 200:1, 210:1. 220:1, 230:1, 240:1, 250:1, 260:1, 270:1, 280:1, 290:1, 300:1, 400:1, and 500:1.

Without being bound to a particular theory, it is believed that a multi-component 4mechanism contributes to these exceedingly high relaxivities. The mechanism involves a large number of GdDOTA moieties (or other MRI agents), the slowing of the tumbling rate of the MRI agents (e.g., GdDOTA), and the hydrophilicity of the silica surface . The number of MRI agents (e.g., GdDOTA) linked to the silica shell can be tuned, e.g., from ˜20 up to ˜250, whereby each unit contributes additively to the total relaxivity. Moreover, it was observed that the T₁ and T₂ relaxivities per GdDOTA unit is increased by a factor of ˜5 and ˜10 respectively when GdDOTA is bound to the silica shell compared to its mobile form in solution, and by a factor of ˜2 and ˜3 respectively compared to the case when GdDOTA is linked to the core nanoparticle through a flexible, weakly hydrophilic phospholipids layer.

It was further found that GdDOTA-SiO₂@QD and GdDOTA-SiO₂@Au with a diameter of about 8-10 nm (5 nm cores+2 nm silica shell) exhibit relaxivities in excess of r₁˜1000-2000 mM⁻¹s⁻¹and r₂˜3000 mM⁻¹s⁻¹ and are detectable at ˜100 nM concentrations. One obvious reason for this enhanced relaxivity is the number of GdDOTA molecules that decorate the silica surface. Chemical analysis indicates that about 45-50 GdDOTA are covering the silica surface of SiO₂@Au with 5 nm cores. More than 250-300 GdDOTA were measured around SiO₂@Au with 10 nm cores. As a result, relaxivities skyrocketed to ˜16,000 mM⁻¹s⁻¹ and the detection limit plunged in the 10 nM range.

In MRI experiments at clinical field strengths of 20 MHz and 60 MHz, the probes possess spin-lattice (T₁) and spin-spin (T₂) relaxivities ranging from ˜1,000 mM⁻¹ s⁻¹ for probes having ˜8 nm in diameter up to over 16,000 mM⁻¹ s⁻¹ for probes of 15 nm diameter. This represents a marked increase compared to iron oxide nanoparticles whose T₂ relaxivity levels off at 200-500 mM⁻¹ s⁻¹ for particle with a diameter over ˜50-100 nm, and drops to ˜18-30 mM⁻¹ s⁻¹ for very small superparamagnetic iron oxide particles (VSOP) of 7 nm in diameter. The increase in relaxivity of the silica-coated nanoprobes has been correlated to the number of paramagnetic ions covalently linked to the nanoparticles, ranging from ˜45 up to ˜260.

It was found that each GdDOTA contributes by about 23 mM⁻¹ s⁻¹ to the total T₁ and by about 54 mM⁻¹ s⁻¹ to the total T₂ relaxivity respectively. Transversal relaxivities are higher than longitudinal ones, despite the fact that the unbound chelated moieties affect mainly the longitudinal relaxation time. In contrast to superparamagnetic iron oxide particles where the water protons are kept at a distance of the magnetic core by a thick organic shell (i.e. dextran or polyethyleneglycol), the present design brings the paramagnetic moieties in direct contact with water protons, enhancing thus the contrast effect. In addition, the contrast power is modulated by the number of paramagnetic moieties linked to the silica shell and is only limited by the number of GdDOTA that can be packed on the surface. The sensitivity of the present probes is in the 100 nM range for particles of ˜8-10 nm and reaches 10 nM for particles having ˜15-18 nm in diameter. Theoretically, the relaxivities of the fluorescent probes are high enough to allow the detection of single cell by MRI.

Achieving high relaxivities does not require the use of an inorganic core of a specific nature, because the MRI contrast power is carried only by the paramagnetic silica shell. In it is believed to be possible to optimize the design and reach even higher relaxivity values by other combinations of lanthanide ions, chelators and inorganic nanoparticles. Any inorganic core that can be embedded into silica can be used as seed for high relaxivity contrast agents. This includes CdSe/ZnS, CdTe, Au, Ag, and any oxide nanocrystals, such as very small superparamagnetic iron oxide nanoparticles (for other examples, see also He et al. (2005) J. Phys. D: Appl. Phys., 38: 1342-1350).

The MRI imaging agents can include, but are not limited to positive contrast agents and/or negative contrast agents. Positive contrast agents cause a reduction in the T₁ relaxation time (increased signal intensity on T₁ weighted images). They (appearing bright on MRI) are typically small molecular weight compounds containing as their active element Gadolinium, Manganese, or Iron. All of these elements have unpaired electron spins in their outer shells and long relaxivities. A special group of negative contrast agents (appearing dark on MRI) include perfluorocarbons (perfluorochemicals), because their presence excludes the hydrogen atoms responsible for the signal in MR imaging.

Magnetic resonance imaging (MRI) is widely used clinically because it provides high spatial resolution images, particularly through the application of contrast agents which are currently employed in approximately 35% of all clinical MRI examinations. These are typically derived from iron particles or paramagnetic, predominantly Gd, complexes. One of the clinically approved, and commonly used contrast agents are Gd-DOTA (DOTA=1,4,7,10-tetrakis(carboxymethyl)-1,4,7,10-tetraazacyclododecane), which shows low toxicity and patient discomfort. Clinical safety results from its low osmolality, low viscosity, low chemotoxicity, high solubility, and high in vivo stability for the macrocylic complex.

The vast majority of MRI applications depend on the bulk biodistribution of the contrast agent rather than molecular targeting methods. As a small molecule, Gd agents get into the microvasculature around tumors, which is at a much higher density than normal tissue. This increased concentration of Gd in highly vascularized tissue around tumors is the basis for the MRI contrast mechanism. Thus, specifically targeted contrast agents, as described herein, are extremely useful for improving the ability of MRI to localize cancer.

In certain preferred embodiments, the MRI imaging or detection agent attached to the present multimodal probes are iron or paramagnetic radiotracers and/or complexes, including but not limited to gadolinium, xenon, iron oxide, copper, Gd³⁺-DOTA, and ⁶⁴Cu²⁺-DOTA.

Positron Emission Tomagraphy (PET) Imaging Agents.

The design of the multimodal probes can be easily extended to cover other imaging techniques such as, single photon emission computer tomography (SPECT), near infrared (NIR), electron spin resonance (ESR) imaging, and positron emission tomography (PET) imaging, and no difficulty is forseen to enhance the capability of the silica-coated probes by adding a targeting mechanism, e.g., as described herein.

In a PET scan, radioactive atoms are introduced into the body. The positrons emit when radionuclei decay, collide and annihilate with electrons in surrounding tissue, producing a pair of gamma ray photons moving in opposite directions, allowing gamma ray origin in the body be plotted and the density of the isotope in the body mapped by pair-detection events. A PET scan is especially useful in showing how tissue or an organ is functioning, as opposed to just showing structure.

The design of the probe allows its straightforward conversion into a PET probe. For example, this can be accomplished simply by attaching one or more PET imaging agents to the nanoparticle. A number of PET imaging radionuclides are known to those of skill in the art. These include, but are not limited to PET radiopharmaceuticals such as [¹¹C]choline, [¹⁸F]fluorodeoxyglucose (FDG), [¹¹C]methionine, [¹¹C]choline, [¹¹C]acetate, and [¹⁸F]fluorocholine as well as other radionuclides including but not limited to ¹¹C, ¹⁵O, ¹³N, ¹⁸F, ³⁵Cl, ⁷⁵Br, ⁸²Rb, ¹²⁴I, ⁶⁴Cu, ²²⁵Ac, ¹⁷⁷Lu, ¹¹¹In, ⁶⁶Ga, ⁶⁷Ga, ⁶⁸Ga, and the like.

Nuclear Magnetic Resonance (NMR) and Electron Spin Resonance Imaging Agents.

In certain embodiments the imaging agents comprise nuclear magnetic resonance (NMR) and/or electron spin resonance imaging agents. Such agents are well known to those of skill in the art and include, for example, nitroxides, and the like. In certain embodiments single-crystal ferrimagnetic spheres offer the advantages of high detectability through large magnetizations and narrow FMR lines. For example, yttrium-iron garnet Y₃Fe₅O₁₂ and γ-Fe₂O₃ are two well-known materials suitable for this application. Different dopants can be added to lower the spin resonance frequencies of these materials for medical applications. Magnetic garnets and spinels are also chemically inert and indestructible under normal environmental conditions. These examples are intended to be illustrative and not limiting.

Optical NIR-Based Tissue Imaging.

For in vivo optical imaging, the major challenge is that the dyes need to compete for light against the autofluorescing and light scattering nature of tissue, and the strong absorption profiles of biomolecules that absorb mostly in the visible region of the spectrum. The poor penetration of light through tissue limits the uses of these tags to subsurface locations, or requires specialized instrumentation such as a light probe. Theoretical calculations have proposed that NIR excitation light can penetrate tissue between 7-14 cm in depth with sensitive photon collection systems. In view of these observations, fluorophores have been developed that absorb in the NIR of the spectrum (650-900 nm).

Illustrative NIR dyes include a cyanine or indocyanine derivative. Such dyes include, but are not limited to Cy5.5, IRDye800, indocyanine green (ICG), indocyanine green derivatives and combinations thereof. In one specific embodiment, the dye includes a tetrasulfonic acid substituted indocyanine green (TS-ICG) (see, e.g., U.S. Pat. No. 6,913,743). Examples of suitable indocyanine include ICG and its derivatives. Such derivatives can include TS-ICG, TS-ICG carboxylic acid and TS-ICG dicarboxylic acid.

Additional examples include dyes available from Li-Cor, such as IR Dye 800CW.™., available from Li-Cor. Additional examples include dyes disclosed in U.S. Pat. No. 6,027,709. In one embodiment, the dye is N-(6-hydroxyhexyl)N′-(4-sulfonatobutyl)-3,3,3′,3′-tetramethylbenz(e)indo-dicarbocyanine, and/or N-(5-carboxypentyl)N′-(4-sulfonatobutyl)3,3,3′,3′-tetramethylbenz(e)indod-icarbocyanine.

These dyes have a maximum light absorption which occurs near 680 nm. They thus can be excited efficiently by commercially available laser diodes that are compact, reliable and inexpensive and emit light at this wavelength. Suitable commercially available lasers include, for example, Toshiba TOLD9225, TOLD9140 and TOLD9150, Phillips CQL806D, Blue Sky Research PS 015-00 and NEC NDL 32305U. This near infrared/far red wavelength also is advantageous in that the background fluorescence in this region normally is low in biological systems and high sensitivity can be achieved.

In certain embodiments the nanoparticles may be conjugated to a lissamine dye, such as lissamine rhodamine B sulfonyl chloride. Lissamine dyes are typically inexpensive dyes with attractive spectral properties. For example, examples have a molar extinction coefficient of 88,000 cm.sup.-1M.sup.-1 and good quantum efficient of about 95%. It absorbs at about 568 nm and emits at about 583 nm (in methanol) with a decent stokes shift and thus bright fluorescence.

In one embodiment, the detection and NIR imaging agent used in the multimodal probe is NIRQ820, a cyclohepta polymethine fluorochrome, ex/em=790/820 nm, a water soluble NIR fluorochrome with great chemical stability.

Additionally, there has been a recent increasing interest in the use of semi-conducting nanocrystals (Qdots) as a biological imaging tool. Conventional organic dyes are susceptible to photobleaching, while Qdots can be photostable with no observable fading for hours to days under biological imaging conditions. Qdots, have a much higher emission intensity and a longer lifetime, allowing for easy separation from background fluorescence. In addition, they have been shown to be stable under biological conditions for up to several months due to their high resistance to chemical and metabolic degradation. The photochemical properties of the nanoparticles are highlighted by a broad excitation spectrum which makes available a wide range of wavelengths that could be used to induce fluorescence, as well as a narrow emission spectrum that is largely red shifted from the absorption band, reducing backgrounds. The emissive and absorption wavelength of the nanoparticles are size dependent and are readily tunable. In addition, Qdots have been shown to be a very efficient two photon absorber, making it suitable for two photon spectroscopy and NIR excitation. This rapidly developing and minimally invasive technique has been demonstrated to effectively track events that occur deep in tissue.

Another benefit is the low cytotoxicity that has been demonstrated with the in vivo use of nanoparticles. The present silica-coated Qdots have no observable cytotoxicity at concentrations of 0.1 mg/mL (in vitro measurements, have shown that a minimum concentration of 1 nM is required for well resolved spectra, see, e.g., Larson et al. (2003) Science 300: 1434-1436; Hoshino et al. (2004) Biochem. Biophys. Res. Comm. 314: 46-53; and Zhang et al.(2006) Nano Lett. 6(4):800-808).

The photochemical properties of the nanoparticles are highlighted by a broad excitation spectrum which makes available a wide range of wavelengths that could be used to induce fluorescence, as well as a narrow emission spectrum that is largely red shifted from the absorption band, reducing backgrounds. The emissive and absorption wavelength of the nanoparticles are size dependent and are readily tunable. In addition, Qdot has been shown to be a very efficient two photon absorber(TPA)., making it suitable for two photon spectroscopy and NIR excitation. This involves the simultaneous excitation by two lower energy photons to reach the same excited energy state that can also be reached by a single higher energy photon. The cross sectional area is the two photon equivalent of the single photon absorption extinction coefficient, and Qdot has been shown to be an ideal multiphoton fluorophore because of its large cross-section. For CdSe Qdots (4.5 nm diameter), this was measured to be 47,000 Goeppert- Mayer units (GM) which is an order of magnitude higher than most organic dyes). Other than directly accessing the optical window, another advantage of TPA arises from the poor ability of most natural chromophores to efficiently absorb two photons. Since a small focal volume is required for TPA, scattering is negligible, and measurements can be made at different depths. This latter property can be used to generate highly resolved three dimensional fluorescent images that profile thick tissue samples. TPA fluorescence imaging has become a method of choice for tissue studies, and has been applied to neurophysiology, dermal physiology, and embryology as a non-invasive technique.

Moreover, as indicated above, quantum dots that are NIR emitters are well known to those of skill in the art. In certain embodiments the nanoparticle acts as the NIR emitter (detectable label), while in other emobidments, the attached imaging agent is a second nanoparticle (quantum dot).

### IV. Targeting Agents

Recent efforts have been directed at developing “molecular probes” that can locate and image diseased cells by engineering the imaging probe to recognize a marker of a disease state, on the cell surface. For example, it has been well established that the incorporation of a specificity element (such as an antibody) helps to selectively direct a tag to the desired cells.

The potential of these tags lies beyond diagnostics. For example, a real-time image of a tumor can also be used during surgery as a method to delineate the tumor margins. The molecular targeting mechanism also allows delivery of therapeutics to the tumor and imaging-guided intervention. Initially in one embodiment, PSMA is chosen as a molecular target, and a multimodal probe is made using a high quality internalizing scFv antibody as the targeting agent. Prostate-specific membrane antigen (PSMA) is present on prostate tumor cells and on cells that line the microvasculature of prostate tumors.

As an example, single chain antibodies against the target protein PMSA can be obtained. The antibodies are crosslinked to the linking agent, SMCC, a heterobifunctional crosslink that can react with a thiol group on the core nanoparticle and amine groups on the protein. The antibody-conjugated nanoparticle can be be tested for binding activity to PSMA, by BiaCore, and uptake by cultured prostate cancer cell lines.

More generally, in various embodiments, the probes described herein have attached thereto one or more targeting moities. In certain embodiments the targeting moieties are moities that specifically or preferentially bind to a particular (e.g., pre-selected target), e.g., a cancer marker.

Thus a targeting agent on the multimodal probe can comprise an affinity agent, e.g., an agent that specifically binds to a ligand. In general, any affinity agent useful in the prior art, in combination with a known in vivo ligand to provide specific recognition and detection of a disease state or diseased cell will find utility in the multimodal probes of the invention. Examples of suitable affinity agents, include but are not limited to, polysaccharides, lectins, selectins, nucleic acids (both monomeric and oligomeric), peptides, proteins, enzymes, lipids, monoclonal and polyclonal antibodies or fragments thereof (e.g., Fab, Fv, and scFv), and small molecules such as sugars, aptamers, drugs, and ligands.

In certain embodiments the targeting agent comprises a moiety (e.g., antibody, antibody fragment, single chain antibody, ligand, etc.) that specifically and/or preferentially binds a cancer marker.

A large number of cancer markers are known to those of skill in the art. Some cell surface components of cancer cells are common to normal cells and others are either qualitatively distinct for or quantitatively increased in tumor cells. Cell surface components common to both normal and malignant cells include, e.g., various kinds of receptors (e.g., certain hormone receptors), histocompatibility antigens, blood group antigens, and differentiation antigens. Receptors include, e.g., sheep erythrocyte receptor, hormone receptors, e.g., estrogen receptor and the like, transferrin receptor, Fc immunoglobulin receptor, nerve growth factor receptor, and the like. Blood group antigens include, e.g., the P determinant and M and N precursor (“T antigen”). Examples of differentiation antigens include surface immunoglobulin, and onco-neural antigens. Examples of histocompatibility antigens include HLA-A, HLA-B, HLA-DR (Ia-like). In cases where the cell-surface antigen is qualitatively distinct for cancer cells or quantitatively increased in cancer as compared to non-cancer tissues such cell surface markers will be useful as targets for localizing antibodies.

Antigens that are more restricted to tumor cells include, e.g., inappropriately (ectopically) expressed normal antigens, modified normal antigens, and neoantigens, such as embryonic and fetal antigens, viral antigens, and tumor-specific (or tumor-associated) antigens. Examples of embryonic and fetal antigens include, fetal onco-neural antigens, onco-fetal antigens, melanoma antigens, colorectal cancer antigens, lung cancer antigens, breast cancer antigens and the like. An example of a virus-associated antigen is the viral capsid antigen of Epstein-Barr virus.

Examples of tumor-specific or tumor-associated antigens include CEA, melanoma cell surface antigens, breast cancer cell surface antigens, lung cancer cell surface antigens, colorectal cancer cell surface antigens, gastric cancer cell surface antigens, pancreatic cancer cell surface antigens, glioma cell surface antigens, common sarcoma cell surface antigens, gastrointestinal cancer cell surface antigens, brain tumor cell surface antigens, esophageal cancer cell surface antigens, common epithelial cancer cell surface antigens, osteosarcoma cell surface antigens, fibrosarcoma cell surface antigens, urinary bladder cancer cell surface antigens, prostatic cancer cell surface antigens, renal cancer cell surface antigens, ovarian cancer cell surface antigens, testicular cancer cell surface antigens, endometrial cancer cell surface antigens, cervical cancer cell surface antigens, Hodgkin's disease cell surface antigens, lymphoma cell surface antigens, leukemic cell surface antigens, trophoblastic tumor cell surface antigens, and the like.

Tumor-specific antigens, by the strictest definition, are not present on normal cells during any stage of development or differentiation. These may result from mutation of structural genes, abnormal gene transcription or translation, abnormal post-translational modification of proteins, derepression of normally repressed genes, or insertion of genes from other cells or organisms (“transfection”). Since only about 1000 gene products have been identified for the approximately 1 million genes in mammalian cells, new tumor-associated antigens will probably be previously undefined normal gene products. An antigen need not be tumor-specific in the strictest sense to be useful as a target for localizing antibodies used for detection or therapy. For example, an inappropriate receptor may serve as a selective target for antibodies used for cancer detection or therapy.

In various embodiments the markers used in such methods include, but are not limited to MAGE-A3, GalNAcT, MART-1, PAX3, Mitf, TRP-2, and Tyrosinase. Methods for detecting metastatic breast, gastric, pancreas or colon cancer cells can utilize panels of markers such as C-Met, MAGE-A3, Stanniocalcin-1, mammoglobin, HSP27, Ga1NAcT, CK20, and β-HCG (see, e.g., U.S. Patent Publication 2004/0265845).

In certain embodiments a marker combination of tyrosinase and melanoma-associated antigens MART-1 and MAGE-A3 can be used to detect occult melanoma cells (see, e.g., Bostick et al. (1999) J. Clin. Oncol, 17: 3238-3244).

A wide variety of other cancer markers are known to those of skill in the art. Illustrative cancer markers include, for example, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Human mucins (e.g. MUC1) are known tumor markers as are gp100, tyrosinase, and MAGE, which are found in melanoma. Wild-type Wilms' tumor gene WT1 is expressed at high levels not only in most of acute myelocytic, acute lymphocytic, and chronic myelocytic leukemia, but also in various types of solid tumors including lung cancer.

Many kinds of tumor cells display unusual antigens that are either inappropriate for the cell type and/or its environment, or are only normally present during the organisms' development (e.g. fetal antigens). Examples of such antigens include the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies.

Other kinds of tumor cells display cell surface receptors that are rare or absent on the surfaces of healthy cells, and which are responsible for activating cellular signaling pathways that cause the unregulated growth and division of the tumor cell. Examples include (ErbB2). HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

An illustrative, but not limiting list of tumor markers is provided in Table 1. Antibodies to these and other cancer markers are known to those of skill in the art and can be obtained commercially or readily produces, e.g. using phage-display technology.

TABLE 1
Illustrative cancer markers and associated references, all of which are incorporated
herein by reference for the purpose of identifying the referenced tumor markers.

| Marker | Reference |
|---|---|
| 5 alpha reductase | Délos et al. (1998) Int J Cancer, 75: 6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (\grave{ }998) J Exp Med., 188(6): 1185-1190 |
| BAGE | Böel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al. (\grave{ }996) Blood, 87(11): 4770-4779 |
| CA-125 | Bast et al. (\grave{ }998) Int J Biol Markers, 13(4): 179-187 |
| CASP-8/FLICE | Mandruzzato et al. (1997) J Exp Med., 186(5): 785-793. |
| Cathepsins | Thomssen et al. (1995) Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 (791Tgp72) | Jarvis et al. (1997) Int J Cancer, 71(6): 1049-1055 |
| CDC27 | Wang et al. (1999) Science, 284(5418): 1351-1354 |
| CDK4 | Wölfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al. (1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunother., 44(4): 239-247 |
| gp-100-in4 | Kirkin et al. (1998) APMIS, 106(7): 665-679 |
| gp15 | Maeurer et al. (1996) Melanoma Res., 6(1): 11-24 |
| gp75/TRP-1 | Lewis et al. (1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al. (1991) Br J Cancer, 63(4): 534-540 |
| Hsp70 | Jaattela et al. (1998) EMBO J., 17(21): 6124-6134 |
| hTERT (telomerase) | Vonderheide et al. (1999) Immunity, 10: 673-679. 1999. |
| IGFR1 | Ellis et al. (1998) Breast Cancer Res. Treat., 52: 175-184 |
| IL-13R | Murata et al. (1997) Biochem Biophys Res Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Guéguen et al. (1998) J Immunol., 160(12): 6188-6194 |
| K-ras, H-ras, N-ras | Abrams et al. (1996) Semin Oncol., 23(1): 118-134 |
| KSA (CO17-1A) | Zhang et al. (1998) Clin Cancer Res., 4(2): 295-302 |
| LDLR-FUT | Caruso et al. (1998) Oncol Rep., 5(4): 927-930 |
| MAGE Family (MAGE1, | Marchand et al. (1999) Int J Cancer, 80(2): 219-230 |
| MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/MART-1 | Lewis and Houghton (1995) Semin Cancer Biol., 6(6): 321-327 |
| mesothelin | Chang et al. (1996) Proc. Natl. Acad. Sci., USA, 93(1): 136-140 |
| MIC A/B | Groh et al. (1998) Science, 279: 1737-1740 |
| MT-MMP's, such as MMP2, | Sato and Seiki (1996) J Biochem (Tokyo), 119(2): 209-215 |
| MMP3, MMP7, MMP9 | |
| Mox1 | Candia et al. (1992) Development, 116(4): 1123-1136 |
| Mucin, such as MUC-1, | Lewis and Houghton (1995) Semin Cancer Biol., 6(6): 321-327 |
| MUC-2, MUC-3, and MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grøndahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) Biochem Biophys Res Commun., 130(1): 293-300 |
| Progenipoietin | — |
| PSA | Sanda et al. (1999) Urology, 53(2): 260-266. |
| PSM | Kawakami et al. (1997) Cancer Res., 57(12): 2321-2324 |
| RAGE-1 | Gaugler et al. (1996) Immunogenetics, 44(5): 323-330 |
| Rb | Dosaka-Akita et al. (1997) Cancer, 79(7): 1329-1337 |
| RCAS1 | Sonoda et al. (1996) Cancer, 77(8): 1501-1509. |
| SART-1 | Kikuchi et al. (1999(Int J Cancer, 81(3): 459-466 |
| SSX gene family | Gure et al. (1997) Int J Cancer, 72(6): 965-971 |
| STAT3 | Bromberg et al. (1999) Cell, 98(3): 295-303 |
| STn (mucin assoc.) | Sandmaier et al. (1999) J Immunother., 22(1): 54-66 |
| TAG-72 | Kuroki et al. (1990)Cancer Res., 50(16): 4872-4879 |
| TGF-α | Imanishi et al. (1989) Br J Cancer, 59(5): 761-765 |
| TGF-β | Picon et al. (1998) Cancer Epidemiol Biomarkers Prey, 7(6): 497-504 |
| Thymosin β 15 | Bao et al. (1996) Nature Medicine. 2(12), 1322-1328 |
| IFN-α | Moradi et al. (1993) Cancer, 72(8): 2433-2440 |
| TPA | Maulard et al. (1994) Cancer, 73(2): 394-398 |
| TPI | Nishida et al. (1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |
| S-transferase | |

The foregoing examples are meant to be illustrative and not limiting. One or skill will recognize that these and/or other cancer markers.

In certain embodiments, the targeting agent is a signal peptide, as described in co-pending International Patent Application PCT/US2005/031386. Any suitable signal peptide can be used in the signal peptide-nanoparticle conjugates of the invention. The peptide should be able to target (i.e., mediate entry and accumulation) of a signal peptide-nanoparticle to a subcellular compartment and/or organelle of interest. Signal peptides are typically about about 5 to about 200, about 10 to about 150, about 15 to about 100, or about 20 to about 50 amino acids in length. Suitable signal peptides include, e.g., nuclear localization signal peptides, peroxisome-targeting signal peptides, cell membrane-targeting signal peptides, mitochondrial-targeting signal peptides, and endoplasmic reticulum-targeting signal peptides, and trans-Golgi body-targeting signal peptides. Signal peptides may also target the signal peptide-nanoparticle conjugates to any cell surface receptor including e.g. epidermal growth factor receptors (EGFR), fibroblast growth factor receptors (FGFR), vascular endothelial cell growth factor receptor (VEGFR), integrins, chemokine receptors, platelet-derived growth factor receptor (PDGFR), tumor growth factor receptora, and tumor necrosis factor receptors (TNF).

Nuclear localization signal peptides typically comprise positively charged amino acids. Endoplasmic reticulum targeting signal peptides typically comprise about 5 to about 10 hydrophobic amino acids. Mitochondria targeting signal peptides are typically about 5 to about 10 amino acids in length and comprise a combination of hydrophobic amino acids and postively charged amino acids. Peroxisome targeting signal peptides include PTS1, a 3 amino acid peptide and PTS2, a 26-36 amino acid peptide.

Examples of signal peptide sequences include but are not limited to the the sequences shown in Table 1.

TABLE 1
Illustrative signal peptide sequences.:

| Target | Source | Sequence |
|---|---|---|
| Nucleus | SV-40 | PPKKKRKVPPKKKRKV |
| | large T | (SEQ ID NO: 1) |
| | antigen | |
| Nucleus | Tat | YGRKKRRQRRR |
| | protein | (SEQ ID NO: 2) |
| | of HIV | |
| Endoplasmic | | KDELA KDELA KDELA KDEL |
| Reticulum | | (SEQ ID NO: 3) |
| Mito- | Cyto- | SVTTPLLLRGLTGSARRLPVPRAKIHSL |
| chondria | chrome C | (SEQ ID NO: 4) |
| | oxidase | |
| Peroxisome | | SKLA SKLA SKLA SKLA |
| | | (SEQ ID NO: 5) |
| Cell | | KLNPPDESGPCMSCKCVLS |
| Membrane | | (SEQ ID NO: 6) |
| Cell | GAP-43 | MLCCMRRTKQVEKNDEDQKI |
| Membrane | | (SEQ ID NO: 7) |

In one illustrative embodiment, to target the present multi-modal probe to breast cancer, the nanoparticle is attached to a single-chain antibody against ErbB2, which is a protein in the EGFR family overexpressed in 15% to >50% of breast cancers, depending on the stage of the disease. The nanoparticle is highly fluorescent with a high quantum yield, and the clustering of the Gd chelating compound or zero-field MRI agent is demonstrated to be at least 500 per nanoparticle. In addition, these nanoparticles can reflect the changes in microenvironment around them, so that tumor status can be closely followed in real time during therapy.

In another embodiment, the targeting agent is a peptide containing the sequence of HS.SKLQ-LAAAC (SEQ ID NO:8) which has been shown to have very high specificity for proteolytically active PSA (see, e.g., Denmeade et al. (1997) Cancer Res 57, 4924-4930).

A number of cancer marker specific antibodies are described, for example, in U.S. Pat. Nos. 7,335,744, 7,332,585, 7,332,580, 7,312,044, 5,977,322, and the like.

The foregoing targeting agents are illustrative and not intended to be limiting. Other suitable targeting agents will be recognized by one of skill in the art.

### V. Therapeutic Agents

In certain embodiments, the multimodal probes of the present invention can further comprise any therapeutic agent that can be conjugated to the nanoparticle including, but not limited to, nucleic acids (both monomeric and oligomeric), proteins, enzymes, lipids, antibodies, polysaccharides, lectins, selectins, and small molecules such as sugars, peptides, aptamers, drugs, and ligands. The therapeutic agent can be conjugated or encapsulated in any type of delivery molecule or carrier such as microgels, liposomes, or lipids. In one embodiment, the therapeutic agent is directed to cancer such as breast or prostate cancer.

Photodynamic Therapeutic Agents.

In another embodiment, in addition to imaging and conjugation with MRI/PET modality, we will also exploit the photodynamic property of Qdots for therapy. Photodynamic therapy (PDT) is an emerging cancer treatment that takes advantage of the interaction between light and a photosensitizing agent to initiate apoptosis of cancer cells as also described in Samia et al. (2003) J Am Chem Soc 125: 15736-15737 (2003). In PDT, the photosensitizing agent becomes activated by light but does not react directly with cells and tissues. Instead, it transfers its triplet state energy to nearby oxygen molecules to form reactive singlet oxygen (¹O₂) species, which cause cytotoxic reactions in the cells (see, e.g., Sharmanet al. (2000) Methods Enzymol 319: 376-400. The increasing popularity of this treatment method is largely due to its selectivity: only tissues that are simultaneously exposed to the photosensitizer and light, in the presence of oxygen, are the ones subjected to the cytotoxic reactions during PDT. A first-generation photosensitizer that has been accepted for clinical use is the hematophorphyrin derivative, Photofrin (see, e.g., .Dougherty (1998) Crit Rev Oncol Hematol 2: 83-116). Among the more promising second-generation photosensitizers that are currently being evaluated for PDT applications are the phthalocyanines (Pc's). Pc derivatives have stronger absorbance at long wavelengths and chemical tunability through substituent addition on the periphery of the macrocycle or on the axial ligands. Quantum dots offers great promise in PDT applications. They can be tuned to emit into the abosorption region of the phthalocyanine photosensitizers. Furthermore, due to their large transition dipole moment, QDs are strong absorbers, making them ideal agents for PDT applications. More importantly, the surface coating of QDs can be functionalized to be linked to Pc4.

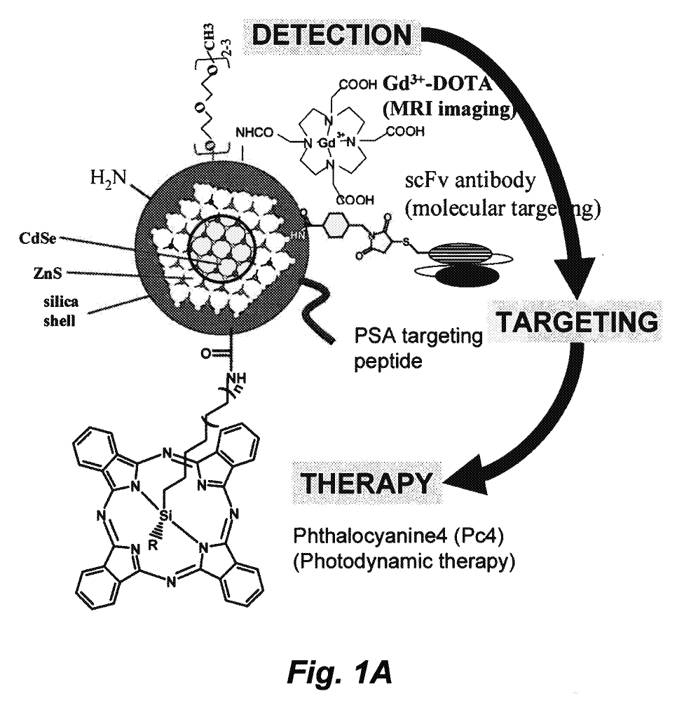

Thus, in a one illustrative embodiment, as shown in FIG. 1A, the semiconductor nanoparticle (nanocrystal) is comprised of a CdSe core with a silica shell, having amino and PEG groups displayed, and further comprising a Ge³⁺-DOTA attached to the nanoparticle via a linker, an scFv antibody and/or a targeting peptide, and a therapeutic for photodynamic therapy.

In one illustrative embodiment that targets prostate cancer, the scFv antibody is MEMD, the targeting peptide is HSSKLQ-LAAAC (SEQ ID NO: 8), and the therapeutic for photodynamic therapy is Phthalocyanine4 (Pc4). In another embodiment, for breast cancer, the scFv antibody acts as the therapeutic agent such as an anti-ErbB2 (e.g., C6.5 and C6ML3-9 antibodies) or an anti-Her2 antibody.

ESR Heating Therapeutic Agents.

In another embodiment, the therapeutic moiety and/or the nanoparticle comprises a moiety suitable for electron spin resonance heating. Electron spin resonance can be used for effective and local heating of superparamagnetic particles, preferably superparamagnetic nanoparticles in, or adjacent to, biological specimens (e.g., cells, tissues, organs, organisms, etc.). The local heating obtainable is effective in the hyperthermic (e.g., thermal ablation, temperature-induced apoptosis, etc.) treatment of cancers (or other conditions characterized by cellular hyperproliferation), the cosmetic ablation of tissues, and the like. Methods and reagents for ESR heating are described in U.S. Patent Publications 2006/0269612 and 2005/0118102

Anti-Cancer Pharmaceuticals.

In other embodiments, the therapeutic moiety comprises an anti-cancer pharmaceutical. One useful class of anti-cancer pharmaceutical includes the retinoids. Retinoids are useful in treating a wide variety of epithelial cell carcinomas, including, but not limited to pulmonary, head, neck, esophagus, adrenal, prostate, ovary, testes, pancreas, and gut.

Retinoic acid, analogues, derivatives, and mimetics are well known to those of skill in the art. Such retinoids include, but are not limited to retinoic acid, ceramide-generating retinoid such as fenretinide (see, e.g., U.S. Pat. No. 6,352,844), 13-cis retinoic acid (see, e.g., U.S. Pat. Nos. 6,794,416, 6,339,107, 6,177,579. 6,124,485, etc.), 9-cis retinoic acid (see, e.g., U.S. Pat. Nos. 5,932,622, 5,929,057, etc.), 9-cis retinoic acid esters and amides (see, e.g., U.S. Pat. No. 5,837,728), 11-cis retinoic acid (see, e.g., U.S. Pat. No. 5,719,195), all trans retinoic acid (see, e.g., U.S. Pat. Nos. 4,885,311, 4,994,491, 5,124,356, etc.), 9-(Z)-retinoic acid (see, e.g., U.S. Pat. Nos. 5,504,230, 5,424,465, etc.), retinoic acid mimetic anlides (see, e.g., U.S. Pat. No. 6,319,939), ethynylheteroaromatic-acids having retinoic acid-like activity (see, e.g., U.S. Pat. Nos. 4,980,484, 4,927,947, 4,923,884 Ethynylheteroaromatic-acids having retinoic acid-like activity, U.S. Pat. No. 4,739,098, etc.) aromatic retinoic acid analogues (see, e.g., U.S. Pat. No. 4,532,343), N-heterocyclic retinoic acid analogues (see, e.g., U.S. Pat. No. 4,526,7874), naphtenic and heterocyclic retinoic acid analogues (see, e.g., U.S. Pat. No. 518,609), open chain analogues of retinoic acid (see, e.g., U.S. Pat. No. 4,490,414), entaerythritol and monobenzal acetals of retinoic acid esters (see, e.g., U.S. Pat. No. 4,464,389), naphthenic and heterocyclic retinoic acid analogues (see, e.g., U.S. Pat. No. 4,456,618), azetidinone derivatives of retinoic acid (see, e.g., U.S. Pat. No. 4,456,618), and the like.

In various embodiments the retinoic acid, retinoic acid analogue, derivative, or mimetics can be coupled (e.g., conjugated) to the nanoparticle or it can be contained within a liposome or complexed with a lipid or a polymeric nanoparticle that is coupled to the nanoparticle e.g. as described herein.

In certain embodiments the methods and compositions of this invention can be used to deliver other cancer therapeutics instead of or in addition to the retinoic acid or retinoic acid analogue/derivative. Such agents include, but are not limited to alkylating agents (e.g., mechlorethamine (Mustargen), cyclophosphamide (Cytoxan, Neosar), ifosfamide (Ifex), phenylalanine mustard; melphalen (Alkeran), chlorambucol (Leukeran), uracil mustard, estramustine (Emcyt), thiotepa (Thioplex), busulfan (Myerlan), lomustine (CeeNU), carmustine (BiCNU, BCNU), streptozocin (Zanosar), dacarbazine (DTIC-Dome), cis-platinum, cisplatin (Platinol, Platinol AQ), carboplatin (Paraplatin), altretamine (Hexalen), etc.), antimetabolites (e.g. methotrexate (Amethopterin, Folex, Mexate, Rheumatrex), 5-fluoruracil (Adrucil, Efudex, Fluoroplex), floxuridine, 5-fluorodeoxyuridine (FUDR), capecitabine (Xeloda), fludarabine: (Fludara), cytosine arabinoside (Cytaribine, Cytosar, ARA-C), 6-mercaptopurine (Purinethol), 6-thioguanine (Thioguanine), gemcitabine (Gemzar), cladribine (Leustatin), deoxycoformycin; pentostatin (Nipent), etc.), antibiotics (e.g. doxorubicin (Adriamycin, Rubex, Doxil, Daunoxome-liposomal preparation), daunorubicin (Daunomycin, Cerubidine), idarubicin (Idamycin), valrubicin (Valstar), mitoxantrone (Novantrone), dactinomycin (Actinomycin D, Cosmegen), mithramycin, plicamycin (Mithracin), mitomycin C (Mutamycin), bleomycin (Blenoxane), procarbazine (Matulane), etc.), mitotic inhibitors (e.g. paclitaxel (Taxol), docetaxel (Taxotere), vinblatine sulfate (Velban, Velsar, VLB), vincristine sulfate (Oncovin, Vincasar PFS, Vincrex), vinorelbine sulfate (Navelbine), etc.), chromatin function inhibitors (e.g., topotecan (Camptosar), irinotecan (Hycamtin), etoposide (VP-16, VePesid, Toposar), teniposide (VM-26, Vumon), etc.), hormones and hormone inhibitors (e.g. diethylstilbesterol (Stilbesterol, Stilphostrol), estradiol, estrogen, esterified estrogens (Estratab, Menest), estramustine (Emcyt), tamoxifen (Nolvadex), toremifene (Fareston) anastrozole (Arimidex), letrozole (Femara), 17-OH-progesterone, medroxyprogesterone, megestrol acetate (Megace), goserelin (Zoladex), leuprolide (Leupron), testosteraone, methyltestosterone, fluoxmesterone (Android-F, Halotestin), flutamide (Eulexin), bicalutamide (Casodex), nilutamide (Nilandron), etc.) INHIBITORS OF SYNTHESIS (e.g., aminoglutethimide (Cytadren), ketoconazole (Nizoral), etc.), immunomodulators (e.g., rituximab (Rituxan), trastuzumab (Herceptin), denileukin diftitox (Ontak), levamisole (Ergamisol), bacillus Calmette-Guerin, BCG (TheraCys, TICE BCG), interferon alpha-2a, alpha 2b (Roferon-A, Intron A), interleukin-2, aldesleukin (ProLeukin), etc.) and other agents such as 1-aspariginase (Elspar, Kidrolase), pegaspasgase (Oncaspar), hydroxyurea (Hydrea, Doxia), leucovorin (Wellcovorin), mitotane (Lysodren), porfimer (Photofrin), tretinoin (Veasnoid), and the like.

Ribozymes.

In certain embodiments the therapeutic agents include ribozymes (see, e.g., Scanlon (2004) Curr Pharm Biotechnol., 5: 415-420; Citti and Rainaldi (2005) Curr Gene Ther., 5: 11-24). The ribozymes are typically provided encapsulated in a liposome or nanocapsule or admixed in a lipid. In addition to possessing catalytic activities as well as binding capacity to the RNA, the hammerhead ribozymes can cause RNase-dependent degradation of the target double-stranded RNA (dsRNA). Ribozymes can be directed against a number of different targets in the treatment of a cancer. Thus for example, a modified chimeric ribozyme targeting VEGF receptor, flt-1 (Angiozyme), was developed by Ribozyme Inc., which is now renamed Sirna Therapeutics Inc. (Boulder, Colo.).

Antisense and/or Antigene Molecules.

In certain embodiments the therapeutic agents include antisense and/or antigene molecules. Antigene oligonucleotides are antisense sequences that can insert themselves into a section of a DNA to form a triple helix, and thus inhibit transcription. Recognition of a duplex sequence by a third strand of DNA or RNA via the major groove is the basis of the formation of a triple helix. Typically, stable triplexes form on polypurine:polypyrimidine tracts. The third strand, depending on the target sequence, may consist of purines or pyrimidines, and the complex is stabilized by two Hoogsteen hydrogen bonds between third strand bases and the bases in the purine strand of the duplex. Triple helix is an inherent property of DNA and requires no additional enzymes or proteins.

Peptide nucleic acids (PNAs) are DNA analogs consisting of nucleobases attached to a peptide backbone of N-(2-aminoethyl)glycine residues. The phosphate charges are replaced with neutral peptide linkage, resulting in a stable hybrid between PNA and DNA or RNA strands. In addition, they can form triplexes by Hoogsteen pairing on polypurine and polypyrimidine targets. PNAs are resistant to degradation, form stable complexes on DNA targets and show high sequence selectivity, making them very attractive for cancer therapy (see, e.g., Dean (2000) Adv Drug Deliv Rev, 44: 81-95; Nielsen (2001) Curr Med Chem 8: 545-550; Braasch and Corey (2002) Biochemistry 41: 4503-4510; and the like.).

Antisense oligonucleotides are the most widely used unmodified or chemically modified single-stranded RNA or DNA molecules. One of the first reports to show in vivo activity was of a phosphodiester oligonucleotide directed against N-MYC that caused a decrease in tumor mass associated with loss of N-MYC protein in a subcutaneously transplanted neuroepithelioma in mice (Whitesell et al. (1991) Antisense Res Dev 1: 343-350). As the phosphodiester bond is highly susceptible to degradation, the development of phosphorothioate chemistry, which contains a sulfur atom in each internucleotide linkage instead of oxygen, revolutionized this field because of its stability (Lebedeva et al. (2001) Annu Rev Pharmacol Toxicol 41: 403-419; Crooke (2004) Annu Rev Med 55: 61-95; and the like).

The phosphorothioate antisense has shown the broadest range of activity in preclinical and clinical studies (ISIS Pharmaceuticals Inc., Carlsbad, Calif.; Genta Inc., Berkeley Heights, N.J.; Hybridon Inc., Cambridge, Mass.).

Certain second-generation antisense oligonucleotides comprise alkyl modifications at the 2′ position of the ribose and the development of novel chemically modified nucleotides with improved properties such as enhanced serum stability, higher target affinity and low toxicity (Kurreck (2003) Eur J Biochem 270: 1628-1644). One such modification in oligomer chemistry has led to the development of the phosphorodiamidate morpholino oligomers (PMO) by AVI BioPharma Inc. (Portland, Oreg.), which are non-ionic antisense agents that inhibit gene expression by binding to RNA and sterically blocking processing or translation in an RNaseH-independent manner. PMO antisense agents have revealed excellent safety profile and efficacy in multiple disease models including cancer preclinical studies targeting for example, c-myc, and/or MMP-9 (see, e.g., Hudziak et al. (2000) Antisense Nucleic Acid Drug Dev 10: 163-176; Devi et al. (2002) Prostate 53: 200-210; Knapp et al. (2003) Anticancer Drugs 14: 39-47; London et al (2003) Cancer Gene Ther 10: 823-832; Devi (2002) Curr Opin Mol Ther 4: 138-148; Ko et al. (2004) J Urol. 172: 1140-1144; Iversen et al. (2003) Clin Cancer Res 9: 2510-2519; and the like).

RNAi.

In certain embodiments the nanoparticle probes of this invention can be used to deliver an siRNA. Preclinical cancer studies have shown inhibition of growth and survival of tumor cells by RNAi-mediated downregulation of several key oncogenes or tumor-promoting genes, including growth and angiogenic factors or their receptors (vascular endothelial growth factor, epidermal growth factor receptor), human telomerase (hTR, hTERT), viral oncogenes (papillomavirus E6 and E7) or translocated oncogenes (BCR-abl). Various studies are reporting in vivo activity and the potential of RNAi to suppress tumor growth. These include an intratumoral injection of an shRNA-adenoviral vector construct targeting a cell cycle regulator causing inhibition of subcutaneous small cell lung tumor in mice, and systemic administration of an siRNA targeting a carcinoembryonic antigen-related cell adhesion molecule (CEACAM6) in mice with subcutaneously xenografted pancreatic adenocarcinoma cells. In another report, direct injection of a plasmid vector expressing shRNAs to matrix metalloproteinase MMP-9 and a cathepsin showed efficacy in established glioblastoma.

Illustrative targets for siRNA as a cancer therapeutic include, but are not limited to Bax or Bcl-2 targeting the apoptosis pathway (see, e.g., Grzmil et al. (2003) Am J Pathol., 163: 543-552; Yin et al. (2003) J Exp Ther Oncol., 3: 194-204), focal adhesion kinase (FAK targeting angiogenesis) (see, e.g., Duxbury (2003) Biochem Biophys Res Commun., 311: 786-792)' adhesion matrix metalloproteinase (Sanceau (2003) J Biol Chem 278: 36537-36546), VEGF (see, e.g., Yin et al. (2003) J Exp Ther Oncol. 3: 194-204; Zhang (2003) Biochem Biophys Res Commun., 303: 1169-1178)' fatty acid synthase (De Schrijver et al. (2003) Cancer Res., 63: 3799-3804.), MDR (Nieth et al. (2003) FEBS Lett., 545: 144-150)' H-Ras (Yin et al. (2003) J Exp Ther Oncol. 3: 194-204; Zhang (2003) Biochem Biophys Res Commun., 303: 1169-1178), K-Ras (Lois et al. (2001) Curr Opin Immunol., 13: 496-504), PLK-1 (Spankuch-Schmitt et al. (2002) J Natl Cancer Inst., 94: 1863-1877), TGF-β (Yin et al. (2003) J Exp Ther Oncol. 3: 194-204)' STAT3 (Konnikova et al. (2003) BMC Cancer3: 23)' EGFR (Nagy et al. (2003) Exp Cell Res., 285: 39-49; Zhang et al. (2004) Acta Pharmacol., 25: 61-67), PKC-α (Yin et al. (2003) J Exp Ther Oncol. 3: 194-204) Epstein-Barr virus (Li et al. (2004) Biochem Biophys Res Commun., 315: 212-218) HPV E6 (Butzet al. (2003) Oncogene 22: 5938-5945), BCR-Abl (Wohlbold et al. (2003) Blood 102: 2236-2239; Fuchs et al. (2002) Oncogene, 21: 5716-5724), telomerase (Kosciolek et al. (2003) Mol Cancer Ther. 2: 209-216), and the like.

### VI. Linking Agents

The imaging, and/or targeting, and/or therapeutic agents are typically attached to the nanoparticles via a linking agent. The agents and nanoparticle can be conjugated via a single linking agent or multiple linking agents. For example, the imaging agent and nanoparticle may be conjugated via a single multifunctional (e.g., bi-, tri-, or tetra-) linking agent or a pair of complementary linking agents. In another embodiment, the targeting agent and the nanoparticle are conjugated via two, three, or more linking agents. Suitable linking agents include, but are not limited to, e.g., functional groups, affinity agents, stabilizing groups, and combinations thereof.

In certain embodiments the linking agent is or comprises a functional group. Functional groups include monofunctional linkers comprising a reactive group as well as multifunctional crosslinkers comprising two or more reactive groups capable of forming a bond with two or more different functional targets (e.g., labels, proteins, macromolecules, semiconductor nanocrystals, or substrate). In some preferred embodiments, the multifunctional crosslinkers are heterobifunctional crosslinkers comprising two or more different reactive groups.

Suitable reactive groups include, but are not limited to thiol (—SH), carboxylate (COOH), carboxyl (—COOH), carbonyl, amine (NH₂), hydroxyl (—OH), aldehyde (—CHO), alcohol (ROH), ketone (R₂CO), active hydrogen, ester, sulfhydryl (SH), phosphate (—PO₃), or photoreactive moieties. Amine reactive groups include, but are not limited to e.g., isothiocyanates, isocyanates, acyl azides, NHS esters, sulfonyl chlorides, aldehydes and glyoxals, epoxides and oxiranes, carbonates, arylating agents, imidoesters, carbodiimides, and anhydrides. Thiol-reactive groups include, but are not limited to e.g., haloacetyl and alkyl halide derivates, maleimides, aziridines, acryloyl derivatives, arylating agents, and thiol-disulfides exchange reagents. Carboxylate reactive groups include, but are not limited to e.g., diazoalkanes and diazoacetyl compounds, such as carbonyldiimidazoles and carbodiimides. Hydroxyl reactive groups include, but are not limited to e.g., epoxides and oxiranes, carbonyldiimidazole, oxidation with periodate, N,N′-disuccinimidyl carbonate or N-hydroxylsuccimidyl chloroformate, enzymatic oxidation, alkyl halogens, and isocyanates. Aldehyde and ketone reactive groups include, but are not limited to e.g., hydrazine derivatives for schiff base formation or reduction amination. Active hydrogen reactive groups include, but are not limited to e.g., diazonium derivatives for mannich condensation and iodination reactions. Photoreactive groups include, but are not limited to e.g., aryl azides and halogenated aryl azides, benzophenones, diazo compounds, and diazirine derivatives.

Other suitable reactive groups and classes of reactions useful in practicing the present invention include those that are well known in the art of bioconjugate chemistry. Currently favored classes of reactions available with reactive chelates are those which proceed under relatively mild conditions. These include, but are not limited to, nucleophilic substitutions (e.g., reactions of amines and alcohols with acyl halides, active esters), electrophilic substitutions (e.g., enamine reactions), and additions to carbon-carbon and carbon-heteroatom multiple bonds (e.g., Michael reaction, Diels-Alder addition). These and other useful reactions are discussed in, for example, March (1985) Advanced Organic Chemistry, 3rd Ed., John Wiley & Sons, New York, Hermanson (1996) Bioconjugate Techniques, Academic Press, San Diego; and Feeney et al. (1982) Modification of Proteins; Advances in Chemistry Series, Vol. 198, American Chemical Society, Washington, D.C..

In one preferred embodiment, the linking agent is a chelator. For example, in a preferred embodiment, the chelator comprising the molecule, DOTA (DOTA=1,4,7,10-tetrakis(carboxymethyl)-1,4,7,10-tetraazacyclododecane), that can readily be labeled with a radiolabel, such as Gd³⁺ and ⁶⁴Cu, resulting in Gd³⁺-DOTA and ⁶⁴Cu-DOTA respectively, attached to the quantum dot (nanoparticle). Optical properties of the cores (fluorescence emission or plasmon position) are not affected by the addition of a silica shell or the presence of chelated paramagnetic ions. Other suitable chelates are known to those of skill in the art, for example, 1,4,7-triazacyclononane-N,N′,N″-triacetic acid (NOTA) derivatives being among the most well known (see, e.g., Lee et al. (1997) Nucl Med Biol. 24:225-23019).

In certain embodiments the linking agent is a heterobifunctional crosslinker comprising two different reactive groups that form a heterocyclic ring that can interact with a peptide. For example, a heterobifunctional crosslinker such as cysteine may comprise an amine reactive group and a thiol-reactive group can interact with an aldehyde on a derivatized peptide. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. In one embodiment, the heterobifunctional crosslinker is SMCC.

In some embodiments, an affinity agent (e.g., agents that specifically binds to a ligand) is the linking agent. In these embodiments, a first linking agent is bound to the semiconductor nanocrystal (nanoparticle) and a second linking agent is bound to the imaging, targeting or therapeutic agent. Affinity agents include receptor-ligand pairs, antibody-antigen pairs and other binding partners such as streptavidin/avidin and biotin. In one illustrative embodiment, the first linking agent is streptavidin or avidin and the second linking agent is biotin. the streptavidin or avidin is bound to the nanoparticle and a biotinylated agent (e.g., biotinylated imaging agent, biotinylated therapeutic, biotinylated antibody, etc.) is conjugated to the nanoparticle via streptavidin/avidin-biotin linkage. In some embodiments, other biotinylated radiolabel, peptides, proteins, antibodies, dyes, probes and other small molecules are attached to the streptavidin or avidin, and thus the nanoparticle.

Methods of producing chelates suitable for coupling to various targeting moieties are well known to those of skill in the art (see, e.g., U.S. Pat. Nos. 6,190,923, 6,187,285, 6,183,721, 6,177,562, 6,159,445, 6,153,775, 6,149,890, 6,143,276, 6,143,274, 6,139,819, 6,132,764, 6,123,923, 6,123,921, 6,120,768, 6,120,751, 6,117,412, 6,106,866, 6,096,290, 6,093,382, 6,090,800, 6,090,408, 6,088,613, 6,077,499, 6,075,010, 6,071,494, 6,071,490, 6,060,040, 6,056,939, 6,051,207, 6,048,979, 6,045,821, 6,045,775, 6,030,840, 6,028,066, 6,022,966, 6,022,523, 6,022,522, 6,017,522, 6,015,897, 6,010,682, 6,010,681, 6,004,533, and 6,001,329).

VII. Detection and/or Treatment of Diseased Cells and/or Tissues.

In one illustrative embodiment, the the multimodal probe, (1) detects cells by MRI, PET, ESR, SPECT, and/or deep tissue Near Infrared (NIR) imaging, and is capable of detecting diseased cells with greater sensitivity than is possible with existing technologies, (2) targets molecules that localize on the surface of diseased cells, and (3) initiates apoptosis of diseased cells by specific targeting of a therapeutic modality (e.g., anti-cancer pharmaceutical, local infrared lasermediated photodynamic therapy (PDT), etc.).

In one embodiment, this invention provides a nanoparticle-based technology platform for multimodal cancer imaging and therapy that, (1) detects cancer by MRI, PET or deep tissue Near Infrared (NIR) imaging, and is capable of detecting cancer cells with greater sensitivity than is possible with existing technologies, (2) targets molecules that localize on the surface of cancer cells, and (3) initiates apoptosis of cancer cells by local infrared laser-mediated photodynamic therapy (PDT).

In certain embodiments the platform comprises a nanoparticle probe comprising a silanized semiconductor nanocrystal having a agent for detection and/or imaging, a targeting agent, and a therapeutic agent attached thereto.

The present multimodal probes may be used in cancer detection and treatment, and are contemplated for use in cancers such as prostate, breast, brain, epithelial, and the like. The probes can be used to image and/or specifically or preferentially deliver a therapeutic to the cancer site.

Primary tumors less than about 2 mm in diameter are below the detection limit of most clinical imaging techniques. The clinical impact of inability to detect small tumors is that cancers are frequently not detected until the have progressed to a stage when the probability of curative treatment is significantly reduced. Rapid, sensitive, specific and noninvasive imaging techniques are urgently needed to detect cancer in its earliest stages to improve clinical outcomes. In addition, targeted, molecular image-guided intervention has not been previously evaluated adequately in prostate cancer to allow highly localized, non-invasive therapeutic treatments.

In various embodiments the nanoparticle probes described herein are administered to a subject (e.g., a human or a non-human mammal) to act as an imaging reagent (e.g., a contrast agent) and/or to provide a therapeutic benefit (e.g., so specifically and/or preferentially deliver a therapeutic moiety to a target cell or tissue). In certain embodiments the nanoparticle probes are systemically administered to a subject in need thereof. In certain embodiments they nanoparticle probes are delivered locally to a disease (e.g., tumor) site. In certain embodiments the nanoparticle probes are used to image a site during an operative procedure and/or to therapeutically treat that same site during or after a surgical procedure.

The present invention further provides methods and uses for the present probes for detection, imaging, and treatment of other diseases in vivo with the use of a single probe, such as diseases involving inflammation, cardiovascular or neurological diseases.

### VIII. Pharmaceutical Formulations.

Pharmaceutical Formulations.

In certain embodiments the nanoparticle probes of the present invention can be formulated as pharmaceutical compositions (i.e., compositions that are suitable for administration to a subject or patient (i.e., human or non-human subject) that can be used directly and/or in the preparation of unit dosage forms. In certain embodiments, such compositions comprise a therapeutically effective amount of one or more nanoparticle probes (e.g., imaging and/or therapeutic probe) and a pharmaceutically acceptable carrier.

As indicated above, the nanoparticle probes of this invention can be used in a wide variety of contexts including, but not limited to the detection and/or imaging of tumors or cancer cells, inhibition of tumor growth and/or cancer cell growth and/or proliferation, and the like. In certain embodiments one or more antibodies nanoparticle probes can be administered by injection, that is, intravenously, intramuscularly, intracutaneously, subcutaneously, intraduodenally, or intraperitoneally. Also, in certain embodiments, the compounds can be administered by inhalation, for example, intranasally. Additionally, in certain embodiments, the nanoparticle probes can be administered orally, or transdermally or rectally.

In one illustrative embodiment, the term “pharmaceutically acceptable” means approved by a regulatory agency of the Federal or a state government or listed in the U.S. Pharmacopeia or other generally recognized pharmacopeia for use in animals, and more particularly in humans, or suitable for administration to an animal or human. The term “carrier” or refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water or a Ringer's solution is one preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the nanoparticle probes of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments the compositions of the invention can be provided as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Pharmaceutical compositions comprising the nanoparticle probes described herein can be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the nanoparticle probes into preparations that can be used pharmaceutically. Proper formulation is dependent upon the route of administration chosen.

For topical or transdermal administration, the moieties described herein can be formulated as solutions, gels, ointments, creams, lotion, emulsion, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal, inhalation, oral or pulmonary administration. In the context of treatment of neoplasms, intratumoral injections can be performed. One advantageous method for local administration of the described moieties is intracranial infusion by convection-enhanced delivery to the brain.

For injection, the nanoparticle probes described herein can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, compositions comprising the iron chelating agent(s) can be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the nanoparticle probes of this invention can be readily formulated by combining the agent(s) with pharmaceutically acceptable carriers well known in the art. Such carriers enable the agent(s) to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g., lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added.

For buccal administration, the iron chelating agent(s) can take the form of tablets, lozenges, etc. formulated in conventional manner.

For administration by inhalation, the nanoparticle probes of this invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator may be formulated containing a powder mix of the iron chelating agent(s) and a suitable powder base such as lactose or starch.

The nanoparticle probes of this invention (can also be formulated in rectal or vaginal compositions such as suppositories or retention enemas, e.g, containing conventional suppository bases such as cocoa butter or other glycerides.

In addition to the formulations described previously, the nanoparticle probes of this invention can also be formulated as a depot preparation. Such long acting formulations may be administered by implantation (for example subcutaneously or intramuscularly) or by intramuscular injection. Thus, for example, the nanoparticle probes of this invention can be formulated with suitable polymeric or hydrophobic materials (for example as an emulsion in an acceptable oil) or ion exchange resins, or as sparingly soluble derivatives, for example, as a sparingly soluble salt.

Other pharmaceutical delivery systems can also be employed. Liposomes and emulsions are well known examples of delivery vehicles that may be used to deliver the nanoparticle probes of this invention. Certain organic solvents such as dimethylsulfoxide also may be employed, although usually at the cost of greater toxicity. Additionally, the antibodies, and/or functionalized chimeric moieties of this invention can be delivered using a sustained-release system, such as semipermeable matrices of solid polymers containing the therapeutic agent. Various sustained-release materials have been established and are well known by those skilled in the art. Sustained-release capsules may, depending on their chemical nature, can release the active agent(s) for a few days, a few weeks, or up to over 100 days. Depending on the chemical nature and the biological stability of the agent(s) additional strategies for stabilization can be employed.

As nanoparticle probes of this invention may contain charged side chains or termini, they can be included in any of the above-described formulations as the free acids or bases or as pharmaceutically acceptable salts. Pharmaceutically acceptable salts are those salts which substantially retain the biological activity of the free bases and which are prepared by reaction with inorganic acids. Pharmaceutical salts tend to be more soluble in aqueous and other protic solvents than are the corresponding free base forms.

Effective Dosages.

The nanoparticle probes of this invention will generally be used in an amount effective to achieve the intended purpose (e.g., to image a tumor or cancer cell, to inhibit growth and/or proliferation of cancer cells, etc.). In certain preferred embodiments, the nanoparticle probes utilized in the methods of this invention are administered at a dose that is effective to partially or fully inhibit cancer cell proliferation and/or growth, or to enable visualization of a cancer cell or tumor characterized by overexpression of an tumor marker (e.g., an EGF receptor). In certain embodiments, dosages are selected that inhibit cancer cell growth and/or proliferation at the 90%, more preferably at the 95%, and most preferably at the 98% or 99% confidence level. Preferred effective amounts are those that reduce or prevent tumor growth or that facilitate cancer cell detection and/or visualization. With respect to inhibitors of cell growth and proliferation, the compounds can also be used prophalactically at the same dose levels.

Typically, the nanoparticle probes of this invention, or pharmaceutical formulations thereof, are administered or applied in a therapeutically effective amount. A therapeutically effective amount is an amount effective to reduce or prevent the onset or progression (e.g., growth and/or proliferation) of a cancer cell and/or a tumor. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One skilled in the art could readily optimize administration to humans based on animal data.

Dosage amount and interval can be adjusted individually to provide plasma levels of the inhibitors which are sufficient to maintain therapeutic effect.

Dosages for typical therapeutics are known to those of skill in the art. Moreover, such dosages are typically advisorial in nature and may be adjusted depending on the particular therapeutic context, patient tolerance, etc. Single or multiple administrations of the compositions may be administered depending on the dosage and frequency as required and tolerated by the patient.

In certain embodiments, an initial dosage of about 1 μg, preferably from about 1 mg to about 1000 mg per kilogram daily will be effective. A daily dose range of about 5 to about 75 mg is preferred. The dosages, however, can be varied depending upon the requirements of the patient, the severity of the condition being treated, and the compound being employed. Determination of the proper dosage for a particular situation is within the skill of the art. Generally, treatment is initiated with smaller dosages that are less than the optimum dose of the compound. Thereafter, the dosage is increased by small increments until the optimum effect under the circumstance is reached. For convenience, the total daily dosage can be divided and administered in portions during the day if desired. Typical dosages will be from about 0.1 to about 500 mg/kg, and ideally about 25 to about 250 mg/kg.

In cases of local administration or selective uptake, the effective local concentration of the antibodies and/or chimeric moieties may not be related to plasma concentration. One skilled in the art will be able to optimize therapeutically effective local dosages without undue experimentation. The amount of nanoparticle probe will, of course, be dependent on the subject being treated, on the subject's weight, the severity of the affliction, the manner of administration and the judgment of the prescribing physician.

The therapy can be repeated intermittently. In certain embodiments, the pharmaceutical preparation comprising the antibodies and/or chimeric moieties can be administered at appropriate intervals, for example, at least twice a day or more until the pathological symptoms are reduced or alleviated, after which the dosage may be reduced to a maintenance level. The appropriate interval in a particular case would normally depend on the condition of the patient. The therapy can be provided alone or in combination with other drugs, and/or radiotherapy, and/or surgical procedures.

Toxicity.

Preferably, a therapeutically effective dose of nanoparticle probes of the invention described herein will provide therapeutic benefit without causing substantial toxicity.

Toxicity of the nanoparticle probes described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Agents that exhibit high therapeutic indices are preferred. Data obtained from cell culture assays and animal studies can be used in formulating a dosage range that is not toxic for use in human. The dosage of the antibodies, and/or chimeric moieties of this invention preferably lie within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition (see, e.g., Fingl et al. (1975) In: The Pharmacological Basis of Therapeutics, Ch. 1, p. 1).

### IX. Kits.

Where a radioactive, or other, effector is used as a diagnostic and/or therapeutic agent, it is frequently impossible to put the ready-for-use composition at the disposal of the user, because of the often poor shelf life of the radiolabeled compound and/or the short half-life of the radionuclide used. In such cases the user can carry out the labeling reaction with the radionuclide in the clinical hospital, physician's office, or laboratory. For this purpose, or other purposes, the various reaction ingredients can then be offered to the user in the form of a so-called “kit”. The kit is preferably designed so that the manipulations necessary to perform the desired reaction should be as simple as possible to enable the user to prepare from the kit the desired composition by using the facilities that are at his disposal. Therefore the invention also relates to a kit for preparing a composition (e.g., a nanoparticle) according to this invention.

In certain embodiments such a kit according to the present invention comprises a nanoparticle probe described herein. The probe can be provided bearing an imaging agent, and/or a targeting agent, and/or a therapeutic agent, e.g., as described herein. In certain embodiments the probe does not bear the imaging agent, and/or a targeting agent, and/or a therapeutic agent, and the kit typically contains one or more reagents for addition of one or more of these moieties.

In various embodiments the nanoparticle probes are provided, if desired, with inert pharmaceutically acceptable carrier and/or formulating agents and/or adjuvants added. In addition, the kit optionally includes a solution of a salt or chelate of a suitable radionuclide (or other active agent), and, optionally, instructions for use with a prescription for administering and/or reacting the ingredients present in the kit.

In certain embodiments the kit to be supplied to the user may also comprise the nanoparticle probes described above, together with instructions for use, whereas the solution of a salt or chelate of the radionuclide which can have a limited shelf life, can be put to the disposal of the user separately.

The kit can optionally, additionally comprise a reducing agent and/or, if desired, a chelator, and/or instructions for use of the composition and/or a prescription for reacting the ingredients of the kit to form the desired product(s). If desired, the ingredients of the kit may be combined, provided they are compatible.

In certain embodiments, the final nanoparticle probe can simply be produced by combining the components in a neutral medium and causing them to react. For that purpose the imaging agent, and/or targeting agent, and/or therapeutic agent can be presented to the nanoparticle in the form of a chelate.

When kit constituent(s) are used as component(s) for pharmaceutical administration (e.g., as an injection liquid) they are preferably sterile. When the constituent(s) are provided in a dry state, the user should preferably use a sterile physiological saline solution as a solvent. If desired, the constituent(s) can be stabilized in the conventional manner with suitable stabilizers, for example, ascorbic acid, gentisic acid or salts of these acids, or they may comprise other auxiliary agents, for example, fillers, such as glucose, lactose, mannitol, and the like.

While the instructional materials, when present, typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to interne sites that provide such instructional materials.

### EXAMPLES

The following examples are offered to illustrate, but not to limit the claimed invention.

### Example 1

### Paramagnetic Silica-Coated Nanocrystals as an Advanced MRI Contrast Agent

This example describes a robust and general method for embedding nanoparticles, such as quantum dots (QD) or colloidal gold (Au) nanocrystals, into a highly water-soluble thin silica shell doped with paramagnetic gadolinium (Gd³⁺) ions without negatively impacting the optical properties of the QD or Au nanoparticle cores. The ultrathin silica shell has been covalently linked to Gd³⁺ ions chelator, etraazacyclododecanetetraacetic acid (DOTA). The resulting complex has a diameter of 8 to 15 nm and is soluble in high ionic strength buffers at pH values ranging from approximately 4 to 11. For this system, nanoparticle concentrations exceed 50 μM, while most other nanoparticles might aggregate. In magnetic resonance imaging (MRI) experiments at clinical magnetic field strengths of 1.4 T (¹H resonance frequency of 60 MHz), the gadolinium-DOTA (Gd-DOTA) attached to SiO₂-coated QDs has a spin-lattice (T₁) particle relaxivity (r₁) and a spin-spin (T₂) particle relaxivity (r₂) of 1019±19 mM⁻¹s⁻¹ and 2438±46 mM⁻¹s⁻¹, respectively, for a 8-nm QD. The particle relaxivity has been correlated to the number of Gd³⁺ covalently linked to the silica shell. At 1.4 T, the Gd-DOTA ion relaxivities, r₁ and r₂, respectively, are 23±0.40 mM⁻¹s⁻¹ and 54±1.0 mM⁻¹s⁻¹. The sensitivity of our probes is in the 100-nM range for 8-10 nm particles and reaches 10 nM for particles approximately 15 nm in diameter. Preliminary dynamic contrast enhancement MRI experiments in mice revealed that silica-coated MRI probes are cleared from the renal system into the bladder with no observable effects on the health of the animal. This current approach may offer numerous advantages over other approaches (Yang et al. (006) Adv. Mater. 18: 2890; Mulder et al. (2006) Nano Lett. 6: 1) including greater relaxivity and greater simplicity for the synthesis process of dual modality contrast agents that allow both MRI and optical detection as well as applicability to other nanoparticles.

### Experimental Details.

In certain embodiments a three-step process was used to prepare multi-modal probes. First a PEG-ylated silica shell was grown containing thiol groups around the inorganic cores (semiconductor or metallic). Second paramagnetic agents were synthesized made of amine-terminated DOTA molecules that chelatesGd³⁺ ions. Third, the chelated paramagnetic compounds were covalently linked to silanized particles using a bifunctional cross-linker.

Silanization of QDs.

The synthesis and silanization of CdSe/ZnS QDs follow published procedures. Briefly, after a priming step replacing TOPO surfactants on the QD surface with MercaptoPropyltrimethoxySilane (MPS), polymerization of siloxane is performed in methanol under slightly basic conditions. In a second step, addition of fresh MPS and PEG- propyltrimethoxysilane permitted the introduction of functional (SH) and stabilizing (PEG) groups respectively into the forming SiO₂ shell. Silane polymerization was quenched with trimethylchlorosilane that converted reactive silanol groups into methyl groups. This allows controlling and limiting the size of the silica shell to a thickness of a few nanometers. After extensive dialysis against fresh methanol and subsequently against 10 mM phosphate buffer, pH˜7-7.5, silanized QD solutions were concentrated using centricon 100 down to optical densities >30-70 and purified further by low pressure chromatography using a 20 cm long, 1 cm ID column filled with sephadex G200 or sephadex G100. Silanized qdots elute in a rather large band. Typically, we loaded ˜500-700 μl of solution and collected ˜5 ml of solution. Solutions of silanized Qdots were stored at room temperature at optical densities in the range of 3-6.

Silanization of Au Nanoparticles.

A similar approach was used to silanize citrate-stabilized Au nanoparticles of 5 and 10 nm purchased at BBI International. However, in the case of colloidal Au, silanization was performed in an aqueous environment because the particles would not disperse in methanol. Because citrate-coated Au nanoparticles aggregate easily even in low-ionic-strength buffers, the Au colloids were first stabilized with a phosphine surfactant, as we have reported previously (Zanchet et al. (2001) Nano Lett. 1: 32; Loweth et al. (1999) Chem., Int Ed. 38: 1808). Phosphine-stabilized Au colloids were precipitated with ethanol and resuspended into a solution of 1:1000 MPS in water to exchange the capping ligands to a thiolated methoxysilane. After this priming step, an approach similar to the QDs case was taken, which included the growth of a shell using MPS and PEG-silane and quenching of the shell growth using trimethylchlorosilane. All of these steps were performed in water (see, e.g., Gerion et al. ((2007) J. Phys. Chem. C, 111(34): 12542-12551). After the procedure was completed, silanized Au colloids were purified using centrifugation. Silica-coated Au nanocrystals could be concentrated by centrifuging down the solution in a Centricon 100 device to dryness. Upon addition of buffer, the particles were resuspended spontaneously by shaking gently. Such purification was performed several times. Despite these multiple washing steps and large concentrations (optical densities greater than 100), the plasmon peak of silanized Au colloids measured by UV-vis did not shift compared to the original diluted samples for both 5- and 10-nm colloids. Because the plasmon peak of noble metals is very sensitive to particle aggregation, this indicated that silanization of Au colloids yielded well-dispersed nanoparticles (Elghanian et al. (1997) Science 277: 1078-1081; Su et al. (2003) Nano Lett. 3: 1087-1090).

Estimating the Size of the Nanoparticles.

The size estimates of the silanized particles are based on previous AFM investigations. On the basis of those studies, our estimate of the size of the silica shell is about 2-3 nm thick and adds 4-6 nm to the particle diameter (Gerion et al. (2001) J. Phys. Chem. B 105: 8861-8871; Wolcott et al. (2006) J. Phys. Chem. B 110: 5779-5789). In this study, we focused on 5-nm naked CdSe/ZnS, and 5- and 10-nm citrate-stabilized Au colloids. After silanization, the respective sizes were 10 nm for silanized QDs, 10 nm for 5-nm Au, and 16-18 nm for 10-nm Au.

Gd3+ Chelation with a DOTA Moiety.

The synthesis of Gd-DOTA (i.e., one Gd3+ ion chelated by DOTA) was performed according to a procedure adapted from previous reports (Wang et al. (1992) Inorg. Chem. 31: 1095-1099; Prantner et al. (2003) Mol. Imaging 2: 3330-341). We dissolved p-NH₂-Bn-DOTA (2-(4-aminobenzyl)-1,4,7,10-tet-raazacyclododecane-1,4,7,10-tetraacetic acid) in water and add an aqueous solution of GdCl3 (Sigma-Alrich), so as to have a 1:0.98 molar mixture of DOTA/Gd3+ and approximately 0.2 M concentration in DOTA. The solution was heated a few minutes to 80° C. to favor coordination of Gd3+ with the tetraazacy-clododecane ring. The pH of the solution droped below 1. We brought the pH to 3.5-4 by adding aliquots of 7 M NaOH and heat the solution back to 80° C. for a few minutes. At this early stage, heating produces acidification of the solution. Therefore, we repeated heating-adjusting the pH to 3.5-4 with sodium hydroxide several times (up to 7 times), until the heating step did not produce a drop in pH below 3.5. At that stage, the solution was kept at 80° C. for 3 h. Completion of the Gd³⁺ chelation was confirmed by a colorimetric assay using Arsenazo dye (Sigma-Alrich). This colorimetric dye reacts to the presence of unbound or free Gd³⁺. The dye natural color is purple, but if it binds to Gd³⁺ its color turns to blue. After 3 h, the Gd-DOTA solution was slightly yellowish and had a concentration of approximately 150 mM in Gd-DOTA, deduced from the initial amount of DOTA, GdCl₃, and NaOH used. The stability of the Gd-DOTA has been studied using the colorimetric Arsenazo test. No Gd³⁺ release from the DOTA ring was observed over a period of several weeks.

Linking Gd-DOTA to Silanized Nanoparticles.

Freshly prepared paramagnetic Gd-DOTA was covalently linked to silanized nanoparticles to form Gd-DOTA attached to SiO₂-coated QD. First, the amino group on the Gd-DOTA unit is converted into a maleimide group using sulfo-SMCC and classic conjugation conditions (pH approximately 6-6.5, SMCC/DOTA equal to 3:1) (Hermanson (1996) Bioconjugate Techniques; Academic Press Inc.: San Diego, Calif.). After 1 h reaction, the maleimide-activated Gd-DOTA was directly reacted to silanized particles. The reaction was kept running for approximately 24 h at room temperature. Removal of unbound Gd-DOTA was performed by a 48 h dialysis in a 50K MWCO membrane (SpectraPor 6) against a 2 L bath consisting of 10 mM phosphate buffer, pH of 7. We exchanged the buffer bath at least 4 times during the dialysis period. After dialysis, the sample was further purified by centrifuging this solution 4-5 runs with a Centricon 100. For each run, 2 mL of silanized particles were condensed down to less than 100 μL and approximately 1.9 mL of fresh buffer was added. After these extensive purification steps, we estimate that the concentration of unbound Gd-DOTA was in the femtomolar-picomolar range, far too small to provide any signal in MRI and far smaller than a few micromoles, the typical concentration of silanized nanoparticles.

Determination of the Concentration of the Samples.

The concentrations of our solutions are given in terms of silanized nanoparticle concentration and not in terms of Gd³⁺ present in solution because the latter is attached to the nanoparticles. At 1 μM concentration, the average distance between nanoparticles is over 100 nm. We determined the concentration of the nanoparticle solution by measuring the UV-vis spectrum. We deduced the concentration of the solution from the optical density at the exciton (for semiconductors—QDs) or plasmon (for Au colloids) peak using known extinction coefficients and the following equation: C=OD/(εδ), where OD is the optical density or amplitude of absorption at the exciton/plasmon peak, δ is the cuvette length (usually 1 cm or 2 mm), and ε is the extinction coefficient. The extinction coefficients are deduced from the literature (QDs) or given from the manufacturer (Au). We used the following numbers: QD exciton peak at approximately 610 nm, fluorescence emission at approximately 630 nm, full width at half-maximum (fwhm) of 38 nm, extinction coefficient used 620 000 M⁻¹cm⁻¹ following published reports (Yu, et al. (2003) Chem. Mater. 15: 2854-2860). For 5- and 10-nm Au colloids, both plasmon peaks are at 524 nm and we use ε=1.2×10⁷ M⁻¹cm⁻¹ for 5 nm Au and ε=1.06×10⁸ M⁻¹cm⁻¹ for 10-nm Au, respectively. These latter numbers were computed from the concentrations given by the manufacturer and the OD of citrate-stabilized Au colloids measured directly out of the bottle.

Determination of the Number of Gd Per Silanized Nano-Particle.

After extensive purification from unbound Gd-DOTA, these samples were chemically analyzed by inductively coupled plasma mass spectrometry (ICP-MS) by measuring the total amount of Gd and Cd or Au ions. By assuming bulk parameters of the CdSe or Au lattice and the size of the nanoparticles (using tabulated values linking the size of the QDs to its optical properties, or the claimed size for Au nanocrystals), we deduced the number of Gd per silanized nanoparticle. The number of Gd³⁺ per Gd-DOTA attached to SiO₂-coated nanoparticle varied from 3 to greater than 300 and depends on the size of the initial nanoparticles and the conditions used during the conjugation of Gd-DOTA to the silanized nanoparticles. Notice that the same samples were used for MRI study and ICP-MS analysis.

Growing a Nanometer-Thin Silica Shell Around Au Colloids and Other Cores.

The synthesis and use of semiconductor QDs coated with an ultrathin silica shell has been described thoroughly (Gerion et al. (2001) J. Phys. Chem. B 105: 8861-8871; Wolcott et al. (2006) J. Phys. Chem. B 110: 5779-5789). In this example, we have extended the procedure to embed Au colloids of 5- and 10-nm diameter into a thin silica shell. The synthesis of silica shells around Au cores has been detailed in the pioneering work of Liz-Marzan et al. (1996) Langmuir 12: 4329-4335. The authors used a 15-nm Au seed and showed how to grow thick shells (greater than 80 nm) over a period of several days.

Two main issues in growing a silica shell around Au seeds are the avoidance of cross-linking between nanoparticles and the control of the polymerization rate. The latter calls for the use of an anhydrous solvent, whereas the former calls for diluted solutions of nanoparticles. This is because polycondensation of methoxysilane into siloxane bonds is driven by hydrolysis and heat/basicity. Neither of these conditions are desirable. First, citrate-stabilized Au colloids are poorly soluble in solvents other than water (including aqueous buffers). Second, we should dilute 20 mL of as-purchased 5-nm Au colloids (83 nM) in more than 500 mL of water to start with published protocols. Our approach permits us to silanize Au colloids in small volumes (<1-3 mL) at high nanoparticle concentration (greater than 1 μM for 5 nm Au). This procedure is robust and can be applied for the silanization of other inorganic nanoparticle cores such as iron oxide (He et al. (2005) Appl. Phys. 38: 1342-1350).

Our silanization protocol for Au colloids calls for an exchange of the citrate capping ligands with a phosphine stabilizer (bis-(p-sulfonatophenyl)phenylphosphine), as described thoroughly in the literature. Phosphine-stabilized Au colloids are soluble in buffers and water at concentrations 50-100-fold higher than the original one. We silanize the nanoparticles at these high concentrations. (Note: the phosphine capping is just an intermediate step to allow manipulation of Au colloids in water and preventing their aggregation). To grow the silica shell, phosphine groups were replaced with thiolate primers, specifically mercaptopropyltrimethoxysilane or MPS. Because of the strong affinity between thiols and gold surfaces, the capping exchange was fast (less than 20 min) and efficient. The methoxysilane or silanol groups of MPS act as an anchor molecule, upon which the silica shell forms. The consolidation and polymerization of MPS into a siloxane or silica shell can be controlled by choosing weakly alkaline aqueous solutions (pH is approximately 7.5-8) instead of heat. While the shell is slowly forming, fresh MPS and PEG-siloxane are incorporated into the shell. The shell growth is finally quenched by converting the remaining silanol groups into unreactive methyl groups. At this point, the silanized Au colloids can be purified from excess silane by dialysis, repeatedly centrifuged down, and purified with size-exclusion chromatography.

The whole procedure for silanizing Au colloids takes about 3 h and is performed at a particle concentration above 1 μM for 5 nm Au cores and above 0.1 μM for 10 nm cores. We found that the same protocol works for both sizes of Au colloids. In our case, the aggregation of particles during the silanization process does not occur. The plasmon peaks of citrate Au solutions and silanized Au solutions are at the same wavelength (524 and 526 nm, respectively). The UV-vis spectrum of silanized Au solutions is stable for weeks, even though silanized Au solutions are stored at high concentrations in a 10 mM phosphate buffer. Gel electrophoresis mobility of silanized Au is qualitatively similar to that of silanized QD nanoparticles.

20 and 60 MHz NMR Minispec Parameters.

T₁ and T₂ relaxation time measurements were performed on Bruker Minispecs operating at 1H resonance frequencies of 20 and 60 MHz. An inversion recovery pulse sequence was employed for T₁ relaxation time measurements using a monoexponential fit to the recovery curve. For each experiment, 4 scans were collected with a recycle delay of 15 s. To obtain the recovery curve, 50 evenly spaced points were collected with the first point acquired at 5 ms. The last time point was collected at 4000 ms for short T₁ samples and 10,000 ms for samples with long T₁ so that each sample was allowed to fully recover. The receiver gain for each sample was set so that the signal amplitude was approximately 60%.

The T₂ times were calculated from a monoexponential fit to a spin echo decay curve using a Carr-Purcell-Meiboom-Gill (CPMG) pulse sequence. Eight scans were acquired for each experiment with an echo time of 1 ms, a pulse attenuation of 6 dB, and a recycle delay of 3 s. The number of echo times was varied between 100 point, for short T₂, and 3500 point, for long T₂, to acquire the full decay curve for each sample. The receiver gain for each sample was set to the same value that was used in the T₁ experiment.

Bruker 23 mm MicroImaging MRI System Parameters.

All MRI experiments were performed on a Bruker Avance 400 MHz (9.4 T) spectrometer equipped with a high-resolution Micro5 microimaging system with a 25 mm RF coil. To obtain spin-lattice relaxation (T₁) values, a fast imaging with steady-state prećession (FISP) with inversion recovery (IR) sequence was used. The MRI parameters for FISP include an echo time of 1.5 ms, a repetition rate of 3.0 ms, 8 averages, 32 segments, a field of view of 3×3 cm2, a resolution of 234×234 micrometers/pixel, a flip angle (alpha) of 60°, and an inversion delay (T₁) of 235.5 μs. To obtain spin-spin relaxation (T₂) times, we used a multislice multiecho (MSME) sequence. The MRI parameters for MSME including the following: number of echoes, 32; time of echo, 13.8 ms; repetition rate, 10 000 ms; field of view of 3×3 cm²; resolution, 117×117 micrometers/pixel; number of averages, 1; and slice thickness, 1 mm. We also obtained quantitative evaluation of the longitudinal relaxation times, T₁, by plotting M/Mo versus log(τ), that is, the residual magnetization as a function of the recovery time. T₁ values were measured by fitting M(τ)/Mo to an single-exponential growth curve: (M(τ)/Mo)=(1−exp(τ/T₁)). Transverse relaxation times were determined by a plot of log-(M/Mo) versus τ. The T₂ times were computing by fitting log-(M/Mo) to a linear decay curve of log(M/Mo)=τ/T₂.

Mouse Imaging Using a GE 1.5 T MRI System.

All animal studies were conducted under protocols approved by the University of California at San Francisco Institutional Animal Care and Utilization Committee. A 28-week-old TRAMP mouse, which had a catheter in its jugular vein, was anesthetized with isoflurane and maintained at 37° C. with a water blanket. The animal was then placed inside a customized mouse coil within a GE 3T scanner. 2D T₁-weighted baseline images were acquired with 4-mm-thick slices (8-10 slices). The anesthetized mouse was then injected intravenously with about 100 μL of silanized QD colloids with about 200-300 Gd-DOTA per particle (Gd concentration is 50 μM). At 12 and 17 min after injection, T₁-weighted images were again acquired.

### Results.

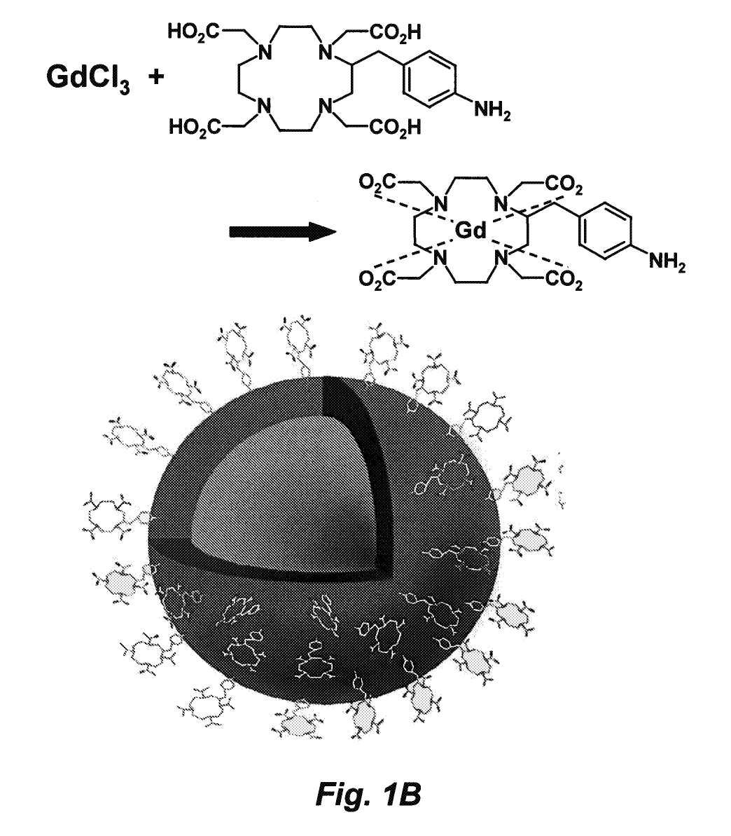

T₁-weighted MRI images of 4 μM Gd-DOTA attached to SiO₂-coated QDs were collected and compared to control solutions (FIG. 2), which include a 4 μM SiO₂-coated QD, 4 μM DOTA attached to SiO₂ with no Gd³⁺, 10 mM phosphate buffer with pH of approximately 7, and Gd-DOTA with various concentrations ranging from 0.39 to 7.35 mM. Qualitative differences in the MRI images clearly showed that Gd-DOTA attached to SiO₂-coated QDs provide a marked contrast compared with the control solutions. This is apparent in examining the T₁ relaxation measurements for the samples shown in FIG. 2. The T₁ relaxation times are summarized in Table 2. The Gd-DOTA attached to SiO₂-coated QDs with a QD concentration of 4 μM had a T₁ relaxation time of 186±3.7 ms, which is significantly shorter than the longitudinal relaxation times of control QD solutions (T₁ of 600±12 ms) and the phosphate buffer solutions (T₁ of 414±8.3 ms). Thus, the chelated Gd-DOTA linked to the silica shell of the QD produces an approximately threefold decrease in T-relaxation time, compared to the T₁ relaxation time of the phosphate buffer.

TABLE 2
T₁ and T₂ relaxation times of the phosphate buffer, SiO₂-coated
QDs, DOTA attached to SiO₂-coated QDs, Gd-DOTA attached to
SiO₂-coated QDs, and various concentrations of Gd-DOTA from
0.39 to 7.35 mM at a 1H Resonance Frequency of 400 MHz
(Data was taken at room temperature.)

| | | |
|---|---|---|
| phosphate buffer | 414 (8.3 | 425 (8.5 |
| SiO2-coated QDs (4 μM) | 600 (12 | 345 (6.9 |
| DOTA attached to | 577 (12 | 347 (6.9 |
| SiO2-coated QDs (4 μM) | | |
| Gd-DOTA attached to | 186 (3.7 | 77 (1.5 |
| SiO2-coated QDs (4 μM) | | |
| Gd-DOTA, 0.39 mM | 237 (4.7 | 201 (4.0 |
| Gd-DOTA, 1.53 mM | 147 (2.9 | 123 (2.5 |
| Gd-DOTA, 7.35 mM | 59 (1.2 | 37 (0.74 |

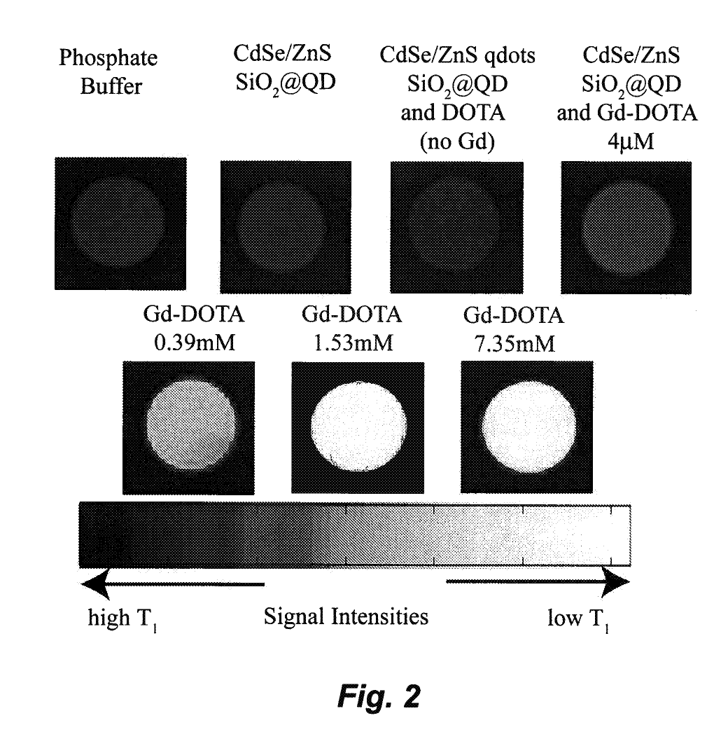

The T₂-weighted MRI images of the same samples are shown in FIG. 3. In these MRI images, Gd-DOTA attached to SiO₂-coated QDs has a clear contrast compared with the control solutions. The T₂ relaxation times are summarized in Table 2. The T2 relaxation time of 4 μM Gd-DOTA attached to SiO₂-coated QDs was 77±1.5 ms, which is significantly shorter than the longitudinal relaxation times of control QD solutions (T₂ of 345±6.9 ms) and the phosphate buffer solutions (T₂ of 425±8.5 ms). In this case, the presence of chelated Gd³⁺ linked to the silica surface produced an approximately fivefold decrease in T₂ compared to control solutions.

From both the T₁-weighted and T₂-weighted images, one can see that the Gd-DOTA attached to SiO₂-coated QD contrast agent acts as both a T₁ and T₂ contrast agent. FIG. 2 demonstrates that the Gd-DOTA attached to SiO₂-coated QDs can be used as a T₁ contrast agent by brightening the image compared with the control samples. FIG. 3 shows that the Gd-DOTA attached to SiO₂-coated QDs can be used as a T₂ contrast agent by causing the image to darken compared to the control samples. Using Gd-DOTA attached to SiO₂-coated QDs as a T₂ contrast agent could be subject to artifacts because the T₂ contrast agent darkens the image rather than brightening the image, this QD contrast agent would be considered to be primarily a T₁ contrast agent. In addition, paramagnetic Gd-DOTA attached to SiO₂-coated QDs provided a contrast in the MRI images, unlike the DOTA only SiO₂-coated QDs and the DOTA-absent SiO₂-coated QDs, which both exhibit similar contrast in the MRI images and the relaxation times to the phosphate buffer. All solutions lacking the paramagnetic Gd³⁺ load exhibit MM images and relaxation times that barely departed from the buffer environment. This observation indicated that the SiO₂-coated QD nanoparticles do not act as T₁ or T₂ contrast agents alone; but the addition of Gd-DOTA attached to the SiO₂-coated QDs creates a MRI T₁ and T₂ contrast agent. Similar qualitative results were observed for Gd-DOTA attached to SiO₂-coated Au nanoparticles, with a nanocrystalline core of 5- and 10-nm diameter. MRI measurements presented so far indicate that Gd-DOTA attached to SiO₂-coated QD and Au nanoparticles, which have a nanoparticle concentration in the micromolar range, has the same T1 and T2 contrast enhancement as the Gd-DOTA with Gd³⁺ concentration in millimolar range.

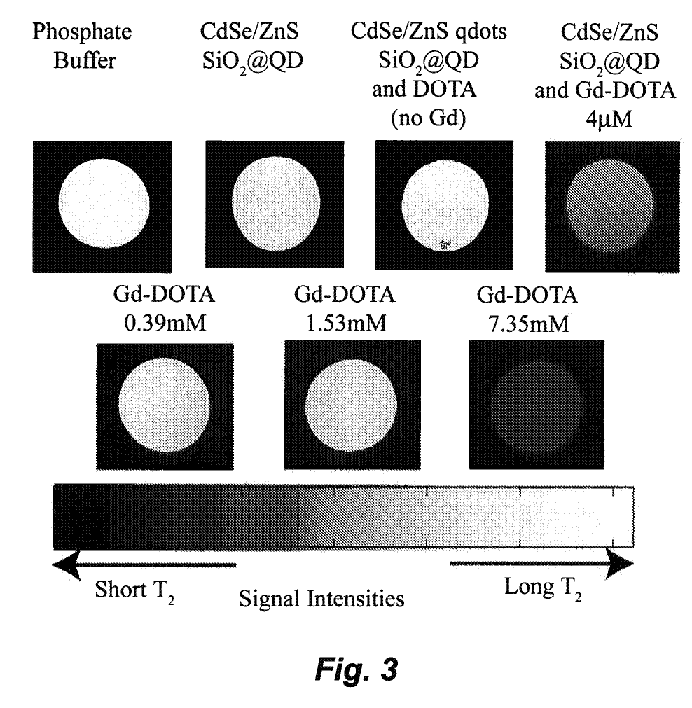

To quantify this increase in contrast enhancement of Gd-DOTA attached to SiO₂-coated QD and Au nanoparticles, we took MRI measurements at various concentrations of contrast agent, and relaxivities were computed. Gd-DOTA attached to SiO₂-coated QDs with nanoparticle concentrations ranging from 0.125 to 4 μM and unbound Gd-DOTA with Gd³⁺ concentrations of 0.39 to 7.125 mM were investigated simultaneously. The T₁ and T₂ maps are shown in FIG. 4A. (Note: the Gd-DOTA with various concentrations is not shown in this figure, but was taken at the same time for a control). In FIG. 3B, we show the linear behaviors of the inverse relaxation times versus the contrast agent concentrations, 1/Ti˜rᵢC, i=1,2. The slopes rᵢ represent the particle relaxivities of Gd-DOTA attached to SiO₂-coated QDs. The Gd³⁺ ion relaxivities of Gd-DOTA attached to SiO₂-coated QDs is shown in brackets, and correspond to r₁=808±15 (18±0.3) mM⁻¹s⁻¹ and r₂=3004±57 (67±1.3) mM⁻¹s'⁻¹ at a 1H resonance frequency of 400 MHz. It is instructive to compare these values with the calculated Gd³⁺ ion relaxivities we measured for unbound Gd-DOTA. These latter ones have r₁=3.7±0.04 mM⁻¹s⁻¹ and r₂=3.8±0.2 mM⁻¹s⁻¹ and are similar to the values found in the literature (Caravan et al. (1999) Chem. Rev. 99: 2293-2352).

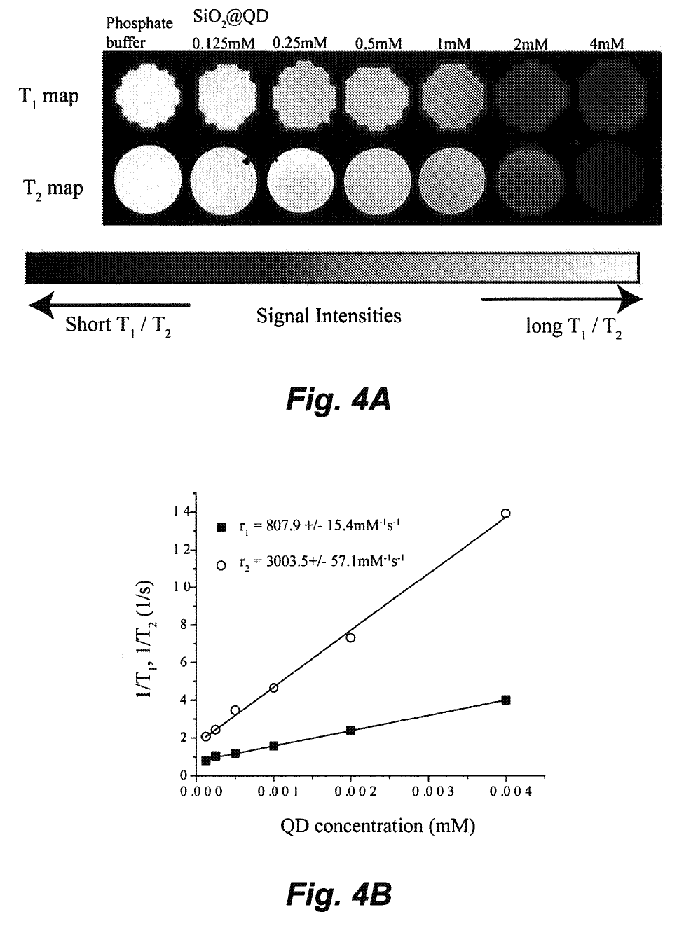

So far, we have focused on the capability of Gd-DOTA attached to SiO₂-coated QDs to enhance the MRI signal using a ¹H resonance frequency of 400 MHz. However, most clinical MRI systems operate at ‘H resonance frequency of 60 MHz or lower frequencies. By examining various concentrations of Gd-DOTA attached to SiO₂-coated QD and Gd-DOTA as a control, the T₁ particle relaxivity (r₁) at ¹H resonance frequencies of 20 and 60 MHz was determined to be r₁=1932±37 mM⁻¹s⁻¹ and r₁=1019±19 mM⁻¹s⁻¹ respectively. The transverse particle relaxivity, r₂, for ¹H resonance frequency of 20 MHz and ¹H resonance frequency of 60 MHz was 2484±47 mM⁻¹s⁻¹ and 2438±46 mM⁻¹s⁻¹. Both r₁ and r₂ particle relaxivities are presented in Table 3 and displayed in FIG. 5. Although we only have three data points, these relaxivity results seem to qualitatively follow the nuclear magnetic relaxation dispersion (NMRD) profile observed for unbound GdDOTA (Aime et al. (1999) Chem. Rev. 321: 185-186; Aime et al. (2002) Magn. Reson. Imaging, 16). In particular, r₁ is strongly field-dependent. It decreases from 1932 mM⁻¹s⁻¹ at ¹H resonance frequency of 20 MHz to 1019 mM⁻¹s⁻¹ at ¹H resonance frequency of 400 MHz. The r₂ relaxivity, as expected, exhibits a very slight increase with increasing fields. However, at all frequencies, the transverse relaxivity of Gd-DOTA attached to SiO₂-coated QDs is larger than the longitudinal relaxivity.

TABLE 3
Nanoparticle relaxivity values at different fields,
which was measured at room temperature.

| frequency | r₁ relaxivity | r₂ relaxivity |
|---|---|---|
| (MHz) | (mM⁻¹s⁻¹⁾ | (mM⁻¹s⁻¹⁾ |
| 20 | 1932 ± 37 | 2484 ± 47 |
| | (43 ± 0.80) | (55 ± 1.1) |
| 60 | 1019 ± 19 | 2438 ± 46 |
| | (23 ± 0.040) | (54 ± 1.0) |
| 400 | 808 ± 15 | 3004 ± 52 |
| | (18 ± 0.030) | (67 ± 1.3) |
| Gd-DOTA | 3-5 | 3-5 |
| 20-100 | | |

(In parenthesis is the value of the Gd³⁺ ion relaxivity. This was calculated from the measurement of Gd concentration per nanoparticle with ICP-MS. The bottom line serves as a comparison with the Gd³⁺ ion relaxivity of unbound Gd-DOTA.)

At the three field strengths investigated here, the particle relaxivities of Gd-DOTA attached to SiO₂-coated QDs reached values over 808 to 1932 mM⁻¹s⁻¹ for r₁ and 3004 to 2484 m⁻¹s⁻¹ for r₂ , whereas the relaxivities of unbound Gd-DOTA only reaches relaxivities of 3-12 mM⁻¹s⁻¹. Through ICP-MS, we determined that there are approximately 45 (15 chelated Gd3+ ions per SiO2-coated QD in the samples presented above. Consequently, at a 1H resonance frequency of 60 MHz for instance, every Gd-DOTA of the 45 Gd-DOTA attached to Si02-coated QDs probe has a Gd3+ ion relaxivity of 23 (0.40 mM−1s−1 and 54 (0.30 mM−1s−1 of r1 and r2, respectively. This represents a 6-fold increase for r1 and 12-fold increase for r2 compared to the value found for unbound Gd-DOTA at the same field strength.

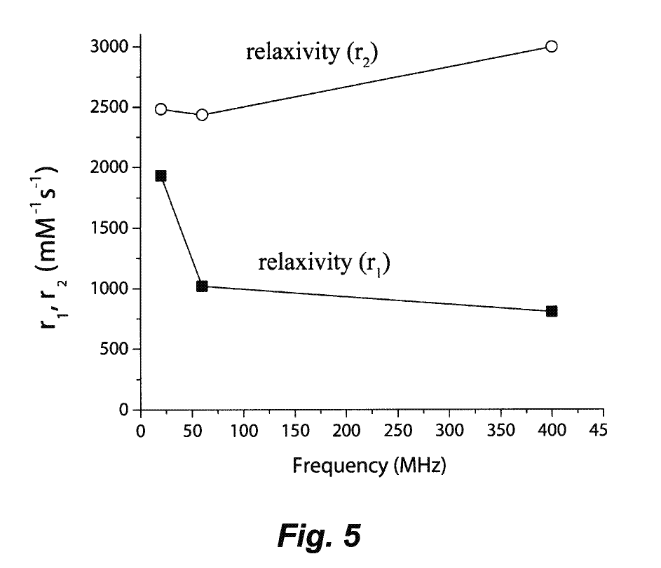

To explore the cause of the increase in ion relaxivity, we initially speculated that the permanent electric dipole of QDs³¹ may affect the local environment that chelated Gd³⁺ experienced through a dipole coupling. This ultimately perturbs the dynamics of water molecules within their environment. To test our assumption, QD nanoparticles were replaced with dipole-free cores such as Au. Thus, we developed the silanization of colloidal gold nanoparticles of 5 and 10 nm diameter. FIG. 6 shows the particle relaxivity of 10-nm Au nanoparticles with a paramagnetic silica shell at ¹H resonance frequency of 20 MHz. The 5-nm Au nanoparticles have the same size as the CdSe ZnS QD cores. At a ¹H resonance frequency of 20 MHz, we measured the particle relaxivities to be r₁=2166±41 mM⁻¹s⁻¹ and r₂=2710±52 mM⁻¹s⁻¹ for silanized 5-nm Au nanoparticles with about 60 Gd-DOTA (determined by ICP-MS). For silanized 10-nm Au with over 320 Gd-DOTA (determined by ICP-MS), we measured r₁=13 510 (250 mM⁻¹s⁻¹ and r₂=15 815±300 mM⁻¹s⁻¹. The particle relaxivity values for Gd-DOTA attached to SiO₂-coated Au with a cores of 5 nm are close to those obtained for Gd-DOTA attached to SiO₂-coated QD solution. In addition, a dramatic increase in particle relaxivity occurs if 10-nm Au nanoparticles are used instead of 5-nm Au cores. These data suggest that the surface area of the nanoparticle rather than the physical nature of the underlying core is the key factor for increased relaxivities.

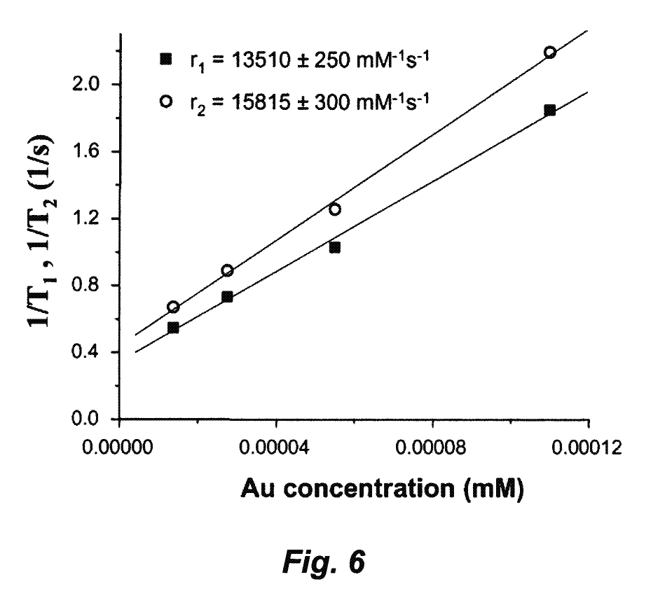

To assess the usefulness of our MRI nanoprobes for in vivo detection, we performed preliminary dynamic contrast-enhanced MRI experiments in a mouse using a 1.5 T scanner. The nanoparticles were administered intravenously to the mouse (100 μL, approximately 50 μM concentration in nanoparticles). Axial sections were collected at 8-s intervals. FIG. 7 represents two T₁-weighted images taken at the level of the bladder before the intravenous injection of the nanoprobe (left) and 5 min after the intravenous injection of the nanoprobe into the mouse (right). The section of the animal is delineated by the thin white line in the left picture. The images have a similar contrast, except at the level of the bladder indicated by the arrow, where the signal increases with time. We attributed it to the clearance of the nanoparticles by the renal system. We observed accumulation of the nanoparticles as early as 300 s after injection. From preliminary data, we observed enhanced contrast in the bladder, possibly in the liver, and a major vein. No contrast enhancement is seen in the kidneys. It is worth noting that there were no acute changes in mouse behavior following the injection of the silica-coated nanoparticles, suggesting an absence of major toxicity.

### Discussion

We have presented a paramagnetic nanoprobe of about 10-15 nm in diameter that consists of an inner inorganic nanoparticle, either CdSe/ZnS QDs or Au, and an ultrathin silica shell, to which chelated paramagnetic ions are covalently linked. To synthesize the silanized CdSe/ZnS QDs or Au nanocrystals coated with Gd-DOTA, a three-step process was used. First, a PEGylated silica shell containing thiol groups around the inorganic cores (semiconductor CdSe/ZnS QDs or metallic Au) was grown. Second, we used a paramagnetic agent made of amine terminated DOTA molecules with chelated Gd³⁺ ions. Third, the chelated paramagnetic compound was covalently crosslinked to silanized particles using a bifunctional cross-linker.

The ability to grow silica shells around inorganic cores has several advantages. First, the nanoparticles are extremely soluble in a wide variety of conditions (i.e., 4<pH<11, and ionic strengths above 1 M of phosphate buffer and 50 mM for buffers with divalent ions). Silanized nanoparticles are also stable in 1×PBS buffer at concentrations exceeding 50 μM. Second, the overall size of the nanoparticles remains small because the silica shell only adds a few nanometers to the particle diameter. We estimate that the silica shell around the 5-nm Au cores is only 2-nm-thick. This results in particle size of about 9 nm. Similarly, we estimate that the silica shell adds about 2-4 nm to Au colloids of 10 nm in diameter, with a resulting total size of 15-18 nm. Finally, conjugation strategies to attach additional molecules to silica are well-developed. This is illustrated by the covalent linking of Gd-DOTA to the silanized nanoparticles. We link together the thiols of the silica shells with amines groups on the DOTA derivative with paramagnetic Gd³⁺ chelated, using the sulfo-SMCC crosslinker (FIG. 1 B). The linking protocol follows closely the one we developed previously to covalently bind DNA to silanized QD (Gerion (2002) Am. Chem. Soc. 124: 7070; Gerion et al. (2002) Chem. Mater. 14: 2113-2119). The design for this MRI contrast agent combined an outer Gd³⁺ paramagnetic shell with an inorganic nanoparticle core. It can generally be described as Gd-DOTA attached to SiO₂-coated QDs or Au. The core provides an optical component (fluorescence or plasmonic), whereas the chelated paramagnetic Gd³⁺ ions linked to the outer shell contribute to MRI relaxivity. The strength of the design consists of the fact that the silica shell does not interfere with optical properties of the inorganic cores. For instance, the position of the plasmon peak of Gd- DOTA attached to SiO₂-coated Au shifts by less than 2 nm compared to citrate-stabilized Au. Similarly, the UV-vis absorption and fluorescence emission of Gd-DOTA attached to SiO₂-coated QDs are virtually similar to those of TOPO- capped QDs. This current approach, where optical and MRI properties arise from physically separated and weakly interacting moieties, presents several advantages over other approaches devised to make multimodality probes.

For an MRI magnetic field strength of 1.4 T (¹H resonance frequency, 60 MHz), the Gd-DOTA attached to SiO₂-coated QDs has spin-lattice and spin-spin particle relaxivities (r₁ and r₂, respectively) of 1019±19 mM⁻¹s^{'11} and 2438±46 mM⁻¹s⁻¹, respectively, for an 8-nm QD. The Gd³⁺ ion relaxivities (based on Gd³⁺ concentration rather than nanoparticle concentration) for the Gd-DOTA attached to SiO₂-coated QDs are 23±0.40 mM⁻¹s⁻¹ and 54±1.1 mM1s⁻¹ r₁ and r₂, respectively, whereas the measured and calculated relaxivity values for the unbound Gd-DOTA were r₁=3.7±0.04 mM⁻¹s⁻¹ and r₂=3.8±0.2 mM⁻s⁻¹. Thus, the Gd³⁺ ion r₁ for Gd-DOTA on SiO₂-coated QDs is increased by approximately 6 times that of Gd-DOTA, and the Gd³⁺ ion r₂ for Gd-DOTA on SiO₂-coated QDs is increased by approximately 14 times that of Gd-DOTA.

Because the Gd-DOTA-SiO₂-Au shows a similar increase in both particle relaxivities and Gd³⁺ ion relaxivities to Gd-DOTA-SiO₂-QD, we concluded that the surface area of the silica shell causes the increase in particle and Gd³⁺ ion relaxivities, rather than the physical nature of the underlying core. Thus, achieving high relaxivities does not require the use of an inorganic core of a specific nature because the MRI contrast enhancement is carried only by the silica shell. In fact, it may be possible to optimize the design and reach even higher relaxivities values by increasing the Gd-DOTA density on the silica shell. Any inorganic core that can be embedded into silica can be used as seed for high-relaxivity contrast agents. This includes any semiconductor nanocrystal, any oxide nanocrystals, and any metal nanocrystals (He et al. (2005) Appl. Phys. 38: 1342-1350).

In this study, we found that Gd-DOTA attached to SiO₂-coated QDs or SiO₂-coated Au with a diameter of about 8-10 nm (5 nm cores+2 nm silica shell) exhibits particle relaxivities of r₁, of approximately 1000-2000 mM⁻¹s⁻¹ and r₂ of approximately 3000 mM⁻¹s⁻¹. Both configurations are detectable at approximately 100 nM concentrations. One reason for this enhanced relaxivity is the number of Gd-DOTA molecules that decorate the silica surface. Chemical analysis indicates that about 45 Gd-DOTA are covering the silica surface of Gd-DOTA-SiO₂-Au with 5-nm cores. More than 250-300 Gd-DOTA were measured around SiO₂-coated Au particles with 10-nm cores. As a result, particle relaxivities of the SiO₂-coated Au particles with 10-nm cores go to approximately 16 000 mM⁻¹s⁻¹ and the detection limit is in the 10 nM range.

Although the number of paramagnetic chelated ions is certainly the major factor in enhancing the particle relaxivities, other effects may also contribute to this enhancement. They manifest themselves by increasing the contribution of every individual Gd-DOTA to the total relaxivity. T₁ and T₂ Gd³⁺ ion relaxivities at 60 MHz are 23 and 54 mM−1s−1 for Gd-DOTA attached to SiO₂-coated QDs and only 3-5 mM⁻¹s⁻¹ for unbound Gd-DOTA. Increased Gd³⁺ ion relaxivities are expected when Gd-DOTA is constrained in its rotational motion. This is observed for macromolecular conjugates (Caravan et al. (1999) Chem. Rev. 99: 2293-2352). It has also been observed in a recent study where paramagnetic lipids were desorbed around a QD nanoparticle and relaxivities in the range of 2000 mM⁻¹s⁻¹ at a 1H resonance frequency of 60 MHz were measured (Mulder et al. (2006) Nano Lett. 6: 1-6). It was determined that approximately 150 Gd-DOTA lipids were surrounding the nanoparticle scaffold, with every Gd³⁺ ion in the lipid payload contributing by about 12 mM⁻¹s⁻¹ to the spin-lattice relaxivity r₁ and by about 18 mM⁻¹s⁻¹ to the spin-spin relaxivity r₂. The authors mainly assume that the increase in Gd³⁺ ion relaxivity came from the reduced tumbling rate of Gd-DOTA because the Gd- DOTA is covalently linking to a higher-molecular-weight macromolecule and the consequent increase in its rotational correlation time.

Our probes are similar to these nanoprobes, except that the paramagnetic lipid coat and the thicker silica layer of 4-7 nm is replaced with a thinner silica shell, which is on the order of 1-2 nm, around the paramagnetic nanoparticle. Yet, this change alone seems to affect the Gd³⁺ ion relaxivities more. Although the rotational correlation time may be slightly different for these two systems, it is unlikely that it alone accounts for this large difference in relaxivity. We believe that a second reason for the increase in Gd³⁺ ion relaxivities is the very hydrophilic environment around Gd-DOTA provided by the silica shell. Because silica is much more hydrophilic than lipids, more protons interact with Gd-DOTA, generating a denser water environment. We rule out that the high permanent electric dipole of the Gd-DOTA attached to SiO₂-coated QDs could affect the dynamics of water proton because the replacement of a QD core with a dipole-free Au particle of similar size produces a similar increase in Gd³⁺ ion relaxivity.

Yang et al. (006) Adv. Mater. 18: 2890, observed similar enhancements with silica-coated CdS:Mn/ZnS with Gd³⁺ functionalized by a metal-chelating silane coupling agent. However, these authors only provide relaxivities at 4.7 T. They also report that the relativity enhancement is due primarily to a reduction in tumbling rates. They indicate that the nanoprobe/MRI contrast agent is primarily a T₂ contrast agent because the r₂/r₁ ratio is 7.4. However, we noticed that the r₂/r₁ ratio is field-dependent; for instance, our measured r₂/r₁ ratio is 3.7 for 9.4 T, 2.3 for 1.41 T, and 1.3 for 0.47 T. If one assumes that a T₁ contrast agent has a preferable r₂/r₁ ratio of 1-2, then at higher frequencies our nanoprobe is more of a T₂ contrast agent, rather than a T₁ contrast agent. However, a T₂ contrast agent could be less-desirable than a T₁ contrast agent, because the T₂ contrast agent darkens the image rather than brightening it.

Impressively, our measured Gd³⁺ ion relaxivities enhancement for Gd-DOTA attached to SiO₂-coated QDs or Au nanoparticles are very close to those obtained for high-generation organic-dendrimer GdDOTA (N>7), where ion relaxivities reach a plateau at 35 and 43 mM⁻¹s⁻¹ respectively (Bryant et al. (1999) J. Magn. Reson. Imaging 9: 348-352). In general, data for Gd-DOTA attached to SiO₂-coated QDs or Au nanoparticles suggests that the MRI relaxivity properties can be described satisfactorily within the framework of the classical relaxation theory (Caravan et al. (1999) Chem. Rev. 99: 2293-2352). Nanoparticles embedded into paramagnetic Gd-DOTA-SiO₂ shells reach particle relaxivities of a few thousand mM⁻¹s⁻¹, and ion relaxivity of a few tens mM⁻¹s⁻¹ (Id.). Gd-DOTA attached to SiO₂-coated QDs surpass the relaxivity of hyperbranched dendrimers of generation N=5 (Langereis et al. (2006) NMR Biomed. 19: 133-141). In fact, our Gd-DOTA attached to SiO₂-coated QDs has particle relaxivities only surpassed by that of the highly branched organic dendrimers of generations N≧7 (Bryant et al. (1999) J. Magn. Reson. Imaging 9: 348-352) and iron oxide nanoparticles with core sizes above 20-40 nm (Gimi et al. (2005) Proc. IEEE 93: 784-799). Our nanoprobes compared favorably with the most-promising new types of MRI contrast agent technology based on ultra-small iron oxide nanoparticles (Song et al. (2005) J. Am. Chem. Soc. 127: 9992-9993; Jun et al.(2005) J. Am. Chem. Soc. 127: 5732-5733; Cho et al. (2006) Nanotechnology 17: 640-644). For instance, a recent report indicated that Au-coated iron oxide nanoparticles with a size of 19 nm have ion relaxivities of only 3 mM⁻¹s⁻¹ in the 30-50 MHz range (Cho et al. (2006) Nanotechnology 17: 640-644). At this size range of 19 nm, the surface chemistry of iron and iron oxide is not yet well-developed. Nanoparticles are often solubilized by ligand exchange (Song et al. (2005) J. Am. Chem. Soc. 127: 9992-9993; Jun et al.(2005) J. Am. Chem. Soc. 127: 5732-5733), although such an approach is unlikely to have widespread use in vivo because of the noncovalent nature of the passivating bonds. Cross-linked, stable, and robust shells are preferably. Silica shells (He et al. (2005) Appl. Phys. 38: 1342-1350), Au shells (Cho et al. (2006) Nanotechnology 17: 640-644), and clustering into polymeric micelles (Ai et al. (2005) Adv. Mater. 17: 12949) have been investigated. However, because of the poor control of surface chemistry, extensive aggregation is often observed for several of these formulations making them unsuitable for in vivo imaging.

Our MRI nanoprobes exhibit a very high solubility and stability. These nanoprobes also represent a compromise between very-high relaxivity values (greater than 100 000 mM⁻¹s⁻¹) obtained with large iron oxide particles (greater than 50-200 nm) and small “protein-like” sizes of branched dendrimers with relaxivities around 1000 mM⁻¹s⁻¹ (Langereis et al. (2006) NMR Biomed. 19: 133-141). In addition, our MRI probes can be made in a few hours in an Eppendorf tube using water as the main solvent and a benchtop centrifuge for purification. The design has considerable potential for scale-up and plenty of room for tailoring the surface to specific biological applications (linking of molecular targeting agents, such as antibodies or small ligand molecules for cell surface receptors).

Bare inorganic nanocrystals tend to aggregate in aqueous solutions and adsorb plasma or other proteins through nonspecific interactions. To prevent their aggregation and tailor their surface properties, nanocrystals must be stabilized and embedded into a biocompatible and robust shell. Silica presents several advantages over polymer-based shells. Unlike polymers, silica neither swells nor changes shape and porosity with changing pHs. Silica is chemically inert and therefore does not influence the redox reaction of the core surface. Furthermore, the chemistry to functionalize silica is well-developed. It is straight-forward to introduce thiols, amine, or carboxylic groups onto a silica surface. The groups can be further derivatized with targeting biomolecules using established conjugation techniques ((1996) Bioconjugate Techniques; Academic Press Inc.: San Diego, Calif.). Finally, it is much easier to control the polymerization of siloxane into silica (and hence the size of the silica shell) than it is to control the thickness of a polymer-based coating. For example, florescence correlation spectroscopy and dynamic light-scattering measurements indicated that although silica-coated 5-nm QDs have a hydrodynamic radius of 8-10 nm, polymer-embedded 5-nm QDs have a hydrodynamic radius close to 30 nm (Doose et al. (2006) Anal. Chem. 77: 2235-2242), and 19-nm Au-coated iron oxide, close to 250 nm (Cho et al. (2006) Nanotechnology 17: 640-644).

Silica has other advantages over polymeric nanoparticles that have emerged in recent live cell studies, including low toxicity. Silica-coated nanoparticles exhibit much-smaller cytotoxicity than polymer-coated nanoparticles (Kirchner et al. (2005) J. Nano Lett. 5: 331-338). Even more remarkable, silica-coated nanoparticles were shown to have negligible perturbation on the gene expression patterns of lung and skin epithelial cells (Zhang et al. (2006) Nano Lett. 6: 800-808). This suggests that silica-coated nanoparticles pose minimal interference with the normal physiology and metabolism of these cell lines.

Toxicity studies at the gene expression level of silica-coated nanoparticles on other cell lines, tissues, or animal models has not been investigated so far and are undoubtedly an emerging research area. Because silica-coated nanoparticles can be functionalized with a wide array of targeting biomolecules (Wolcott et al. (2006) J. Phys. Chem. B 110: 5779-5789; Gerion, D. J. Am. Chem. Soc. 2002, 124, 7070; Gerion et al. (2002) Chem. Mater. 14: 2113-2119), they can be programmed to recognize critical phosphorylation sites, proteases and nucleases, motor proteins, or surface receptors on organelles such as the mitochondria, peroxisome, Golgi body, endosome, and cell nucleus (Chen and Gerion (2004) Nano Lett. 4: 1827-1832). We propose that such nanoparticles may play a key role in cell biology for deciphering molecular pathways.

In a series of early in vivo assays, the contrast agent based on paramagnetic silanized nanoparticles was injected into live mice. We injected about 6×10¹⁵ particles (i.e., ˜200 μL at 50 μM) and did not observe adverse affects on the health of the animals. In fact, the vast majority of silanized particles are excreted into the bladder. We also did not observe a contrast enhancement from other organs, indicating that the large majority of the nanoprobes are cleared by the renal system. The fact that silanized nanoparticles are not taken up by the different organs in a significant manner is a positive sign and indicates the low system toxicity of the nanomaterials. In contrast, the low retention time in the bloodstream represents a problem for many cardiovascular imaging applications. However, both specific uptake and retention time can be implemented or improved by tailoring the surface chemistry of nanoparticles, for instance by grafting of targeting peptides or longer PEG chains.

In desirable embodiments, paramagnetic silica nanoparticles will transverse to the extracellular matrix surrounding blood vessels and microvasculature, recognize cancer cells, and delineate the margin/contour of a tumor. Both size and surface composition will play key roles for such endeavors. Thus, tailoring the surface chemistry of these nanoparticle materials helps facilitate the goal of in vivo imaging of cellular processes. The size of these nanoprobes will play a key role in achieving these goals. Indeed, if the probes are too big, then the nanoprobes will not be able to diffuse effectively into the tumor microvasculature and transverse efficiently and will have limited ability to cross cellular membranes. Although the ideal size of a probe is not known, we hypothesize that ideal probes should be in the 10-15 nm range, as the ones we developed, and have an appropriate surface chemistry. To use our silica-coated nanoparticles for angiogenesis or other targeted applications (stem cell tracking, where a small number of parent cells can be labeled, and guided by surgery), the total relaxivity and contrast enhancement of our probes will preferably be enhanced even further. We estimate that we need to increase the number of Gd-DOTA by a factor of 10 in the QD probes, that is, from 50 to 500, to have sufficient contrast enhancement for certain in vivo applications. Overcoming this challenge seems feasible.

### CONCLUSIONS

We have described a strategy to embed inorganic nanoprobes into a silica shell. The shell is rendered paramagnetic by covalently linking Gd-DOTA to the silica surface of the nanoparticle. Once attached to the surface, each of these contrast agent units exhibit Gd³⁺ ion relaxivities at clinical magnetic fields that are, respectively, approximately 6 times for r₁ and 15 times for r₂ higher than the Gd³⁺ ion relaxivities of unbound Gd-DOTA. We provide evidence that the increase is not related to the nature of the inorganic core but most likely to the fact that Gd-DOTA molecules are attached to a hydrophilic silica surface, which reduces their rotational motion. What matters for imaging purposes is not primarily the Gd³⁺ ion relaxivity of a contrast agent but the particle total relaxivity. Similar to the case of dendrimeric polymers, multiple Gd-DOTA can be anchored onto the surface of a single silica-coated nanoparticle. Because the ion relaxivity is additive, we have measured r₁ and r₂ particles relaxivities at room temperatures and at clinical fields (1.4 T) (r₁ is 1019±19 mM⁻¹s⁻¹ and r₂ is 2438±46 mM⁻¹s⁻¹) for Gd-DOTA attached to SiO₂-coated QDs or Au with particle cores of 5 nm, resulting from the contribution of approximately 45 Gd-DOTA. If the particle cores are 10 nm, then the surface area of silica shells permits the linking of 250-300 Gd-DOTA. Remarkably, these latter probes exhibit relaxivities in excess of 15 000 mM⁻¹s⁻¹ at room temperature and at clinical fields (1.4 T). The silica shell has been demonstrated here to grow around semiconductor and metallic nanoparticles. There are however no restriction in the use of the core material. It may be envisioned to grow a Gd-DOTA-SiO₂ around a supermagnetic core such as small SPIO Fe₃O₄ or Fe₂O₃. In that configuration, perturbation in the dynamic response of water protons will come from the presence of the paramagnetic Gd-DOTA-SiO₂ shell and from the inner SPIO cores. We expect such systems to present even higher relaxivities values than the nanoprobes presented here. In addition, preliminary in vivo tests indicate that paramagnetic silica-coated nanoparticles provide a contrast enhancement in MRI, as evidenced by the signal coming from their accumulation in the bladder. However, higher sensitivities will facilitate obtaining valuable information in MRI on other biological applications.

### Example 2

### Multimodal Probes

A nanoparticle is constructed as illustrated in FIG. 1A. DOTA, anti-PSA/PSMA antibody, and Pc4 are conjugated to the amine group, the thiol group, and the carboxyl group on the Qdot, respectively. The resulting nanocomposite has modalities of MRI, PET, NIR imaging, antibody-based targeting, and photodynamics therapeutics. This nanoconstruct offers sensitive and molecular targeted imaging and imaging-guided intervention. Other modifications include, but are not limited to the conjugation of an enhancer to the photodynamic chemicals, scFv antibodies targeting other prostate cancer antigens identified in the SPORE, and specific peptides/inhibitors against prostate cancer surface antigens.

Construction of the Gd and ⁶⁴Cu-charged DOTA-Qdot (amine group). We have constructed silica-coated Qdots with different surface groups, such as amine, thiol, and carboxyll groups, and can tune the wavelength of Qdot to the absorption window of Pc4. Using EDC-NHS chemistry, we have been able to conjugate DOTA on the surface of Qdots at high density. Gd³⁺ and ⁶⁴Cu²⁺ can be charged into the Qdot-linked DOTA.

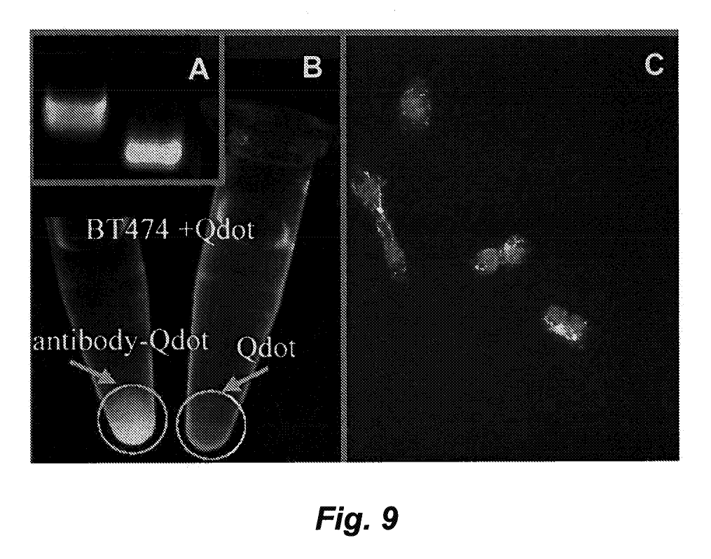

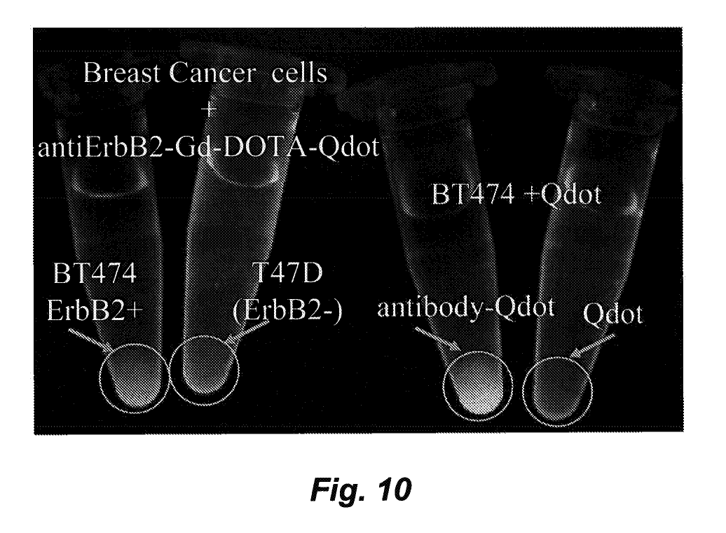

Conjugation of targeting antibody onto the nanoparticle (thiol group). Single chain antibodies (e.g., against PSMA can be obtained). The antibodies are crosslinked to SMCC, a heterobifunctional crosslink that can react with thiol group on Qdot and amine groups on the protein. The antibody-conjugated Qdot is tested for binding activity to PSMA by BiaCore, and uptake by cultured prostate cancer cell lines. FIGS. 9 and 10 show results generated with antibody targeted Qdots

Conjugation of photodynamic therapy reagent phthalocyanine Pc4 onto the nanoparticle (Carboxyl group): Carboxyl groups on the Qdots are activated by EDC-NHS chemistry as described previously. NHS reacts with the amine group on the axial substituent side chain of the phthalocyanine and forms a conjugate. The singlet oxygen generation is measured in vitro, to find the optimal wavelength to use two photon excitation to activate the Pc4 photodynamic process. The nanocomposite is then targeted to the prostate cancer cells that are overexpressing PSMA. Tests on cancer killing can be measured in a xenograft mouse model.

### Example 3

### Targeting Breast And Prostate Cancer In Vivo With Multimodal Probes

The multimodal nanoparticle probe described in Example 2 is used to target cancer in vivo. To target prostate tumors, it is possible to utilize two scFvs. One scFv stains primary and metastatic tissue and binds to MEMD (CD166). MEMD has recently been found to be overexpressed in up to 84% of prostate cancer. The second scFv can e A33 which binds an unknown prostate tumor antigen. A33 has exquisite specificity for metastatic prostate tissue

Although there is still some controversy about prostate cancer screening in asymptomatic men, several organizations have formally made recommendations for serum Prostate Specific Antigen (PSA) testing. PSA screening appears to reduce the number of prostate cancers detected at late stage, and the incidence of metastatic prostate cancer. However, because PSA-screening does not distinguish between benign and malignant prostatic disease, a large fraction of biopsies that are performed are unnecessary. Therefore the question arises whether it is possible to avoid unnecessary biopsies and reduce substantial costs. Thus, development of new, imaging-based, non-invasive tests that utilize prostate cancer-specific markers could significantly reduce the need for biopsy, and the attendant cost and morbidity. In addition, if an activatable therapeutic agent can be coupled to the imaging modality, greater precision and discrimination in treatment could be achieved.

For breast cancer, ErbB2 (overexpressed in >35% of breast tumor) can be targeted. ErbB2 overexpressing tumors are a subset of breast cancers with a poor prognosis, and thus being able to image such tumors is of clinical relevance. For prostate cancer, MEMD(CD166) (>84% of prostate tumor) can be targeteed. MEMD overexpression has recently been found to correlate with shortened survival.

Antibodies against Her2, MEMD(CD166)(H3), and antibody A33 can be obtained (see, e.g., PCT Publication WO 2005/062977, and copending U.S. Application 60/973,005, and Scott et al. (2005) Clin. Cancer Res., 11: 4810-4817). The antibody is conjugated to Qdot with SMCC crosslinker. The antibody-conjugated Qdot can be tested for binding activity to Her2 and MEMD(CD 166) proteins by BiaCore, and uptake by cultured breast and prostate cancer cell lines overexpressing the targeted antigens (e.g., as in FIG. 9).

To target ErbB2, we will utilize the scFv C6ML3-9 can be used which binds ErbB2 with a K_{D} of 1.0×10⁻⁹ M (see, e.g., Schier et al., (1995) Immunotechnology 1: 73-81). C6ML3-9 is an affinity matured version of C6.5, a fully human scFv isolated from a phage antibody library. Both C6.5 and C6ML3-9 specifically bind to ErbB2 and localize in ErbB2 expressing SK-OV-3 xengrafts in nude mice. The higher affinity C6ML3-9 results in increased uptake in tumor xenografts in vivo compared to the lower affinity C6.5, as described in Adams et al. (1998) Cancer Res 58:485-490. A C6.5 diabody, an scFv dimer generated by shortening the peptide linker between the immunoglobulin V_{H} and V_{L} domains can also be used. The bivalent diabodies have significantly higher functional affinities than scFv and increased tumor localization in vivo (see, e.g., Adams et al. (1998) Br. J. Cancer 77: 1405-1412; Nielsen et al. (2000) Cancer Res 60: 6434-6440; Robinson et al. (2005) Cancer Res 65: 1471-1478).

Mice can be used for evaluation of multimodal-Qdot agents (nanoparticle probes). For the initial studies, these can consist of non-tumor bearing mice that are used to characterize system sensitivity and specificity. In follow up studies, mice are generated that bear non-identical subcutaneous human xenograft tumors, one on each flank. In order to provide an internal control, one of the tumors chosen is one that is known to express the targeting antigen and the contralateral tumor will be one that lacks the antigen. Human tumor xenografts carrying BT474, MCF10A (breast), PC3 and DU-145 and LN-CaP (prostate) are generated in nude or SCID mice either as subcutaneous implants or into orthotopic sites, including mammary fat pad, intra-peritoneal, intra-osseous, intra-cardiac, intra-pancreas, and kidney capsule. Tumor cell xenografts are selected based upon the genomic and phenotypic features desired for hypothesis testing and agent evaluation. This is particularly relevant for the targeted nanoparticles since predictions can be made and tested based on known cellular receptor/antigen status. On the basis of previous imaging studies, the initial tumor size is selected to be from 100-400 mm³, although smaller tumors and disseminated tumors can also be investigated. In one stage of the analysis intravenous and intracardiac injection of tumor cells are used, and the sensitivity of the Q-dot imaging agent is determined. In this case, cell lines (which, as above are characterized with respect to the targeting antigen) are administered in such a manner as to allow disseminated localization, including lung, liver, spleen and bone. To aid in their identification and to allow cross-validation of the multimodal-Qdot imaging approach, the cells used are stably expressing a triple reporter vector.

This allows detection by bioluminescent noninvasive whole animal imaging as well post-euthanasia fluorescent tumor cell detection. Sets of mice are prepared for both breast cell lines, and the three prostate cell lines for which the targeting antibody will by the Her2Neu antigen, and for prostate cell lines for which the targeting antibody will be against one of the novel prostate specific antigens.

The biostability of the nanoparticle can be assessed by measuring full body fluorescence postmortem over different time points. The impact of the nanoparticle upon biodistribution of the antibodies can also be evaluated. Biodistribution studies using ¹²⁵I-labeled antibodies have been performed for a number of antibody constructs (scFvs, diabodies) and antibody-liposome conjugates. The same approach can be taken here.

Briefly, two days prior to injection with the ¹²⁵I labeled anti-ErbB2 or other nanoparticles, Lugol's solution is placed in the drinking water to block thyroid accumulation of radioiodine. The nanoparticle is then injected into the tail vein of cohorts of five to six mice. Injected doses are determined by counting the mice on a Series 30 multichannel analyzer/probe system. The cohort is sacrificed at set times after injection and tumor organ and blood retentions are determined by established methods, described in Adams et al. (1998) Br. J. Cancer 77: 1405-1412. Optimal time frames, doses, and antibody constructs for optimal delivery of the anti-ErbB2 nanoparticle can be verified by this well established method. Dose escalation can be performed to determine the toxic dose for the animal.

The antibody-multimodal-Qdot is injected through tail vein into nude mice carrying human breast cancer and prostate cancer xenograft. The mice are imaged with both MRI and NIR imaging under situations simulating surgery for breast cancer and seed placement for prostate cancer. MRI is performed by the at variable time intervals after intravenous injection of the anti-ErbB2 multi-modality nanoparticle into animals (n=10). Similarly, optical imaging is performed using CRI's Maesro In-Vivo Imaging System at variable time periods after injection. Maximum intensity projection (MIP) image of the biodistribution of the multimodal ⁶⁴Cu-nanoparticles for in vivo PET imaging are carried out.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A probe comprising a nanoparticle coated with a hydrophilic coating attached to an imaging agent.

2. The probe of claim 1, wherein said hydrophilic coating comprises one or more materials selected from the group consisting of poly(ethylene glycol) (PEG), polyethylene glycol copolymer, and silica.

3. The probe of claim 1, wherein said hydrophilic coating comprises silica.

4. The probe of claim 1, wherein said hydrophilic coating comprises poly((3-trimethoxysilyl)propyl methacrylate-r-poly(ethylene glycol) methyl ether methacrylate) (poly(TMSMA-r-PEGMA).

5. The probe of claim 1, wherein said hydrophilic coating comprises a methacrylate-based comb polymer containing pendant oligoethylene glycol side chains.

6. A probe comprising a nanoparticle coated with a substantially transparent coating, attached to an imaging agent.

7. The probe of claim 6, wherein said substantially transparent coating comprises silica.

8. A probe comprising a nanoparticle attached to an MRI contrast agent wherein said probe has a T₁ and/or T₂ relaxivity of greater than 200 mM⁻¹ s⁻¹ at clinical field strength.

9. The probe of claim 8, wherein said probe has a T₁ and/or T₂ relaxivity of greater than about 1000 mM⁻¹ s⁻¹.

10. The probe of claim 8, wherein said probe has a T₁ and/or T₂ relaxivity of greater than about 2000 mM⁻¹ s⁻¹.

11. The probe of claim 8, wherein said probe has a T₁ and/or T₂ relaxivity of greater than about 10,000 mM⁻¹ s⁻¹.

12. The probe according to claim 8, wherein said nanoparticle is coated with a coating comprising silica and/or a polymer.

13. The probe according to claim 12, wherein said coating comprises silica.

14. The probe according to any of claims 3, 7, and 13, wherein said silica comprises SiO₂.

15. The probe according to any of claims 1-14, wherein said nanoparticle comprises an inorganic material.

16. The probe of claim 15 wherein said nanoparticle comprises a quantum dot.

17. The probe of claim 16, wherein said nanoparticle is capable of emitting in the visible region of the spectrum.

18. The probe of claim 16, wherein said nanoparticle is capable of emitting in the near infra-red region of the spectrum.

19. The probe of claim 16, wherein said nanoparticle is capable of emitting in the ultraviolet region of the spectrum.

20. The probe of claim 15, wherein said nanoparticle comprises a material selected from the group consisting of an element of Groups II-VI, a semiconductor of Groups II-VI, an oxide or nitride of the element or semiconductor of Groups II-VI.

21. The probe of claim 15 or 16, wherein said nanoparticle has a core having the formula MX, where:
M is one or more materials selected from the group consisting of cadmium, zinc, magnesium, mercury, aluminum, gallium, indium, and thallium; and
X is one or more materials selected from the group consisting of oxygen, sulfur, selenium, tellurium, nitrogen, phosphorus, arsenic, and antimony.

22. The probe of claim 15 or 21, wherein said nanoparticle comprises a core and a shell, wherein said shell comprises a semiconductor overcoating said core.

23. The probe of claim 22, wherein said shell comprises a group II, III, IV, V, or VI semiconductor.

24. The probe of claim 22, wherein said shell comprises one or more materials selected from the group consisting of ZnO, ZnS, ZnSe, ZnTe, CdO, CdS, CdSe, CdTe, MgO, MgS, MgSe, MgTe, HgO, HgS, HgSe, HgTe, AlN, AlP, AlAs, AlSb, GaN, GaP, GaAs, GaSb, InN, InP, InAs, InSb, TlN, TlP, TlAs, and TlSb.

25. The probe of claim 15, wherein said nanoparticle comprises a CdSe core and a ZnS shell and an SiO₂ hydrophilic coating.

26. The probe of claim 15, wherein said nanoparticle has a characteristic dimension of less than about 30 nm.

27. The probe according to any of claims 1-26, wherein said imaging agent comprises one or more agents selected from the group consisting of a magnetic resonance (MRI) imaging agent, a positron emission (PET) imaging agent, an electron spin resonance (ESR) imaging agent, and a near infrared (NIR) imaging agent.

28. The probe of claim 27, wherein said imaging agent comprises an MRI contrast agent comprising a material selected from the group consisting of gadolinium, xenon, iron oxide, and copper.

29. The probe of claim 27, wherein said imaging agent comprises a PET imaging agent comprising a label selected from the group consisting of ¹¹C, ¹³_{N,} ¹⁸F, ⁶⁴Cu, ⁶⁸Ge, and ⁸²Ru.

30. The probe of claim 29, wherein the imaging agent is a PET imaging agent selected from the group consisting of [¹¹C]choline, [¹⁸F]fluorodeoxyglucose(FDG), [¹¹C]methionine, [¹¹C]choline, [¹¹C]acetate, [¹⁸F]fluorocholine, and [¹⁸F]polyethyleneglycol stilbenes.

31. The probe of claim 27, wherein said imaging agent comprises one or more agents selected from the group consisting of a cyanine derivative, and an indocyanine derivative.

32. The probe of claim 31, wherein said imaging agent comprises an agent selected from the group consisting of Cy5.5, IRDye800, indocyanine green (ICG), and an indocyanine green derivative.

33. The probe according to claim 27, wherein said imaging agent comprises an electron spin resonance agent comprising a paramagnetic or superparamagnetic material.

34. The probe according to claim 33, wherein said imaging agent comprises a yttrium iron garnet.

35. The probe according to claim 27, wherein said imaging agent is attached to the nanoparticle by a linker.

36. The probe according to claim 35, wherein said linker comprises a chelating agent.

37. The probe according to claim 35, wherein said linker comprises DOTA.

38. The probe according to any one of claims 1-37, wherein said probe further comprises a targeting moiety attached to said nanoparticle.

39. The probe according to claim 38, wherein said targeting moiety comprises one or more moieties selected from the group consisting of a nucleic acid, a peptide, an enzyme, a lipid, an antibody, a polysaccharide, a lectin, a selectin, a sugar, an aptamers, a drug, and a receptor ligand.

40. The probe according to claim 38, wherein said targeting moiety is an antibody that binds an antigen selected from the group consisting of, a gastrointestinal cancer cell surface antigen, a lung cancer cell surface antigen, a brain tumor cell surface antigen, a glioma cell surface antigen, a breast cancer cell surface antigen, an esophageal cancer cell surface antigen, a common epithelial cancer cell surface antigen, a common sarcoma cell surface antigen, an osteosarcoma cell surface antigen, a fibrosarcoma cell surface antigen, a melanoma cell surface antigen, a gastric cancer cell surface antigen, a pancreatic cancer cell surface antigen, a colorectal cancer cell surface antigen, a urinary bladder cancer cell surface antigen, a prostatic cancer cell surface antigen, a renal cancer cell surface antigen, an ovarian cancer cell surface antigen, a testicular cancer cell surface antigen, an endometrial cancer cell surface antigen, a cervical cancer cell surface antigen, a Hodgkin's disease cell surface antigen, a lymphoma cell surface antigen, a leukemic cell surface antigen and a trophoblastic tumor cell surface antigen.

41. The probe according to claim 38, wherein said targeting moiety is an antibody that binds an antigen selected from the group consisting of 5 alpha reductase, α-fetoprotein, AM-1, APC, APRIL, BAGE, β-catenin, Bc12, bcr-abl (b3a2), CA-125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, Cox-2, DCC, DcR3, E6/E7, EGFR, EMBP, Ena78, FGF8b and FGF8a, FLK-1/KDR, Folic Acid Receptor, G250, GAGE-Family, gastrin 17, GD2/GD3/GM2, GnRH, GnTV, gp100/Pmel17, gp-100-in4, gp15, gp75/TRP-1, hCG, Heparanase, Her2/neu, HER3, Her4, HMTV, HLA-DR10, Hsp70, hTERT , IGFR1, IL-13R, iNOS, Ki 67, KIAA0205, K-ras, H-ras, N-ras, KSA, (CO17-1A), LDLR-FUT, MAGE Family (MAGE1, MAGE3, etc.), Mammaglobin, MAP17, Melan-A/, MART-1, mesothelin, MIC A/B, MT-MMP's, such as MMP2, MMP3, MMPI, MMP9, Mox1, MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin , PSA, PSM, RAGE-1, Rb, RCAS1, SART-1, SSX gene, family, STAT3, STn, TAG-72, TGF-α, TGF-β, and Thymosin β15, nucleolin, Ca15-3, astro Intestinal Tumor Antigen (Ca19-9), ovarian Tumor Antigen (Ca125), Tag72-4 Antigen (CA72-4) and carcinoembryonic antigen (CEA).

42. The probe according to any one of claims 1-41, wherein said probe further comprises a therapeutic moiety attached to said silica-coated nanoparticle.

43. The probe of claim 42, wherein said therapeutic moiety comprises one or more moieties selected from the group consisting of a photosensitizer, a radiosensitizer, an ESR heating moiety, an isotope, a cytotoxin, and a cancer drug.

44. The probe of claim 43, wherein said therapeutic moiety comprises an isotope selected from the group consisting of ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁵²Fe, ^{52m}Mn, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag.

45. The probe of claim 43, wherein said therapeutic moiety comprises an isotope that is a gamma emitter.

46. The probe of claim 43, wherein said therapeutic moiety comprises a photosensitizer selected from the group consisting of a haematoporphyrin derivative, photophrin II, a benzoporphyrins, a tetraphenyl porphyrin, a chlorine, and a phthalocyanine.

47. The use of a probe according to any of claims 1-46 in the manufacture of a medicament for the detection and/or treatment of a cancer.

48. A method of making a nanoprobe, said method comprising:
forming a silica shell around a nanoparticle;
chelating a paramagnetic or superparamagnetic compound; and
coupling the chelated compound to said silica shell thereby forming a nanoprobe.

49. The method of claim 48, further comprising attaching a targeting moiety to said nanoprobe.

50. The method of claim 48 or 49, further comprising attaching a therapeutic moiety to said nanoparticle.

51. A method of detecting a cancer cell, said method comprising:
contacting said cell with a probe comprising a nanoparticle coated with a hydrophilic coating attached to a targeting moiety and an imaging agent, whereby said probe preferentially associates with a cancer cell; and
detecting said imaging agent thereby providing an indication of the presence and/or location of said cancer cell.

52. The method of claim 51, wherein said contacting comprises a modality selected from the group consisting of systemic administration to a mammal, local administration to a tumor or tumor site, administration to a surgical site, ex vivo administration to a sample, and in situ administration to a histological preparation.

53. The method of claim 51, wherein said cancer cell is a cell in a solid tumor.

54. The method of claim 51, wherein said cancer cell is a metastatic cell.

55. The method of claim 51, wherein said cell is a cancer cell in a human.

56. The method of claim 51, wherein said cell is a cancer cell in a non-human mammal.

57. The method of claim 51, wherein said hydrophilic coating comprises one or more materials selected from the group consisting of poly(ethylene glycol) (PEG), polyethylene glycol copolymer, and silica.

58. The method of claim 51, wherein said hydrophilic coating comprises silica.

59. The method of claim 51, wherein said hydrophilic coating comprises poly((3-trimethoxysilyl)propyl methacrylate-r-poly(ethylene glycol) methyl ether methacrylate) (poly(TMSMA-r-PEGMA).

60. The method of claim 51, wherein said hydrophilic coating comprises a methacrylate-based comb polymer containing pendant oligoethylene glycol side chains.

61. The method according to any of claims 51-60, wherein said nanoparticle comprises an inorganic material.

62. The method of claim 61, wherein said nanoparticle comprises a quantum dot.

63. The method of claim 61, wherein, said nanoparticle is capable of emitting in the visible region of the spectrum, said nanoparticle is capable of emitting in the near infra-red region of the spectrum, and/or said nanoparticle is capable of emitting in the ultraviolet region of the spectrum.

64. The method of claim 61, wherein said nanoparticle comprises a material selected from the group consisting of an element of Groups II-VI, a semiconductor of Groups II-VI, an oxide or nitride of the element or semiconductor of Groups II-VI.

65. The method of claim 61, wherein said nanoparticle has a core having the formula MX, where:
M is one or more materials selected from the group consisting of cadmium, zinc, magnesium, mercury, aluminum, gallium, indium, and thallium; and
X is one or more materials selected from the group consisting of oxygen, sulfur, selenium, tellurium, nitrogen, phosphorus, arsenic, and antimony.

66. The method of claim 61, wherein said nanoparticle comprises a core and a shell, wherein said shell comprises a semiconductor overcoating said core.

67. The method of claim 66, wherein said shell comprises a group II, III, IV, V, or VI semiconductor.

68. The method of claim 66, wherein said shell comprises one or more materials selected from the group consisting of ZnO, ZnS, ZnSe, ZnTe, CdO, CdS, CdSe, CdTe, MgO, MgS, MgSe, MgTe, HgO, HgS, HgSe, HgTe, AlN, AlP, AlAs, AlSb, GaN, GaP, GaAs, GaSb, InN, InP, InAs, InSb, TlN, TlP, TlAs, and TlSb.

69. The method of claim 66, wherein said nanoparticle comprises a CdSe core and a ZnS shell and an SiO₂ hydrophilic coating.

70. The method of claim 61, wherein said nanoparticle has a characteristic dimension of less than about 30 nm.

71. The method according to any of claims 51-70, wherein said imaging agent comprises one or more agents selected from the group consisting of a magnetic resonance (MRI) imaging agent, a positron emission (PET) imaging agent, an electron spin resonance (ESR) imaging agent, and a near infrared (NIR) imaging agent.

72. The method of claim 71, wherein said imaging agent comprises an MRI contrast agent comprising a material selected from the group consisting of gadolinium, xenon, iron oxide, and copper.

73. The method of claim 71, wherein said imaging agent comprises a PET imaging agent comprising a label selected from the group consisting of ¹¹C, ¹³N, ¹⁸F, ⁶⁴Cu, ⁶⁸Ge, and ⁸²Ru.

74. The method of claim 71, wherein the imaging agent is a PET imaging agent selected from the group consisting of [¹¹C]choline, [¹⁸F]fluorodeoxyglucose(FDG), [¹¹C]methionine, [¹¹C]choline, [¹¹C]acetate, [¹⁸F]fluorocholine, and [¹⁸F]polyethyleneglycol stilbenes.

75. The method of claim 71, wherein said imaging agent comprises one or more agents selected from the group consisting of a cyanine derivative, and an indocyanine derivative.

76. The method of claim 71, wherein said imaging agent comprises an electron spin resonance agent comprising a paramagnetic or superparamagnetic material.

77. The method of claim 71, wherein said imaging agent is attached to the nanoparticle by a linker.

78. The method of claim 71, wherein said linker comprises a chelating agent.

79. The method according to any one of claims 51-78, wherein said targeting moiety comprises one or more moieties selected from the group consisting of a nucleic acid, a peptide, an enzyme, a lipid, an antibody, a polysaccharide, a lectin, a selectin, a sugar, an aptamers, a drug, and a receptor ligand.

80. The method according to claim 79, wherein said targeting moiety is an antibody that binds an antigen selected from the group consisting of, a gastrointestinal cancer cell surface antigen, a lung cancer cell surface antigen, a brain tumor cell surface antigen, a glioma cell surface antigen, a breast cancer cell surface antigen, an esophageal cancer cell surface antigen, a common epithelial cancer cell surface antigen, a common sarcoma cell surface antigen, an osteosarcoma cell surface antigen, a fibrosarcoma cell surface antigen, a melanoma cell surface antigen, a gastric cancer cell surface antigen, a pancreatic cancer cell surface antigen, a colorectal cancer cell surface antigen, a urinary bladder cancer cell surface antigen, a prostatic cancer cell surface antigen, a renal cancer cell surface antigen, an ovarian cancer cell surface antigen, a testicular cancer cell surface antigen, an endometrial cancer cell surface antigen, a cervical cancer cell surface antigen, a Hodgkin's disease cell surface antigen, a lymphoma cell surface antigen, a leukemic cell surface antigen or a trophoblastic tumor cell surface antigen.

81. The method according to claim 79, wherein said targeting moiety is an antibody that binds an antigen selected from the group consisting of 5 alpha reductase, α-fetoprotein, AM-1, APC, APRIL, BAGE, β-catenin, Bc12, bcr-abl (b3a2), CA-125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, Cox-2, DCC, DcR3, E6/E7, EGFR, EMBP, Ena78, FGF8b and FGF8a, FLK-1/KDR, Folic Acid Receptor, G250, GAGE-Family, gastrin 17, GD2/GD3/GM2, GnRH, GnTV, gp100/Pmel17, gp-100-in4, gp15, gp75/TRP-1, hCG, Heparanase, Her2/neu, HER3, Her4, HMTV, HLA-DR10, Hsp70, hTERT , IGFR1, IL-13R, iNOS, Ki 67, KIAA0205, K-ras, H-ras, N-ras, KSA, (C017-1A), LDLR-FUT, MAGE Family (MAGE1, MAGE3, etc.), Mammaglobin, MAP17, Melan-A/, MART-1, mesothelin, MIC A/B, MT-MMP's, such as MMP2, MMP3, MMP7, MMP9, Mox1, MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin , PSA, PSM, RAGE-1, Rb, RCAS1, SART-1, SSX gene , family, STAT3, STn, TAG-72, TGF-α, TGF-β, and Thymosin β 15, nucleolin, Ca15-3, astro Intestinal Tumor Antigen (Ca19-9), ovarian Tumor Antigen (Ca125), Tag72-4 Antigen (CA72-4). and carcinoembryonic antigen (CEA).

82. The method of claim51, wherein said probe further comprises a therapeutic moiety attached to said silica-coated nanoparticle.

83. The method of claim 82, wherein said therapeutic moiety comprises one or more moieties selected from the group consisting of a photosensitizer, a radiosensitizer, an ESR heating moiety, an isotope, a cytotoxin, and a cancer drug.

84. A method of inhibiting the growth and/or proliferation of a cancer cell, said method comprising:
contacting said cell with a probe comprising a nanoparticle coated with a hydrophilic coating attached to a targeting moiety, an imaging agent, and a therapeutic moiety whereby said probe preferentially associates with a cancer cell and inhibits the growth and/or proliferation of said cell.

85. The method of claim 84, wherein said contacting comprises a modality selected from the group consisting of systemic administration to a mammal, local administration to a tumor or tumor site, and administration to a surgical site.

86. The method of claim 84, wherein said contacting comprises administering said probe to a mammal via a modality selected from the group consisting of oral administration, nasal administration, topical administration, transdermal administration, rectal administration, systemic administration, and administration directly to a tumor or tumor site.

87. The method of claim 84, wherein said cancer cell is a cell in a solid tumor.

88. The method of claim 84, wherein said cancer cell is a metastatic cell.

89. The method of claim 84, wherein said cell is a cancer cell in a human.

90. The method of claim 84, wherein said cell is a cancer cell in a non-human mammal.

91. The method of claim 84, wherein said hydrophilic coating comprises one or more materials selected from the group consisting of poly(ethylene glycol) (PEG), polyethylene glycol copolymer, and silica.

92. The method of claim 84, wherein said nanoparticle comprises an inorganic material.

93. The method of claim 84, wherein said nanoparticle comprises a quantum dot.

94. The method of claim 84, wherein, said nanoparticle is capable of emitting in the visible region of the spectrum, said nanoparticle is capable of emitting in the near infra-red region of the spectrum, and/or said nanoparticle is capable of emitting in the ultraviolet region of the spectrum.

95. The method of claim 84, wherein said nanoparticle comprises a material selected from the group consisting of an element of Groups II-VI, a semiconductor of Groups II-VI, an oxide or nitride of the element or semiconductor of Groups II-VI.

96. The method of claim 84, wherein said nanoparticle has a core having the formula MX, where:
M is one or more materials selected from the group consisting of cadmium, zinc, magnesium, mercury, aluminum, gallium, indium, and thallium; and
X is one or more materials selected from the group consisting of oxygen, sulfur, selenium, tellurium, nitrogen, phosphorus, arsenic, and antimony.

97. The method of claim 84, wherein said nanoparticle comprises a core and a shell, wherein said shell comprises a semiconductor overcoating said core.

98. The method of claim 84, wherein said nanoparticle comprises a CdSe core and a ZnS shell and an SiO₂ hydrophilic coating.

99. The method of claim 84, wherein said nanoparticle has a characteristic dimension of less than about 30 nm.

100. The method according to any of claims 51-70, wherein said imaging agent comprises one or more agents selected from the group consisting of a magnetic resonance (MRI) imaging agent, a positron emission (PET) imaging agent, an electron spin resonance (ESR) imaging agent, and a near infrared (NIR) imaging agent.

101. The method according to claim 84, wherein said targeting moiety comprises one or more moieties selected from the group consisting of a nucleic acid, a peptide, an enzyme, a lipid, an antibody, a polysaccharide, a lectin, a selectin, a sugar, an aptamers, a drug, and a receptor ligand.

102. The method according to claim 101, wherein said targeting moiety is an antibody that binds an antigen selected from the group consisting of, a gastrointestinal cancer cell surface antigen, a lung cancer cell surface antigen, a brain tumor cell surface antigen, a glioma cell surface antigen, a breast cancer cell surface antigen, an esophageal cancer cell surface antigen, a common epithelial cancer cell surface antigen, a common sarcoma cell surface antigen, an osteosarcoma cell surface antigen, a fibrosarcoma cell surface antigen, a melanoma cell surface antigen, a gastric cancer cell surface antigen, a pancreatic cancer cell surface antigen, a colorectal cancer cell surface antigen, a urinary bladder cancer cell surface antigen, a prostatic cancer cell surface antigen, a renal cancer cell surface antigen, an ovarian cancer cell surface antigen, a testicular cancer cell surface antigen, an endometrial cancer cell surface antigen, a cervical cancer cell surface antigen, a Hodgkin's disease cell surface antigen, a lymphoma cell surface antigen, a leukemic cell surface antigen or a trophoblastic tumor cell surface antigen.

103. The method according to claim 101, wherein said targeting moiety is an antibody that binds an antigen selected from the group consisting of 5 alpha reductase, α-fetoprotein, AM-1, APC, APRIL, BAGE, β-catenin, Bc12, bcr-abl (b3a2), CA-125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, Cox-2, DCC, DcR3, E6/E7, ErbB2, EGFR, EMBP, Ena78, FGF8b and FGF8a, FLK-1/KDR, Folic Acid Receptor, G250, GAGE-Family, gastrin 17, GD2/GD3/GM2, GnRH, GnTV, gp100/Pmel17, gp-100-in4, gp 15, gp75/TRP-1, hCG, Heparanase, Her2/neu, HER3, Her4, HMTV, HLA-DR10, Hsp70, hTERT , IGFR1, IL-13R, iNOS, Ki 67, KIAA0205, K-ras, H-ras, N-ras, KSA, (C017-1A), LDLR-FUT, MAGE Family (MAGE1, MAGE3, etc.), Mammaglobin, MAP17, Melan-A/, MART-1, mesothelin, MIC A/B, MT-MMP's, such as MMP2, MMP3, MMP7, MMP9, Mox1, MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin, PSA, PSM, RAGE-1, Rb, RCAS1, SART-1, SSX gene , family, STAT3, STn, TAG-72, TGF-α, TGF-β, and Thymosin β 15, nucleolin, Ca15-3, astro Intestinal Tumor Antigen (Ca19-9), ovarian Tumor Antigen (Ca125), Tag72-4 Antigen (CA72-4). and carcinoembryonic antigen (CEA).

104. The method of claim 84, wherein said therapeutic moiety comprises one or more moieties selected from the group consisting of a photosensitizer, a radiosensitizer, an ESR heating moiety, an isotope, a cytotoxin, and a cancer drug.

105. A multimodal probe comprised of a water soluble, silica-coated nanoparticle exhibiting an imaging agent, targeting agent and a therapeutic agent.

106. The probe of claim 105, wherein the nanoparticle comprises an inorganic core embedded into an ultra-thin silica shell, wherein the inorganic core is comprised of semiconductor material elements of Groups II-VI.

107. The multimodal probe of claim 106 wherein the inorganic core of the nanoparticle comprise a CdSe core and a ZnS shell which further comprises a SiO₂ hydrophilic coating.

108. The multimodal probes of claim 105 wherein the nanoparticle is linked to said imaging agent, targeting agent and therapeutic agent by a linking agent.

109. The multimodal probes of claim 108, wherein the linking agent is a chelated paramagnetic ion or labeled chelator, a heterobifunctional crosslinker, functional groups, affinity agents, stabilizing groups, and combinations thereof.

110. The multimodal probes of claim 105 wherein the imaging agent is an MRI, PET or deep tissue Near Infrared (NIR) imaging agent.

111. The multimodal probes of claim 110, wherein the imaging agent is an MRI imaging agent selected from the group consisting of gadolinium, xenon, iron oxide, copper, Gd³⁺-DOTA, and ⁶⁴Cu²⁺-DOTA.

112. The multimodal probes of claim 110, wherein the imaging agent is a PET imaging agent selected from the group consisting of [¹¹C]choline, [¹⁸F]fluorodeoxyglucose (FDG), [¹¹C]methionine, [¹¹C]choline, [¹¹C]acetate, [¹⁸F]fluorocholine, and other radionuclides labeled with ⁶⁴Cu or ⁶⁸Ge.

113. The multimodal probes of claim 105, wherein the targeting agent is selected from the group consisting of nucleic acids, oligonucleotides, peptides, proteins, enzymes, lipids, antibodies, polysaccharides, lectins, selectins, and small molecules such as sugars, aptamers, drugs, and ligands.

114. The multimodal probe of claim 112, wherein the targeting agent is an antibody or a signaling peptide.

115. The multimodal probes of claim 105, wherein the therapeutic agent is selected from the group consisting of: nucleic acids (both monomeric and oligomeric), peptides, proteins, enzymes, lipids, antibodies, polysaccharides, lectins, selectins, and small molecules such as sugars, aptamers, drugs, and ligands.

116. The multimodal probe of claim 114, wherein the therapeutic agent is an antibody, drug or photosensitizer.

117. A multimodal probe for in vivo imaging and therapy that, (1) detects diseased cells by MRI, PET or deep tissue Near Infrared (NIR) imaging, and is capable of detecting diseased cells with greater sensitivity than is possible with existing technologies, (2) targets molecules that localize to normal or diseased cells, and (3) initiates apoptosis of diseased cells.

118. A nanoparticle-based technology platform for multimodal cancer imaging and therapy that, (1) detects cancer by MRI, ESR, PET, or deep tissue Near Infrared (NIR) imaging, and is capable of detecting cancer cells with greater sensitivity than is possible with existing technologies, (2) targets molecules that localize on the surface of cancer cells, and (3) initiates apoptosis of cancer cells by local infrared laser-mediated photodynamic therapy (PDT).

119. A method of increasing the relaxivity of an NMR, MRI, PET, or ESR imaging agent, said method comprising coupling said agent to a a nanoparticle.

120. The method of claim 119 wherein said nanoparticle is coated with a coating comprising silica.
